SEQUENCE LISTING (207 sequences)

SEQ ID NO: 1 - protein, 217 aa, Homo sapiens
  REGION 1..200: note = CH2-CH3 Domain of Human IgG1
  VARIANT 200: note = X is Lysine (K) or is Absent
  VARIANT 217: note = X can be any naturally occurring amino acid
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 2 - protein, 216 aa, Homo sapiens
  REGION 1..216: note = CH2-CH3 Domain of Human IgG2
  VARIANT 216: note = X is Lysine (K) or is Absent
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGX

SEQ ID NO: 3 - protein, 217 aa, Homo sapiens
  REGION 1..217: note = CH2-CH3 Domain of Human IgG3
  VARIANT 217: note = X is Lysine (K) or is Absent
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGX

SEQ ID NO: 4 - protein, 217 aa, Homo sapiens
  REGION 1..217: note = CH2-CH3 Domain of Human IgG4
  VARIANT 217: note = X is Lysine (K) or is Absent
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX

SEQ ID NO: 5 - protein, 819 aa, Homo sapiens
  REGION 1..819: note = Representative Human ADAM9 Polypeptide (NCBI Sequence NP_003807)
  SIGNAL 1..28: note = Signal Sequence
MGSGARFPSGTLRVRWLLLLGLVGPVLGAARPGFQQTSHLSSYEIITPWRLTRERREAPRPYSKQVSYVIQAEGKEHIIHLERNKDLLPEDFVVYTYNKEGTLITDHPNIQNHCHYRGYVEGVHNSSIALSDCFGLRGLLHLENASYGIEPLQNSSHFEHIIYRMDDVYKEPLKCGVSNKDIEKETAKDEEEEPPSMTQLLRRRRAVLPQTRYVELFIVVDKERYDMMGRNQTAVREEMILLANYLDSMYIMLNIRIVLVGLEIWTNGNLINIVGGAGDVLGNFVQWREKFLITRRRHDSAQLVLKKGFGGTAGMAFVGTVCSRSHAGGINVFGQITVETFASIVAHELGHNLGMNHDDGRDCSCGAKSCIMNSGASGSRNFSSCSAEDFEKLTLNKGGNCLLNIPKPDEAYSAPSCGNKLVDAGEECDCGTPKECELDPCCEGSTCKLKSFAECAYGDCCKDCRFLPGGTLCRGKTSECDVPEYCNGSSQFCQPDVFIQNGYPCQNNKAYCYNGMCQYYDAQCQVIFGSKAKAAPKDCFIEVNSKGDRFGNCGFSGNEYKKCATGNALCGKLQCENVQEIPVFGIVPAIIQTPSRGTKCWGVDFQLGSDVPDPGMVNEGTKCGAGKICRNFQCVDASVLNYDCDVQKKCHGHGVCNSNKNCHCENGWAPPNCETKGYGGSVDSGPTYNEMNTALRDGLLVFFFLIVPLIVCAIFIFIKRDQLWRSYFRKKRSQTYESDGKNQANPSRQPGSVPRHVSPVTPPREVPIYANRFAVPTYAAKQPQQFPSRPPPPQPKVSSQGNLIPARPAPAPPLYSSLT

SEQ ID NO: 6 - protein, 819 aa, Macaca fascicularis
  REGION 1..819: note = Representative Cynomolgus Monkey ADAM9 Polypeptide (NCBI Sequence XM_005563126.2)
  SIGNAL 1..28: note = Possible Signal Sequence
MGSGVGSPSGTLRVRWLLLLCLVGPVLGAARPGFQQTSHLSSYEIITPWRLTRERREAPRPYSKQVSYLIQAEGKEHIIHLERNKDLLPEDFVVYTYNKEGTVITDHPNIQNHCHFRGYVEGVYNSSVALSNCFGLRGLLHLENASYGIEPLQNSSHFEHIIYRMDDVHKEPLKCGVSNKDIEKETTKDEEEEPPSMTQLLRRRRAVLPQTRYVELFIVVDKERYDMMGRNQTAVREEMILLANYLDSMYIMLNIRIVLVGLEIWTNGNLINIAGGAGDVLGNFVQWREKFLITRRRHDSAQLVLKKGFGGTAGMAFVGTVCSRSHAGGINVFGHITVETFASIVAHELGHNLGMNHDDGRDCSCGAKSCIMNSGASGSRNFSSCSAEDFEKLTLNKGGNCLLNIPKPDEAYSAPSCGNKLVDAGEECDCGTPKECELDPCCEGSTCKLKSFAECAYGDCCKDCRFLPGGTLCRGKTSECDVPEYCNGSSQFCQPDVFIQNGYPCQNNKAYCYNGMCQYYDAQCQVIFGSKAKAAPKDCFIEVNSKGDRFGNCGFSGNEYKKCATGNALCGKLQCENVQEIPVFGIVPAIIQTPSRGTKCWGVDFQLGSDVPDPGMVNEGTKCGADKICRNFQCVDASVLNYDCDIQKKCHGHGVCNSNKNCHCENGWAPPNCETKGYGGSVDSGPTYNEMNTALRDGLLVFFFLIVPLIVCAIFIFIKRDQLWRRYFRKKRSQTYESDGKNQANPSRQPVSVPRHVSPVTPPREVPIYANRFPVPTYAAKQPQQFPSRPPPPQPKVSSQGNLIPARPAPAPPLYSSLT

SEQ ID NO: 7 - protein, 123 aa, Mus musculus
  REGION 1..123: note = VH Domain of Murine Anti-ADAM9 Antibody MAB-A
QVQLQQPGAELVKPGASVKLSCKASGYTFTSYWMHWVKQRPGQGLEWIGEIIPINGHTNYNEKFKSKATLTLDKSSSTAYMQLSSLASEDSAVYYCARGGYYYYGSRDYFDYWGQGTTLTVSS

SEQ ID NO: 8 - protein, 5 aa, Mus musculus
  REGION 1..5: note = CDRH1 Domain of Anti-ADAM9 Antibody MAB-A
SYWMH

SEQ ID NO: 9 - protein, 17 aa, Mus musculus
  REGION 1..17: note = CDRH2 Domain of Anti-ADAM9 Antibody MAB-A
EIIPINGHTNYNEKFKS

SEQ ID NO: 10 - protein, 14 aa, Mus musculus
  REGION 1..14: note = CDRH3 Domain of Anti-ADAM9 Antibody MAB-A
GGYYYYGSRDYFDY

SEQ ID NO: 11 - protein, 111 aa, Mus musculus
  REGION 1..111: note = VL Domain of Murine Anti-ADAM9 Antibody MAB-A
DIVLTQSPASLAVSLGQRATISCKASQSVDYDGDSYMNWYQQIPGQPPKLLIYAASDLESGIPARFSGSGSGTDFTLNIHPVEEEDAATYYCQQSHEDPFTFGGGTKLEIK

SEQ ID NO: 12 - protein, 15 aa, Mus musculus
  REGION 1..15: note = CDRL1 Domain of Anti-ADAM9 Antibody MAB-A
KASQSVDYDGDSYMN

SEQ ID NO: 13 - protein, 7 aa, Mus musculus
  REGION 1..7: note = CDRL2 Domain of Anti-ADAM9 Antibody MAB-A
AASDLES

SEQ ID NO: 14 - protein, 9 aa, Mus musculus
  REGION 1..9: note = CDRL3 Domain of Anti-ADAM9 Antibody MAB-A
QQSHEDPFT

SEQ ID NO: 15 - protein, 123 aa, synthetic construct
  REGION 1..123: note = Humanized/Optimized VH Domain of Murine Anti-ADAM9 Antibody MAB-A
  VARIANT 34: note = X is Methionine (M) or Isoleucine (I)
  VARIANT 55: note = X is Asparagine (N) or Phenylalanine (F)
  VARIANT 63: note = X is Lysine (K) or Arginine (R)
  VARIANT 65: note = X is Lysine (K) or Glutamine (Q)
  VARIANT 66: note = X is Serine (S) or Glycine (E)
  VARIANT 105: note = X is Proline (P), Phenylalanine (F), Tyrosine (Y), Tryptophan (W), Isoleuciine (I), Leucine (L), Valine (V), Threonine (T), Glycine (G) or Aspartate (D)
  VARIANT 106: note = X is Lysine (K), Arginine (R), Glycine (G), Asparagine (N), Histidine (H), or Serine (S)
  VARIANT 107: note = X is Phenylalanine (F), Methionine (M), Serine (S), Lysine (K) or Asparagine (N)
  VARIANT 108: note = X is Glycine (G) or Alanine (A)
  VARIANT 109: note = X is Valine (V), Threonine (T), Tryptophan (W) or Phenylalanine (F)
  VARIANT 109: note = X is Methionine (M), Leucine (L) or Lysine (K)
  VARIANT 110: note = X can be any naturally occurring amino acid
EVQLVESGGGLVKPGGSLRLSCAASGFTFSSYWXHWVRQAPGKGLEWVGEIIPIXGHTNYNEXFXXRFTISLDNSKNTLYLQMGSLRAEDTAVYYCARGGYYYYXXXXXXDYWGQGTTVTVSS

SEQ ID NO: 16 - protein, 123 aa, synthetic construct
  REGION 1..123: note = VH(1) Domain of Humanized/Optimized Anti-ADAM9 Antibody hMAB-A (hMAB-A VH(1))
EVQLVESGGGLVKPGGSLRLSCAASGFTFSSYWMHWVRQAPGKGLEWVGEIIPINGHTNYNEKFKSRFTISLDNSKNTLYLQMGSLRAEDTAVYYCARGGYYYYGSRDYFDYWGQGTTVTVSS

SEQ ID NO: 17 - protein, 123 aa, synthetic construct
  REGION 1..123: note = VH(2) Domain of Humanized/Optimized Anti-ADAM9 Antibody hMAB-A (hMAB-A VH(2))
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYGSRDYFDYWGQGTTVTVSS

SEQ ID NO: 18 - protein, 123 aa, synthetic construct
  REGION 1..123: note = VH(3) Domain of Humanized/Optimized Anti-ADAM9 Antibody hMAB-A (hMAB-A VH(3))
EVQLVESGGGLVKPGGSLRLSCAASGFTFSSYWMHWVRQAPGKGLEWVGEIIPIFGHTNYNERFQGRFTISLDNSKNTLYLQMGSLRAEDTAVYYCARGGYYYYGSRDYFDYWGQGTTVTVSS

SEQ ID NO: 19 - protein, 123 aa, synthetic construct
  REGION 1..123: note = VH(4) Domain of Humanized/Optimized Anti-ADAM9 Antibody hMAB-A (hMAB-A VH(4))
EVQLVESGGGLVKPGGSLRLSCAASGFTFSSYWIHWVRQAPGKGLEWVGEIIPIFGHTNYNERFQGRFTISLDNSKNTLYLQMGSLRAEDTAVYYCARGGYYYYGSRDYFDYWGQGTTVTVSS

SEQ ID NO: 20 - protein, 123 aa, synthetic construct
  REGION 1..123: note = VH(2A) Domain of Humanized/Optimized Anti-ADAM9 Antibody hMAB-A (hMAB-A VH(2A))
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYFNSGTLDYWGQGTTVTVSS

SEQ ID NO: 21 - protein, 123 aa, synthetic construct
  REGION 1..123: note = VH(2B) Domain of Humanized/Optimized Anti-ADAM9 Antibody hMAB-A (hMAB-A VH(2B))
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYIGKGVLDYWGQGTTVTVSS

SEQ ID NO: 22 - protein, 123 aa, synthetic construct
  REGION 1..123: note = VH(2C) Domain of Humanized/Optimized Anti-ADAM9 Antibody hMAB-A (hMAB-A VH(2C))
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYPRFGWLDYWGQGTTVTVSS

SEQ ID NO: 23 - protein, 123 aa, synthetic construct
  REGION 1..123: note = VH(2D) Domain of Humanized/Optimized Anti-ADAM9 Antibody hMAB-A (hMAB-A VH(2D))
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYTGKGVLDYWGQGTTVTVSS

SEQ ID NO: 24 - protein, 123 aa, synthetic construct
  REGION 1..123: note = VH(2E) Domain of Humanized/Optimized Anti-ADAM9 Antibody hMAB-A (hMAB-A VH(2E))
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYDSNAVLDYWGQGTTVTVSS

SEQ ID NO: 25 - protein, 123 aa, synthetic construct
  REGION 1..123: note = VH(2F) Domain of Humanized/Optimized Anti-ADAM9 Antibody hMAB-A (hMAB-A VH(2F))
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYFHSGTLDYWGQGTTVTVSS

SEQ ID NO: 26 - protein, 123 aa, synthetic construct
  REGION 1..123: note = VH(2G) Domain of Humanized/Optimized Anti-ADAM9 Antibody hMAB-A (hMAB-A VH(2G))
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYFNKAVLDYWGQGTTVTVSS

SEQ ID NO: 27 - protein, 123 aa, synthetic construct
  REGION 1..123: note = VH(2H) Domain of Humanized/Optimized Anti-ADAM9 Antibody hMAB-A (hMAB-A VH(2H))
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYGGSGVLDYWGQGTTVTVSS

SEQ ID NO: 28 - protein, 123 aa, synthetic construct
  REGION 1..123: note = VH(2I) Domain of Humanized/Optimized Anti-ADAM9 Antibody hMAB-A (hMAB-A VH(2I))
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYPRQGFLDYWGQGTTVTVSS

SEQ ID NO: 29 - protein, 123 aa, synthetic construct
  REGION 1..123: note = VH(2J) Domain of Humanized/Optimized Anti-ADAM9 Antibody hMAB-A (hMAB-A VH(2J))
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYYNSGTLDYWGQGTTVTVSS

SEQ ID NO: 30 - protein, 30 aa, synthetic construct
  REGION 1..30: note = FRH1 Domain of Humanized/Optimized Murine Anti-ADAM9 Antibody MAB-A
EVQLVESGGGLVKPGGSLRLSCAASGFTFS

SEQ ID NO: 31 - protein, 14 aa, synthetic construct
  REGION 1..14: note = FRH2 Domain of Humanized/Optimized Murine Anti-ADAM9 Antibody MAB-A
WVRQAPGKGLEWVG

SEQ ID NO: 32 - protein, 32 aa, synthetic construct
  REGION 1..32: note = FRH3 Domain of Humanized/Optimized Murine Anti-ADAM9 Antibody MAB-A
RFTISLDNSKNTLYLQMGSLRAEDTAVYYCAR

SEQ ID NO: 33 - protein, 11 aa, synthetic construct
  REGION 1..11: note = FRH4 Domain of Humanized/Optimized Murine Anti-ADAM9 Antibody MAB-A
WGQGTTVTVSS

SEQ ID NO: 34 - protein, 5 aa, synthetic construct
  REGION 1..5: note = Alternative CDRH1 Domain of Anti-ADAM9-Antibody MAB-A
SYWIH

SEQ ID NO: 35 - protein, 17 aa, synthetic construct
  REGION 1..17: note = Alternative CDRH2 Domain of Anti-ADAM9-Antibody MAB-A
EIIPIFGHTNYNEKFKS

SEQ ID NO: 36 - protein, 17 aa, synthetic construct
  REGION 1..17: note = Alternative CDRH2 Domain of Anti-ADAM9-Antibody MAB-A
EIIPIFGHTNYNERFQG

SEQ ID NO: 37 - protein, 14 aa, synthetic construct
  REGION 1..14: note = Alternative CDRH3 Domain of Anti-ADAM9-Antibody MAB-A
GGYYYYFNSGTLDY

SEQ ID NO: 38 - protein, 14 aa, synthetic construct
  REGION 1..14: note = Alternative CDRH3 Domain of Anti-ADAM9-Antibody MAB-A
GGYYYYIGKGVLDY

SEQ ID NO: 39 - protein, 14 aa, synthetic construct
  REGION 1..14: note = Alternative CDRH3 Domain of Anti-ADAM9-Antibody MAB-A
GGYYYYPRFGWLDY

SEQ ID NO: 40 - protein, 14 aa, synthetic construct
  REGION 1..14: note = Alternative CDRH3 Domain of Anti-ADAM9-Antibody MAB-A
GGYYYYTGKGVLDY

SEQ ID NO: 41 - protein, 14 aa, synthetic construct
  REGION 1..14: note = Alternative CDRH3 Domain of Anti-ADAM9-Antibody MAB-A
GGYYYYDSNAVLDY

SEQ ID NO: 42 - protein, 14 aa, synthetic construct
  REGION 1..14: note = Alternative CDRH3 Domain of Anti-ADAM9-Antibody MAB-A
GGYYYYFHSGTLDY

SEQ ID NO: 43 - protein, 14 aa, synthetic construct
  REGION 1..14: note = Alternative CDRH3 Domain of Anti-ADAM9-Antibody MAB-A
GGYYYYFNKAVLDY

SEQ ID NO: 44 - protein, 14 aa, synthetic construct
  REGION 1..14: note = Alternative CDRH3 Domain of Anti-ADAM9-Antibody MAB-A
GGYYYYGGSGVLDY

SEQ ID NO: 45 - protein, 14 aa, synthetic construct
  REGION 1..14: note = Alternative CDRH3 Domain of Anti-ADAM9-Antibody MAB-A
GGYYYYPRQGFLDY

SEQ ID NO: 46 - protein, 14 aa, synthetic construct
  REGION 1..14: note = Alternative CDRH3 Domain of Anti-ADAM9-Antibody MAB-A
GGYYYYYNSGTLDY

SEQ ID NO: 47 - protein, 5 aa, synthetic construct
  REGION 1..5: note = Consensus CDRH1 Domain of Anti-ADAM9 Antibody MAB-A
  VARIANT 4: note = X is Methionine (M) or Serine (S)
SYWXH

SEQ ID NO: 48 - protein, 17 aa, synthetic construct
  REGION 1..17: note = Consensus CDRH2 Domain of Anti-ADAM9-Antibody MAB-A
  VARIANT 6: note = X is Asparagine (N) or Phenylalanine (F)
  VARIANT 14: note = X is Lysine (K) or Arginine (R)
  VARIANT 16: note = X is Lysine (K) or Gluatamine (Q)
  VARIANT 17: note = X is Serine (S) or Glycine (G)
EIIPIXGHTNYNEXFXX

SEQ ID NO: 49 - protein, 14 aa, synthetic construct
  REGION 1..14: note = Consensus CDRH3 Domain of Anti-ADAM9-Antibody MAB-A
  VARIANT 7: note = X is Proline (P), Phenylalanine (F), Tyrosine (Y), Tryptophan (W), Isoleucine (I), Leucine (L), Valine (V), Threonine (T), Glycine (G) or Asparatate (D)
  VARIANT 8: note = X is Lysine (K), Arginine (R), Glycine (G), Asparagine (N), Histidine (H), or Serine (S)
  VARIANT 9: note = X is Phenylalanine (F), Methionine (M), Serine (S), Lysine (K) or Asparagine (N)
  VARIANT 10: note = X is Glycine (G) or Alanine (A)
  VARIANT 11: note = X is Valine (V), Threonine (T), Tryptophan (W) or Phenylalanine (F)
  VARIANT 12: note = X is Methionine (M), Leucine (L) or Lysine (K)
GGYYYYXXXXXXDY

SEQ ID NO: 50 - protein, 453 aa, synthetic construct
  REGION 1..453: note = Exemplary Humanized/Optimized IgG1 Heavy Chain of a Derivative/Variant of MAB-A Containing the hMAB-A VH (2) Domain
  VARIANT 453: note = X is Lysine (K) or is Absent
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYGSRDYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 51 - protein, 453 aa, synthetic construct
  REGION 1..453: note = Exemplary Humanized/Optimized IgG1 Heavy Chain of a Derivative/Variant of MAB-A Containing the hMAB-A VH (2C) Domain
  VARIANT 453: note = X is Lysine (K) or is Absent
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYPRFGWLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 52 - protein, 453 aa, synthetic construct
  REGION 1..453: note = Exemplary Humanized/Optimized IgG1 Heavy Chain of a Derivative/Variant of MAB-A Containing the hMAB-A VH (2I) Domain
  VARIANT 453: note = X is Lysine (K) or is Absent
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYPRQGFLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 53 - protein, 111 aa, synthetic construct
  REGION 1..111: note = Humanized/Optimized VL Domain of Murine Anti-ADAM9 Antibody MAB-A
  VARIANT 24: note = X is Lysine (K) or Arginine (R)
  VARIANT 32: note = X is Aspartate (D) or Serine (S))
  VARIANT 37: note = X is Methionine (M) or Leucine (L)
  VARIANT 96: note = X is Histidine (H) or Tyrosine (Y)
  VARIANT 97: note = X is Glutamate (E) or Serine (S)
  VARIANT 98: note = X is Aspattate (D) or Threonine (T)
DIVMTQSPDSLAVSLGERATISCXASQSVDYXGDSYXNWYQQKPGQPPKLLIYAASDLESGIPARFSGSGSGTDFTLTISSLEPEDFATYYCQQSXXXPFTFGQGTKLEIK

SEQ ID NO: 54 - protein, 111 aa, synthetic construct
  REGION 1..111: note = VL(1) Domain of Humanized/Optimized Anti-ADAM9 Antibody hMAB-A (hMAB-A VL(1))
DIVMTQSPDSLAVSLGERATISCKASQSVDYDGDSYMNWYQQKPGQPPKLLIYAASDLESGIPARFSGSGSGTDFTLTISSLEPEDFATYYCQQSHEDPFTFGQGTKLEIK

SEQ ID NO: 55 - protein, 111 aa, synthetic construct
  REGION 1..111: note = VL(2) Domain of Humanized/Optimized Anti-ADAM9 Antibody hMAB-A (hMAB-A VL(2))
DIVMTQSPDSLAVSLGERATISCKASQSVDYSGDSYMNWYQQKPGQPPKLLIYAASDLESGIPARFSGSGSGTDFTLTISSLEPEDFATYYCQQSHEDPFTFGQGTKLEIK

SEQ ID NO: 56 - protein, 111 aa, synthetic construct
  REGION 1..111: note = VL(3) Domain of Humanized/Optimized Anti-ADAM9 Antibody hMAB-A (hMAB-A VL(3))
DIVMTQSPDSLAVSLGERATISCRASQSVDYSGDSYMNWYQQKPGQPPKLLIYAASDLESGIPARFSGSGSGTDFTLTISSLEPEDFATYYCQQSHEDPFTFGQGTKLEIK

SEQ ID NO: 57 - protein, 111 aa, synthetic construct
  REGION 1..111: note = VL(4) Domain of Humanized/Optimized Anti-ADAM9 Antibody hMAB-A (hMAB-A VL(4))
DIVMTQSPDSLAVSLGERATISCRASQSVDYSGDSYLNWYQQKPGQPPKLLIYAASDLESGIPARFSGSGSGTDFTLTISSLEPEDFATYYCQQSYSTPFTFGQGTKLEIK

SEQ ID NO: 58 - protein, 23 aa, synthetic construct
  REGION 1..23: note = FRL1 Domain of Humanized/Optimized Murine Anti-ADAM9 Antibody MAB-A
DIVMTQSPDSLAVSLGERATISC

SEQ ID NO: 59 - protein, 15 aa, synthetic construct
  REGION 1..15: note = FRL2 Domain of Humanized/Optimized Murine Anti-ADAM9 Antibody MAB-A
WYQQKPGQPPKLLIY

SEQ ID NO: 60 - protein, 32 aa, synthetic construct
  REGION 1..32: note = FRL3 Domain of Humanized/Optimized Murine Anti-ADAM9 Antibody MAB-A
GIPARFSGSGSGTDFTLTISSLEPEDFATYYC

SEQ ID NO: 61 - protein, 10 aa, synthetic construct
  REGION 1..10: note = FRL4 Domain of Humanized/Optimized Murine Anti-ADAM9 Antibody MAB-A
FGQGTKLEIK

SEQ ID NO: 62 - protein, 15 aa, synthetic construct
  REGION 1..15: note = Alternative CDRL1 Domain of Anti-ADAM9 Antibody MAB-A
KASQSVDYSGDSYMN

SEQ ID NO: 63 - protein, 15 aa, synthetic construct
  REGION 1..15: note = Alternative CDRL1 Domain of Anti-ADAM9 Antibody MAB-A
RASQSVDYSGDSYMN

SEQ ID NO: 64 - protein, 15 aa, synthetic construct
  REGION 1..15: note = Alternative CDRL1 Domain of Anti-ADAM9 Antibody MAB-A
RASQSVDYSGDSYLN

SEQ ID NO: 65 - protein, 9 aa, synthetic construct
  REGION 1..9: note = Alternative CDRL2 Domain of Anti-ADAM9 Antibody MAB-A
QQSYSTPFT

SEQ ID NO: 66 - protein, 15 aa, synthetic construct
  REGION 1..15: note = Consensus CDRL1 Domain of Anti-ADAM9 Antibody MAB-A
  VARIANT 1: note = X is Lysine (K) or Arginine (R)
  VARIANT 9: note = X is Aspartate (D) or Serine (S)
  VARIANT 14: note = X is Methionine (M) or Leucine (L)
XASQSVDYXGDSYXN

SEQ ID NO: 67 - protein, 9 aa, synthetic construct
  REGION 1..9: note = Consensus CDRL3 Domain of Anti-ADAM9 Antibody MAB-A
  VARIANT 4: note = X is Histidine (H) or Tyrosine (Y)
  VARIANT 5: note = X is Glutamate (E) or Serine (S)
  VARIANT 6: note = X is Aspartate (D) or Threonine (T)
QQSXXXPFT

SEQ ID NO: 68 - protein, 218 aa, synthetic construct
  REGION 1..218: note = Exemplary Humanized/Optimized IgG1 Light Chain of a Derivative/Variant of MAB-A Containing the hMAB-A VL (2) Domain
DIVMTQSPDSLAVSLGERATISCKASQSVDYSGDSYMNWYQQKPGQPPKLLIYAASDLESGIPARFSGSGSGTDFTLTISSLEPEDFATYYCQQSHEDPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 8 aa, synthetic construct
  REGION 1..8: note = Peferred Intervening Spacer Peptide (Linker 1)
GGGSGGGG

SEQ ID NO: 70 - protein, 6 aa, synthetic construct
  REGION 1..6: note = Preferred Cysteine-Containing Spacer Peptide (Linker 2)
GGCGGG

SEQ ID NO: 71 - protein, 4 aa, synthetic construct
  REGION 1..4: note = Alternative Spacer Peptide (Linker 2)
GGGS

SEQ ID NO: 72 - protein, 6 aa, synthetic construct
  REGION 1..6: note = Alternative Spacer Peptide (Linker 2)
LGGGSG

SEQ ID NO: 73 - protein, 11 aa, synthetic construct
  REGION 1..11: note = Alternative Spacer Peptide (Linker 2)
GGGSGGGSGGG

SEQ ID NO: 74 - protein, 5 aa, synthetic construct
  REGION 1..5: note = Alternative Spacer Peptide (Linker 2)
ASTKG

SEQ ID NO: 75 - protein, 6 aa, synthetic construct
  REGION 1..6: note = Alternative Spacer Peptide (Linker 2)
LEPKSS

SEQ ID NO: 76 - protein, 5 aa, synthetic construct
  REGION 1..5: note = Alternative Spacer Peptide (Linker 2)
APSSS

SEQ ID NO: 77 - protein, 7 aa, synthetic construct
  REGION 1..7: note = Heterodimer-Promoting Domain
GVEPKSC

SEQ ID NO: 78 - protein, 6 aa, synthetic construct
  REGION 1..6: note = Heterodimer-Promoting Domain
VEPKSC

SEQ ID NO: 79 - protein, 6 aa, synthetic construct
  REGION 1..6: note = Heterodimer-Promoting Domain
AEPKSC

SEQ ID NO: 80 - protein, 7 aa, synthetic construct
  REGION 1..7: note = Heterodimer-Promoting Domain
GFNRGEC

SEQ ID NO: 81 - protein, 6 aa, synthetic construct
  REGION 1..6: note = Heterodimer-Promoting Domain
FNRGEC

SEQ ID NO: 82 - protein, 28 aa, synthetic construct
  REGION 1..28: note = E-Coil Heterodimer-Promoting Domain
EVAALEKEVAALEKEVAALEKEVAALEK

SEQ ID NO: 83 - protein, 28 aa, synthetic construct
  REGION 1..28: note = K-Coil Heterodimer-Promoting Domain
KVAALKEKVAALKEKVAALKEKVAALKE

SEQ ID NO: 84 - protein, 28 aa, synthetic construct
  REGION 1..28: note = Cysteine-Containing E-Coil Heterodimer-Promoting Domain
EVAACEKEVAALEKEVAALEKEVAALEK

SEQ ID NO: 85 - protein, 28 aa, synthetic construct
  REGION 1..28: note = Cysteine-Containing K-Coil Heterodimer-Promoting Domain
KVAACKEKVAALKEKVAALKEKVAALKE

SEQ ID NO: 86 - protein, 46 aa, Streptococcus agalactiae
  REGION 1..46: note = Albumin-Binding Domain 3 (ABD3) of Protein G of Streptococcus strain G148
LAEAKVLANRELDKYGVSDYYKNLIDNAKSAEGVKALIDEILAALP

SEQ ID NO: 87 - protein, 46 aa, synthetic construct; identical to SEQ ID NO: 86
  REGION 1..46: note = Deimmunized Albumin-Binding Domain 3 (ABD3) of Protein G of Streptococcus strain G148

SEQ ID NO: 88 - protein, 46 aa, synthetic construct
  REGION 1..46: note = Deimmunized Albumin-Binding Domain 3 (ABD3) of Protein G of Streptococcus strain G148
LAEAKVLANRELDKYGVSDYYKNAANNAKTVEGVKALIAEILAALP

SEQ ID NO: 89 - protein, 46 aa, synthetic construct
  REGION 1..46: note = Deimmunized Albumin-Binding Domain 3 (ABD3) of Protein G of Streptococcus strain G148
LAEAKVLANRELDKYGVSDYYKNLISNAKSVEGVKALIAEILAALP

SEQ ID NO: 90 - protein, 8 aa, synthetic construct
  REGION 1..8: note = Linker
APSSSPME

SEQ ID NO: 91 - protein, 16 aa, synthetic construct
  REGION 1..16: note = Linker
VEPKSADKTHTCPPCP

SEQ ID NO: 92 - protein, 15 aa, synthetic construct
  REGION 1..15: note = Linker
LEPKSADKTHTCPPC

SEQ ID NO: 93 - protein, 10 aa, synthetic construct
  REGION 1..10: note = Linker
DKTHTCPPCP

SEQ ID NO: 94 - protein, 13 aa, synthetic construct
  REGION 1..13: note = Linker
GGGDKTHTCPPCP

SEQ ID NO: 95 - protein, 16 aa, synthetic construct
  REGION 1..16: note = Linker
LEPKSSDKTHTCPPCP

SEQ ID NO: 96 - protein, 15 aa, Homo sapiens
  REGION 1..15: note = IgG1 Hinge Domain
EPKSCDKTHTCPPCP

SEQ ID NO: 97 - protein, 12 aa, Homo sapiens
  REGION 1..12: note = IgG2 Hinge Domain
ERKCCVECPPCP

SEQ ID NO: 98 - protein, 12 aa, Homo sapiens
  REGION 1..12: note = IgG4 Hinge Domain
ESKYGPPCPSCP

SEQ ID NO: 99 - protein, 12 aa, synthetic construct
  REGION 1..12: note = Variant IgG4 Hinge Domain
ESKYGPPCPPCP

SEQ ID NO: 100 - protein, 15 aa, synthetic construct
  REGION 1..15: note = Intervening Spacer Peptide
GGGGSGGGGSGGGGS

SEQ ID NO: 101 - protein, 107 aa, Homo sapiens [sequence omitted]

SEQ ID NO: 102 - protein, 104 aa, Homo sapiens
  REGION 1..104: note = Human IgG CL Lambda Domain
QPKAAPSVTLFPPSSEELQANKATLVCLISDFYPGAVTVAWKADSSPVKAGVETTPSKQSNNKYAASSYLSLTPEQWKSHRSYSCQVTHEGSTVEKTVAPTECS

SEQ ID NO: 103 - protein, 98 aa, Homo sapiens
  REGION 1..98: note = Human IgG1 CH1 Domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 104 - protein, 98 aa, Homo sapiens
  REGION 1..98: note = Human IgG2 CH1 Domain
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 105 - protein, 98 aa, Homo sapiens
  REGION 1..98: note = Human IgG4 CH1 Domain
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 106 - protein, 217 aa, synthetic construct
  REGION 1..217: note = Human IgG1 CH2-CH3 Domain Containing L234A and L235A Substitutions
  VARIANT 217: note = X is Lysine (K) or is Absent
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 107 - protein, 217 aa, synthetic construct
  REGION 1..217: note = IgG1 CH2-CH3 Domain Containing S442C Substitution
  VARIANT 217: note = X is Lysine (K) or is Absent
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLCLSPGX

SEQ ID NO: 108 - protein, 217 aa, synthetic construct
  REGION 1..217: note = IgG1 CH2-CH3 Domain Containing L234A, L235A and S442C Substitutions
  VARIANT 217: note = X is Lysine (K) or is Absent
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLCLSPGX

SEQ ID NO: 109 - protein, 217 aa, synthetic construct
  REGION 1..217: note = "Knob-Bearing" Variant Human IgG1 CH2-CH3 Domain Containing L234A and L235A Substitutions
  VARIANT 217: note = X is Lysine (K) or is Absent
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGX

SEQ ID NO: 110 - protein, 217 aa, synthetic construct
  REGION 1..217: note = "Hole-Bearing" Variant Human IgG1 CH2-CH3 Domain Containing L234A and L235A Substitutions
  VARIANT 217: note = X is Lysine (K) or is Absent
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGX

SEQ ID NO: 111 - protein, 118 aa, synthetic construct
  REGION 1..118: note = VH Domain of Lo-CD2a Anti-Human CD2 Antibody (ATCC Accession No: 11423)
EVQLQQSGPELQRPGASVKLSCKASGYIFTEYYMYWVKQRPKQGLELVGRIDPEDGSIDYVEKFKKKATLTADTSSNTAYMQLSSLTSEDTATYFCARGKFNYRFAYWGQGTLVTVSS

SEQ ID NO: 112 - protein, 112 aa, synthetic construct
  REGION 1..112: note = VL Domain of Lo-CD2a Anti-Human CD2 Antibody (ATCC Accession No: 11423)
DVVLTQTPPTLLATIGQSVSISCRSSQSLLHSSGNTYLNWLLQRTGQSPQPLIYLVSKLESGVPNRFSGSGSGTDFTLKISGVEAEDLGVYYCMQFTHYPYTFGAGTKLELK

SEQ ID NO: 113 - protein, 125 aa, synthetic construct
  REGION 1..125: note = VH(1) Domain of Anti-Human CD3 Antibody CD3 mAb-1 (CD3 mAb-1VH(1))
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSS

SEQ ID NO: 114 - protein, 125 aa, synthetic construct
  REGION 1..125: note = VH(2) Domain of Anti-Human CD3 Antibody CD3 mAb-1 (CD3 mAb-1VH(2))
EVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSS

SEQ ID NO: 115 - protein, 110 aa, synthetic construct
  REGION 1..110: note = VL Domain of Anti-Human CD3 Antibody CD3 mAb-1
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLG

SEQ ID NO: 116 - protein, 125 aa, synthetic construct
  REGION 1..125: note = VH Domain of Anti-Human CD3 Antibody CD3 mAb-1(D65G)
EVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSS

SEQ ID NO: 117 - protein, 125 aa, synthetic construct
  REGION 1..125: note = VH Domain of Anti-Human CD3 mAb-1 Low
EVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVTWFAYWGQGTLVTVSS

SEQ ID NO: 118 - protein, 125 aa, synthetic construct
  REGION 1..125: note = VH Domain of Anti-Human CD3 mAb-1 Fast
EVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHKNFGNSYVTWFAYWGQGTLVTVSS

SEQ ID NO: 119 - protein, 119 aa, synthetic construct [sequence omitted]

SEQ ID NO: 120 - protein, 107 aa, synthetic construct
  REGION 1..107: note = VL Domain of Anti-Human CD3 mAb-1 OKT3
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 121 - protein, 120 aa, synthetic construct
  REGION 1..120: note = VH Domain of Anti-Human CD8 mAb-1 OKT8
QVQLLESGPELLKPGASVKMSCKASGYTFTDYNMHWVKQSHGKSLEWIGYIYPYTGGTGYNQKFKNKATLTVDSSSSTAYMELRSLTSEDSAVYYCARNFRYTYWYFDVWGQGTTVTVSS

SEQ ID NO: 122 - protein, 112 aa, synthetic construct
  REGION 1..112: note = VL Domain of Anti-Human CD8 mAb-1 OKT8
DIVMTQSPASLAVSLGQRATISCRASESVDSYDNSLMHWYQQKPGQPPKVLIYLASNLESGVPARFSGSGSRTDFTLTIDPVEADDAATYYCQQNNEDPYTFGGGTKLEIKR

SEQ ID NO: 123 - protein, 121 aa, synthetic construct
  REGION 1..121: note = VH Domain of Anti-Human CD8 mAb-1 TRX2
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSS

SEQ ID NO: 124 - protein, 106 aa, synthetic construct
  REGION 1..106: note = VL Domain of Anti-Human CD8 mAb-1 TRX2
DIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIK

SEQ ID NO: 125 - protein, 118 aa, synthetic construct
  REGION 1..118: note = VH Domain of Anti-Human CD16 Antibody 3G8
QVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSA

SEQ ID NO: 126 - protein, 111 aa, synthetic construct
  REGION 1..111: note = VL Domain of Anti-Human CD16 Antibody 3G8
DTVLTQSPASLAVSLGQRATISCKASQSVDFDGDSFMNWYQQKPGQPPKLLIYTTSNLESGIPARFSASGSGTDFTLNIHPVEEEDTATYYCQQSNEDPYTFGGGTKLEIK

SEQ ID NO: 127 - protein, 117 aa, synthetic construct
  REGION 1..117: note = VH Domain of Anti-Human CD16 Antibody A9
QVQLQQSGAELVRPGTSVKISCKASGYTFTNYWLGWVKQRPGHGLEWIGDIYPGGGYTNYNEKFKGKATVTADTSSRTAYVQVRSLTSEDSAVYFCARSASWYFDVWGARTTVTVSS

SEQ ID NO: 128 - protein, 111 aa, synthetic construct
  REGION 1..111: note = VL Domain of Anti-Human CD16 Antibody A9
DIQAVVTQESALTTSPGETVTLTCRSNTGTVTTSNYANWVQEKPDHLFTGLIGHTNNRAPGVPARFSGSLIGDKAALTITGAQTEDEAIYFCALWYNNHWVFGGGTKLTVL

SEQ ID NO: 129 - protein, 120 aa, synthetic construct
  REGION 1..120: note = VH Domain of Anti-Human TCR Antibody BMA 031
QVQLVQSGAEVKKPGASVKVSCKASGYKFTSYVMHWVRQAPGQGLEWIGYINPYNDVTKYNEKFKGRVTITADKSTSTAYLQMNSLRSEDTAVHYCARGSYYDYDGFVYWGQGTLVTVSS

SEQ ID NO: 130 - protein, 106 aa, synthetic construct
  REGION 1..106: note = VL Domain of Anti-Human TCR Antibody BMA 031
EIVLTQSPATLSLSPGERATLSCSATSSVSYMHWYQQKPGKAPKRWIYDTSKLASGVPSRFSGSGSGTEFTLTISSLQPEDFATYYCQQWSSNPLTFGQGTKLEIK

SEQ ID NO: 131 - protein, 118 aa, synthetic construct
  REGION 1..118: note = VH Domain of Anti-Human NKG2D Antibody KYK-1.0
EVQLVESGGGVVQPGGSLRLSCAASGFTFSSYGMHWVRQAPGKGLEWVAFIRYDGSNKYYADSVKGRFTISRDNSKNTKYLQMNSLRAEDTAVYYCAKDRFGYYLDYWGQGTLVTVSS

SEQ ID NO: 132 - protein, 108 aa, synthetic construct
  REGION 1..108: note = VL Domain of Anti-Human NKG2D Antibody KYK-1.0
QPVLTQPSSVSVAPGETARIPCGGDDIETKSVHWYQQKPGQAPVLVIYDDDDRPSGIPERFFGSNSGNTATLSISRVEAGDEADYYCQVWDDNNDEWVFGGGTQLTVL

SEQ ID NO: 133 - protein, 121 aa, synthetic construct
  REGION 1..121: note = VH Domain of Anti-Human NKG2D Antibody KYK-2.0
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 134 - protein, 110 aa, synthetic construct
  REGION 1..110: note = VL Domain of Anti-Human NKG2D Antibody KYK-2.0
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 135 - protein, 278 aa, synthetic construct
  REGION 1..278: note = First Polypeptide Chain of ADAM9 x CD3 Bispecific Two-Chain Diabody ("DART-1")
DIVMTQSPDSLAVSLGERATISCKASQSVDYSGDSYMNWYQQKPGQPPKLLIYAASDLESGIPARFSGSGSGTDFTLTISSLEPEDFATYYCQQSHEDPFTFGQGTKLEIKGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSGGCGGGEVAALEKEVAALEKEVAALEKEVAALEK

SEQ ID NO: 136 - protein, 275 aa, synthetic construct
  REGION 1..275: note = Second Polypeptide Chain of ADAM9 x CD3 Bispecific Two-Chain Diabody ("DART-1")
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGEVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYGSRDYFDYWGQGTTVTVSSGGCGGGKVAALKEKVAALKEKVAALKEKVAALKE

SEQ ID NO: 137 - protein, 507 aa, synthetic construct
  REGION 1..507: note = First Polypeptide Chain of ADAM9 x CD3 Bispecific Three-Chain Diabody ("DART-2")
  VARIANT 507: note = X is Lysine (K) or is Absent
DIVMTQSPDSLAVSLGERATISCKASQSVDYSGDSYMNWYQQKPGQPPKLLIYAASDLESGIPARFSGSGSGTDFTLTISSLEPEDFATYYCQQSHEDPFTFGQGTKLEIKGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSASTKGEVAACEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGX

SEQ ID NO: 138 - protein, 274 aa, synthetic construct
  REGION 1..274: note = Second Polypeptide Chain of ADAM9 x CD3 Bispecific Three-Chain Diabody ("DART-2")
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGEVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYGSRDYFDYWGQGTTVTVSSASTKGKVAACKEKVAALKEKVAALKEKVAALKE

SEQ ID NO: 139 - protein, 227 aa, synthetic construct
  REGION 1..227: note = Third Polypeptide Chain of ADAM9 x CD3 Bispecific Three-Chain Diabody ("DART-2")
  VARIANT 227: note = X is Lysine (K) or is Absent
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGX

SEQ ID NO: 140 - protein, 6 aa, synthetic construct
  REGION 1..6: note = Sub-Domain Portion of CDRH3 Domain of anti-Human ADAM9 Antibody MAB-A (2.2)
GSRDYF

SEQ ID NO: 141 - protein, 6 aa, synthetic construct
  REGION 1..6: note = Variant of Sub-Domain Portion of CDRH3 Domain of Anti-Human ADAM9 Antibody hMAB-A (2.2)
DGEGVM

SEQ ID NO: 142 - protein, 6 aa, synthetic construct
  REGION 1..6: note = Variant of Sub-Domain Portion of CDRH3 Domain of Anti-Human ADAM9 Antibody hMAB-A (2.2)
FHSGLL

SEQ ID NO: 143 - protein, 6 aa, synthetic construct
  REGION 1..6: note = Variant of Sub-Domain Portion of CDRH3 Domain of Anti-Human ADAM9 Antibody hMAB-A (2.2)
FNSATL

SEQ ID NO: 144 - protein, 6 aa, synthetic construct
  REGION 1..6: note = Variant of Sub-Domain Portion of CDRH3 Domain of Anti-Human ADAM9 Antibody hMAB-A (2.2)
FNSGTL

SEQ ID NO: 145 - protein, 6 aa, synthetic construct
  REGION 1..6: note = Variant of Sub-Domain Portion of CDRH3 Domain of Anti-Human ADAM9 Antibody hMAB-A (2.2)
FNSSTL

SEQ ID NO: 146 - protein, 6 aa, synthetic construct
  REGION 1..6: note = Variant of Sub-Domain Portion of CDRH3 Domain of Anti-Human ADAM9 Antibody hMAB-A (2.2)
GKSKWL

SEQ ID NO: 147 - protein, 6 aa, synthetic construct
  REGION 1..6: note = Variant of Sub-Domain Portion of CDRH3 Domain of Anti-Human ADAM9 Antibody hMAB-A (2.2)
GMGGTL

SEQ ID NO: 148 - protein, 6 aa, synthetic construct
  REGION 1..6: note = Variant of Sub-Domain Portion of CDRH3 Domain of Anti-Human ADAM9 Antibody hMAB-A (2.2)
HAKGGM

SEQ ID NO: 149 - protein, 6 aa, synthetic construct
  REGION 1..6: note = Variant of Sub-Domain Portion of CDRH3 Domain of Anti-Human ADAM9 Antibody hMAB-A (2.2)
IGEAVL

SEQ ID NO: 150 - protein, 6 aa, synthetic construct
  REGION 1..6: note = Variant of Sub-Domain Portion of CDRH3 Domain of Anti-Human ADAM9 Antibody hMAB-A (2.2)
IGKGVF

SEQ ID NO: 151 - protein, 6 aa, synthetic construct
  REGION 1..6: note = Variant of Sub-Domain Portion of CDRH3 Domain of Anti-Human ADAM9 Antibody hMAB-A (2.2)
IGKGVL

SEQ ID NO: 152 - protein, 6 aa, synthetic construct
  REGION 1..6: note = Variant of Sub-Domain Portion of CDRH3 Domain of Anti-Human ADAM9 Antibody hMAB-A (2.2)
KHDSVL

SEQ ID NO: 153 - protein, 6 aa, synthetic construct
  REGION 1..6: note = Variant of Sub-Domain Portion of CDRH3 Domain of Anti-Human ADAM9 Antibody hMAB-A (2.2)
LNTAVM

SEQ ID NO: 154 - protein, 6 aa, synthetic construct
  REGION 1..6: note = Variant of Sub-Domain Portion of CDRH3 Domain of Anti-Human ADAM9 Antibody hMAB-A (2.2)
NGEGTL

SEQ ID NO: 155 - protein, 6 aa, synthetic construct
  REGION 1..6: note = Variant of Sub-Domain Portion of CDRH3 Domain of Anti-Human ADAM9 Antibody hMAB-A (2.2)
NGKNTL

SEQ ID NO: 156 - protein, 6 aa, synthetic construct
  REGION 1..6: note = Variant of Sub-Domain Portion of CDRH3 Domain of Anti-Human ADAM9 Antibody hMAB-A (2.2)
NSAGIL

SEQ ID NO: 157 - protein, 6 aa, synthetic construct
  REGION 1..6: note = Variant of Sub-Domain Portion of CDRH3 Domain of Anti-Human ADAM9 Antibody hMAB-A (2.2)
PKEGWM

SEQ ID NO: 158 - protein, 6 aa, synthetic construct
  REGION 1..6: note = Variant of Sub-Domain Portion of CDRH3 Domain of Anti-Human ADAM9 Antibody hMAB-A (2.2)
PKFGWK

SEQ ID NO: 159 - protein, 6 aa, synthetic construct
  REGION 1..6: note = Variant of Sub-Domain Portion of CDRH3 Domain of Anti-Human ADAM9 Antibody hMAB-A (2.2)
PKMGWV

SEQ ID NO: 160 - protein, 6 aa, synthetic construct
  REGION 1..6: note = Variant of Sub-Domain Portion of CDRH3 Domain of Anti-Human ADAM9 Antibody hMAB-A (2.2)
PRLGHL

SEQ ID NO: 161 - protein, 6 aa, synthetic construct
  REGION 1..6: note = Variant of Sub-Domain Portion of CDRH3 Domain of Anti-Human ADAM9 Antibody hMAB-A (2.2)
PSFGWA

SEQ ID NO: 162 - protein, 6 aa, synthetic construct
  REGION 1..6: note = Variant of Sub-Domain Portion of CDRH3 Domain of Anti-Human ADAM9 Antibody hMAB-A (2.2)
QAKGTM

SEQ ID NO: 163 - protein, 6 aa, synthetic construct
  REGION 1..6: note = Variant of Sub-Domain Portion of CDRH3 Domain of Anti-Human ADAM9 Antibody hMAB-A (2.2)
RGMGVM

SEQ ID NO: 164 - protein, 6 aa, synthetic construct
  REGION 1..6: note = Variant of Sub-Domain Portion of CDRH3 Domain of Anti-Human ADAM9 Antibody hMAB-A (2.2)
RKEGWM

SEQ ID NO: 165 - protein, 6 aa, synthetic construct
  REGION 1..6: note = Variant of Sub-Domain Portion of CDRH3 Domain of Anti-Human ADAM9 Antibody hMAB-A (2.2)
TGKGVL

SEQ ID NO: 166 - protein, 6 aa, synthetic construct
  REGION 1..6: note = Variant of Sub-Domain Portion of CDRH3 Domain of Anti-Human ADAM9 Antibody hMAB-A (2.2)
TGMGTL

SEQ ID NO: 167 - protein, 6 aa, synthetic construct
  REGION 1..6: note = Variant of Sub-Domain Portion of CDRH3 Domain of Anti-Human ADAM9 Antibody hMAB-A (2.2)
TGNGVM

SEQ ID NO: 168 - protein, 6 aa, synthetic construct
  REGION 1..6: note = Variant of Sub-Domain Portion of CDRH3 Domain of Anti-Human ADAM9 Antibody hMAB-A (2.2)
WNAGTF

SEQ ID NO: 169 - protein, 6 aa, synthetic construct
  REGION 1..6: note = Variant of Sub-Domain Portion of CDRH3 Domain of Anti-Human ADAM9 Antibody hMAB-A (2.2)
YHHTPL

SEQ ID NO: 170 - protein, 6 aa, synthetic construct
  REGION 1..6: note = Variant of Sub-Domain Portion of CDRH3 Domain of Anti-Human ADAM9 Antibody hMAB-A (2.2)
YQSATL

SEQ ID NO: 171 - protein, 6 aa, synthetic construct
  REGION 1..6: note = Variant of Sub-Domain Portion of CDRH3 Domain of Anti-Human ADAM9 Antibody hMAB-A (2.2)
DGKAVL

SEQ ID NO: 172 - protein, 6 aa, synthetic construct
  REGION 1..6: note = Variant of Sub-Domain Portion of CDRH3 Domain of Anti-Human ADAM9 Antibody hMAB-A (2.2)
FNKAVL

SEQ ID NO: 173 - protein, 6 aa, synthetic construct
  REGION 1..6: note = Variant of Sub-Domain Portion of CDRH3 Domain of Anti-Human ADAM9 Antibody hMAB-A (2.2)
FNSGTW

SEQ ID NO: 174 - protein, 6 aa, synthetic construct
  REGION 1..6: note = Variant of Sub-Domain Portion of CDRH3 Domain of Anti-Human ADAM9 Antibody hMAB-A (2.2)
FNTGVF

SEQ ID NO: 175 - protein, 6 aa, synthetic construct
  REGION 1..6: note = Variant of Sub-Domain Portion of CDRH3 Domain of Anti-Human ADAM9 Antibody hMAB-A (2.2)
GKSRFH

SEQ ID NO: 176 - protein, 6 aa, synthetic construct
  REGION 1..6: note = Variant of Sub-Domain Portion of CDRH3 Domain of Anti-Human ADAM9 Antibody hMAB-A (2.2)
IGKNVY

SEQ ID NO: 177 - protein, 6 aa, synthetic construct
  REGION 1..6: note = Variant of Sub-Domain Portion of CDRH3 Domain of Anti-Human ADAM9 Antibody hMAB-A (2.2)
MGKGVM

SEQ ID NO: 178 - protein, 6 aa, synthetic construct
  REGION 1..6: note = Variant of Sub-Domain Portion of CDRH3 Domain of Anti-Human ADAM9 Antibody hMAB-A (2.2)
NGESVF

SEQ ID NO: 179 - protein, 6 aa, synthetic construct
  REGION 1..6: note = Variant of Sub-Domain Portion of CDRH3 Domain of Anti-Human ADAM9 Antibody hMAB-A (2.2)
PDFGWM

SEQ ID NO: 180 - protein, 6 aa, synthetic construct
  REGION 1..6: note = Variant of Sub-Domain Portion of CDRH3 Domain of Anti-Human ADAM9 Antibody hMAB-A (2.2)
PGSGVM

SEQ ID NO: 181 - protein, 6 aa, synthetic construct
  REGION 1..6: note = Variant of Sub-Domain Portion of CDRH3 Domain of Anti-Human ADAM9 Antibody hMAB-A (2.2)
PKDAWL

SEQ ID NO: 182 - protein, 6 aa, synthetic construct
  REGION 1..6: note = Variant of Sub-Domain Portion of CDRH3 Domain of anti-Human ADAM9 Antibody MAB-A
PKFGWL

SEQ ID NO: 183 - protein, 6 aa, synthetic construct
  REGION 1..6: note = Variant of Sub-Domain Portion of CDRH3 Domain of anti-Human ADAM9 Antibody MAB-A
PKIGWH

SEQ ID NO: 184 - protein, 6 aa, synthetic construct
  REGION 1..6: note = Variant of Sub-Domain Portion of CDRH3 Domain of Anti-Human ADAM9 Antibody hMAB-A (2.2)
PKMGWA

SEQ ID NO: 185 - protein, 6 aa, synthetic construct
  REGION 1..6: note = Variant of Sub-Domain Portion of CDRH3 Domain of Anti-Human ADAM9 Antibody hMAB-A (2.2)
PKMGWM

SEQ ID NO: 186 - protein, 6 aa, synthetic construct
  REGION 1..6: note = Variant of Sub-Domain Portion of CDRH3 Domain of Anti-Human ADAM9 Antibody hMAB-A (2.2)
PQMGWL

SEQ ID NO: 187 - protein, 6 aa, synthetic construct
  REGION 1..6: note = Variant of Sub-Domain Portion of CDRH3 Domain of Anti-Human ADAM9 Antibody hMAB-A (2.2)
PRFGWL

SEQ ID NO: 188 - protein, 6 aa, synthetic construct
  REGION 1..6: note = Variant of Sub-Domain Portion of CDRH3 Domain of Anti-Human ADAM9 Antibody hMAB-A (2.2)
PRMGFL

SEQ ID NO: 189 - protein, 6 aa, synthetic construct
  REGION 1..6: note = Variant of Sub-Domain Portion of CDRH3 Domain of Anti-Human ADAM9 Antibody hMAB-A (2.2)
PRMGFM

SEQ ID NO: 190 - protein, 6 aa, synthetic construct
  REGION 1..6: note = Variant of Sub-Domain Portion of CDRH3 Domain of Anti-Human ADAM9 Antibody hMAB-A (2.2)
PSFGWM

SEQ ID NO: 191 - protein, 6 aa, synthetic construct
  REGION 1..6: note = Variant of Sub-Domain Portion of CDRH3 Domain of Anti-Human ADAM9 Antibody hMAB-A (2.2)
RREGWM

SEQ ID NO: 192 - protein, 6 aa, synthetic construct
  REGION 1..6: note = Variant of Sub-Domain Portion of CDRH3 Domain of Anti-Human ADAM9 Antibody hMAB-A (2.2)
SGEGVL

SEQ ID NO: 193 - protein, 6 aa, synthetic construct
  REGION 1..6: note = Variant of Sub-Domain Portion of CDRH3 Domain of Anti-Human ADAM9 Antibody hMAB-A (2.2)
SGNGVM

SEQ ID NO: 194 - protein, 6 aa, synthetic construct
  REGION 1..6: note = Variant of Sub-Domain Portion of CDRH3 Domain of Anti-Human ADAM9 Antibody hMAB-A (2.2)
VGKAVL

SEQ ID NO: 195 - protein, 6 aa, synthetic construct
  REGION 1..6: note = Variant of Sub-Domain Portion of CDRH3 Domain of Anti-Human ADAM9 Antibody hMAB-A VH (2E)
DSNAVL

SEQ ID NO: 196 - protein, 6 aa, synthetic construct
  REGION 1..6: note = Variant of Sub-Domain Portion of CDRH3 Domain of Anti-Human ADAM9 Antibody hMAB-A VH (2F)
FHSGTL

SEQ ID NO: 197 - protein, 6 aa, synthetic construct
  REGION 1..6: note = Variant of Sub-Domain Portion of CDRH3 Domain of Anti-Human ADAM9 Antibody hMAB-A VH (2H)
GGSGVL

SEQ ID NO: 198 - protein, 6 aa, synthetic construct
  REGION 1..6: note = Variant of Sub-Domain Portion of CDRH3 Domain of Anti-Human ADAM9 Antibody hMAB-A VH (2I)
PRQGFL

SEQ ID NO: 199 - protein, 6 aa, synthetic construct
  REGION 1..6: note = Variant of Sub-Domain Portion of CDRH3 Domain of Anti-Human ADAM9 Antibody hMAB-A VH (2J)
YNSGTL

SEQ ID NO: 200 - protein, 217 aa, synthetic construct
  REGION 1..217: note = Human IgG1 CH2-CH3 Domain Containing M252Y/S254T/T256E Substitutions
  VARIANT 217: note = X is Lysine (K) or is Absent
APELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 201 - protein, 217 aa, synthetic construct
  REGION 1..217: note = Human IgG1 CH2-CH3 Domain Containing L234A/L235A/M252Y/S254T/T256E Substitutions
  VARIANT 217: note = X is Lysine (K) or is Absent
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 202 - protein, 453 aa, synthetic construct
  REGION 1..453: note = Humanized/Optimized IgG1 Heavy Chain of hMAB-A VH (2I) Containing L234A/L235A Substitutions
  VARIANT 453: note = X is Lysine (K) or is Absent
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYPRQGFLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 203 - protein, 217 aa, synthetic construct
  REGION 1..217: note = Human IgG1 CH2-CH3 Domain Containing L234A, L235A, M252Y, S254T, T256E and S442C Substitutions
  VARIANT 217: note = X is Lysine (K) or is Absent
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLCLSPGX

SEQ ID NO: 204 - protein, 217 aa, synthetic construct
  REGION 1..217: note = "Knob-Bearing" Variant Human IgG1 CH2-CH3 Domain Containing L234A, L235A, M252Y, S254Tand T256E Substitutions
  VARIANT 217: note = X is Lysine (K) or is Absent
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGX

SEQ ID NO: 205 - protein, 217 aa, synthetic construct
  REGION 1..217: note = "Hole-Bearing" Variant Human IgG1 CH2-CH3 Domain Containing L234A, L235A, M252Y, S254Tand T256E Substitutions
  VARIANT 217: note = X is Lysine (K) or is Absent
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGX

SEQ ID NO: 206 - protein, 62 aa, Homo sapiens
  REGION 1..52: note = IgG3 Hinge Domain
ELKTPLGDTTHTCPRCPEPKSCDTPPPCPRCPEPKSCDTPPPCPRCPEPKSCDTPPPCPRCP

SEQ ID NO: 207 - protein, 98 aa, Homo sapiens
  REGION 1..98: note = Human IgG3 Domain
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRV

(19) United States
(12) Patent Application Publication
(10) Pub. No.: US 2025/0115676 A1
(43) Pub. Date: Apr. 10, 2025

(54) ADAM9-Binding Molecules, and Methods of Use Thereof

(71) Applicant: MacroGenics, Inc., Rockville, MD (US)

(72) Inventors: Deryk T. Loo, Belmont, CA (US); Juniper A. Scribner, Burlingame, CA (US); Bhaswati Barat, Derwood, MD (US); Gundo Diedrich, North Potomac, MD (US); Leslie S. Johnson, Rockville, MD (US); Ezio Bonvini, Potomac, MD (US)

(73) Assignee: MacroGenics, Inc., Rockville, MD (US)

(21) Appl. No.: 18/915,947

(22) Filed: Oct. 15, 2024

Related U.S. Application Data
(63) Continuation of application No. 17/553,941, filed on Dec. 17, 2021, now abandoned.
Application No. 17/553,941 is a continuation of application No. 16/471,449, filed on Jun. 19, 2019, now Pat. No. 11,242,402.
(60) Provisional application No. 62/438,516, filed on Dec. 23, 2016.

(51) Int. Cl. C07K 16/28; A61K 39/00; A61K 39/395; C07K 16/18; C07K 16/46

(52) U.S. Cl. CPC C07K 16/2896; C07K 16/18; C07K 16/2809; C07K 16/2815; C07K 16/468; A61K 39/395; A61K 2039/505; C07K 2317/24; C07K 2317/31; C07K 2317/33; C07K 2317/35; C07K 2317/52; C07K 2317/565; C07K 2317/567; C07K 2317/626; C07K 2317/732; C07K 2317/734; C07K 2317/76; C07K 2317/92; C07K 2317/94

(57) ABSTRACT

The present invention is directed to molecules, such as monospecific antibodies and bispecific, trispecific or multispecific binding molecules, including diabodies, BITE® molecules, and antibodies that are capable of specifically binding to “Disintegrin and Metalloproteinase Domain-containing Protein 9” (“ADAM9”). The invention particularly concerns such binding molecules that are capable of exhibiting high affinity binding to human and non-human ADAM9. The invention further particularly relates to such molecules that are thereby cross-reactive with human ADAM9 and the ADAM9 of a non-human primate (e.g., a cynomolgus monkey). The invention additionally pertains to all such ADAM9-binding molecules that comprise a Light Chain Variable (VL) Domain and/or a Heavy Chain Variable (VH) Domain that has been humanized and/or deimmunized so as to exhibit reduced immunogenicity upon administration of such ADAM9-binding molecule to a recipient subject. The invention is also directed to pharmaceutical compositions that contain any of such ADAM9-binding molecules, and to methods involving the use of any of such ADAM9-binding molecules in the treatment of cancer and other diseases and conditions.

### CROSS-REFERENCE TO RELATED APPLICATIONS

This application is a continuation of U.S. patent application Ser. No. 17/553,941 (filed Dec. 17, 2021), which is a continuation of U.S. patent application Ser. No. 16/471,449 (filed Jun. 19, 2019), which application is a § 371 National Stage Application of PCT/US2017/067770 (filed Dec. 21, 2017), which application claims priority to U.S. Patent Application Ser. No. 62/438,516 (filed Dec. 23, 2016), each of which applications is herein incorporated by reference in its entirety.

### REFERENCE TO SEQUENCE LISTING

This application contains a Sequence Listing which has been submitted electronically in .XML format and is hereby incorporated by reference in its entirety. Said .XML copy, created on Oct. 13, 2024, is named “0260-0029US3.xml” and is 304,792 bytes in size. The sequence listing contained in this .XML file is part of the specification and is hereby incorporated by reference herein in its entirety.

### FIELD OF THE INVENTION

The present invention is directed to molecules, such as monospecific antibodies and bispecific, trispecific or multispecific binding molecules, including diabodies, BITE® molecules, and antibodies that are capable of specifically binding to “Disintegrin and Metalloproteinase Domain-containing Protein 9” (“ADAM9”). The invention particularly concerns such binding molecules that are capable of exhibiting high affinity binding to human and non-human ADAM9. The invention further particularly relates to such molecules that are thereby cross-reactive with human ADAM9 and the ADAM9 of a non-human primate (e.g., a cynomolgus monkey). The invention additionally pertains to all such ADAM9-binding molecules that comprise a Light Chain Variable (VL) Domain and/or a Heavy Chain Variable (VH) Domain that has been humanized and/or deimmunized so as to exhibit reduced immunogenicity upon administration of such ADAM9-binding molecule to a recipient subject. The invention is also directed to pharmaceutical compositions that contain any of such ADAM9-binding molecules, and to methods involving the use of any of such ADAM9-binding molecules in the treatment of cancer and other diseases and conditions.

### BACKGROUND OF THE INVENTION

ADAM is a family of proteins involved in various physiologic and pathologic processes (Amendola, R. S. et al. (2015) “ADAM9 Disintegrin Domain Activates Human Neutrophils Through An Autocrine Circuit Involving Integrins And CXCR2,” J. Leukocyte Biol. 97(5):951-962; Edwars, D. R. et al. (2008) “The ADAM Metalloproteases,” Molec. Aspects Med. 29:258-289). At least 40 gene members of the family have been identified, and at least 21 of such members are believed to be functional in humans (Li, J. et al. (2016) “Overexpression of ADAM9 Promotes Colon Cancer Cells Invasion,” J. Invest. Surg. 26(3):127-133; Duffy, M. J. et al. (2011) “The ADAMs Family Of Proteases: New Biomarkers And Therapeutic Targets For Cancer?,” Clin. Proteomics 8:9:1-13; see also US Patent Publication No. 2013/0045244).

ADAM family members have a well-conserved structure with 8 domains, among which are a metalloprotease domain and an integrin-binding (disintegrin) domain (Duffy, M. J. et al. (2009) “The Role Of ADAMs In Disease Pathophysiology,” Clin. Chim. Acta 403:31-36). The ADAM metalloprotease domain acts as a sheddase and has been reported to modulate a series of biologic processes by cleaving transmembrane proteins, which then can act as soluble ligands and regulate cellular signaling (Amendola, R. S. et al. (2015) “ADAM9 Disintegrin Domain Activates Human Neutrophils Through An Autocrine Circuit Involving Integrins And CXCR2,” J. Leukocyte Biol. 97(5):951-962; Ito, N. et al. (2004) “ADAMs, A Disintegrin And Metalloproteinases, Mediate Shedding Of Oxytocinase,” Biochem. Biophys. Res. Commun. 314 (2004) 1008-1013).

ADAM9 is a member of the ADAM family of molecule. It is synthesized as an inactive form which is proteolytically cleaved to generate an active enzyme. Processing at the upstream site is particularly important for activation of the proenzyme. ADAM9 is expressed in fibroblasts (Zigrino, P. et al. (2011) “The Disintegrin-Like And Cysteine-Rich Domains Of ADAM-9 Mediate Interactions Between Melanoma Cells And Fibroblasts,” J. Biol. Chem. 286:6801-6807), activated vascular smooth muscle cells (Sun, C. et al. (2010) “ADAM15 Regulates Endothelial Permeability And Neutrophil Migration Via Src/ERK1/2 Signalling,” Cardiovasc. Res. 87:348-355), monocytes (Namba, K. et al. (2001) “Involvement Of ADAM9 In Multinucleated Giant Cell Formation Of Blood Monocytes,” Cell. Immunol. 213:104-113), activated macrophages (Oksala, N. et al. (2009) “ADAM-9, ADAM-15, And ADAM-17 Are Upregulated In Macrophages In Advanced Human Atherosclerotic Plaques In Aorta And Carotid And Femoral Arteries—Tampere Vascular Study,” Ann. Med. 41:279-290).

ADAM9's metalloprotease activity participates in the degradation of matrix components, to thereby allow migration of tumor cells (Amendola, R. S. et al. (2015) “ADAM9 Disintegrin Domain Activates Human Neutrophils Through An Autocrine Circuit Involving Integrins And CXCR2,” J. Leukocyte Biol. 97(5):951-962). Its disintegrin domain, which is highly homologous to many snake-venom disintegrins, allows the interaction between ADAM9 and integrins, and enables ADAM9 to modulate, positively or negatively, cell adhesion events (Zigrino, P. et al. (2011) “The Disintegrin-Like And Cysteine-Rich Domains Of ADAM-9 Mediate Interactions Between Melanoma Cells And Fibroblasts,” J. Biol. Chem. 286:6801-6807; Karadag, A. et al. (2006) “ADAM-9 (MDC-9/Meltringamma), A Member Of The A Disintegrin And Metalloproteinase Family, Regulates Myeloma-Cell-Induced Interleukin-6 Production In Osteoblasts By Direct Interaction With The Alpha(v)Beta5 Integrin,” Blood 107:3271-3278; Cominetti, M. R. et al. (2009) “Inhibition Of Platelets And Tumor Cell Adhesion By The Disintegrin Domain Of Human ADAM9 To Collagen I Under Dynamic Flow Conditions,” Biochimie 91:1045-1052). The ADAM9 disintegrin domain has been shown to interact with the α6β1, α6β4, αvβ5 and α9β1 integrins.

The expression of ADAM9 has been found to be relevant to disease, especially cancer. ADAM9 has been found to cleave and release a number of molecules with important roles in tumorigenesis and angiogenesis, such as TEK, KDR, EPHB4, CD40, VCAM1 and CDH5. ADAM9 is expressed by many types of tumor cells, including tumor cells of breast cancers, colon cancers, gastric cancers, gliomas, liver cancers, non-small cell lung cancers, melanomas, myelomas, pancreatic cancers and prostate cancers (Yoshimasu, T. et al. (2004) “Overexpression Of ADAM9 In Non-Small Cell Lung Cancer Correlates With Brain Metastasis,” Cancer Res. 64:4190-4196; Peduto, L. et al. (2005) “Critical Function For ADAM9 In Mouse Prostate Cancer,” Cancer Res. 65:9312-9319; Zigrino, P. et al. (2005) “ADAM-9 Expression And Regulation In Human Skin Melanoma And Melanoma Cell Lines,” Int. J. Cancer 116:853-859; Fritzsche, F. R. et al. (2008) “ADAM9 Is Highly Expressed In Renal Cell Cancer And Is Associated With Tumour Progression,” BMC Cancer 8:179:1-9; Fry, J. L. et al. (2010) “Secreted And Membrane-Bound Isoforms Of Protease ADAM9 Have Opposing Effects On Breast Cancer Cell Migration,” Cancer Res. 70, 8187-8198; Chang, L. et al. (2016) “Combined Rnai Targeting Human Stat3 And ADAM9 As Gene Therapy For Non-Small Cell Lung Cancer,” Oncology Letters 11:1242-1250; Fan, X. et al. (2016) “ADAM9 Expression Is Associate with Glioma Tumor Grade and Histological Type, and Acts as a Prognostic Factor in Lower-Grade Gliomas,” Int. J. Mol. Sci. 17:1276:1-11).

Significantly, increased ADAM9 expression has been found to correlate positively with tumor malignancy and metastatic potential (Amendola, R. S. et al. (2015) “ADAM9 Disintegrin Domain Activates Human Neutrophils Through An Autocrine Circuit Involving Integrins And CXCR2,” J. Leukocyte Biol. 97(5):951-962; Fan, X. et al. (2016) “ADAM9 Expression Is Associate with Glioma Tumor Grade and Histological Type, and Acts as a Prognostic Factor in Lower-Grade Gliomas,” Int. J. Mol. Sci. 17:1276:1-11; Li, J. et al. (2016) “Overexpression of ADAM9 Promotes Colon Cancer Cells Invasion,” J. Invest. Surg. 26(3):127-133). Additionally, ADAM9 and its secreted soluble isoform seem to be crucial for cancer cells to disseminate (Amendola, R. S. et al. (2015) “ADAM9 Disintegrin Domain Activates Human Neutrophils Through An Autocrine Circuit Involving Integrins And CXCR2,” J. Leukocyte Biol. 97(5):951-962; Fry, J. L. et al. (2010) “Secreted And Membrane-Bound Isoforms Of Protease ADAM9 Have Opposing Effects On Breast Cancer Cell Migration,” Cancer Res. 70, 8187-8198; Mazzocca, A. (2005) “A Secreted Form Of ADAM9 Promotes Carcinoma Invasion Through Tumor-Stromal Interactions,” Cancer Res. 65:4728-4738; see also U.S. Pat. Nos. 9,150,656; 7,585,634; 7,829,277; 8,101,361; and 8,445,198 and US Patent Publication No. 2009/0023149).

A number of studies have thus identified ADAM9 as a potential target for anticancer therapy (Peduto, L. (2009) “ADAM9 As A Potential Target Molecule In Cancer,” Curr. Pharm. Des. 15:2282-2287; Duffy, M. J. et al. (2009) “Role Of ADAMs In Cancer Formation And Progression,” Clin. Cancer Res. 15:1140-1144; Duffy, M. J. et al. (2011) “The ADAMs Family Of Proteases: New Biomarkers And Therapeutic Targets For Cancer?” Clin. Proteomics 8:9:1-13; Josson, S. et al. (2011) “Inhibition of ADAM9 Expression Induces Epithelial Phenotypic Alterations and Sensitizes Human Prostate Cancer Cells to Radiation and Chemotherapy,” Prostate 71(3):232-240; see also US Patent Publication Nos. 2016/0138113, 2016/0068909, 2016/0024582, 2015/0368352, 2015/0337356, 2015/0337048, 2015/0010575, 2014/0342946, 2012/0077694, 2011/0151536, 2011/0129450, 2010/0291063, 2010/0233079, 2010/0112713, 2009/0285840, 2009/0203051, 2004/0092466, 2003/0091568, and 2002/0068062, and PCT Publication Nos. WO 2016/077505, WO 2014/205293, WO 2014/186364, WO 2014/124326, WO 2014/108480, WO 2013/119960, WO 2013/098797, WO 2013/049704, and WO 2011/100362). Additionally, the expression of ADAM9 has also been found to be relevant to pulmonary disease and inflammation (see, e.g., US Patent Publication Nos. 2016/0068909; 2012/0149595; 2009/0233300; 2006/0270618; and 2009/0142301). Antibodies that bind to ADAM9 are commercially available from Abeam, Thermofisher, Sigma-Aldrich, and other companies.

However, despite all prior advances, a need remains for high affinity ADAM9-binding molecules that exhibit minimal binding to normal tissues and are capable of binding to human and non-human ADAM9 with similar high affinity. The present invention addresses this need and the need for improved therapeutics for cancer.

### SUMMARY OF THE INVENTION

The present invention is directed to molecules, such as monospecific antibodies and bispecific, trispecific or multispecific binding molecules, including diabodies, BITE® molecules, and antibodies that are capable of specifically binding to “Disintegrin and Metalloproteinase Domain-containing Protein 9” (“ADAM9”). The invention particularly concerns such binding molecules that are capable of exhibiting high affinity binding to human and non-human ADAM9. The invention further particularly relates to such molecules that are thereby cross-reactive with human ADAM9 and the ADAM9 of a non-human primate (e.g., a cynomolgus monkey). The invention additionally pertains to all such ADAM9-binding molecules that comprise a Light Chain Variable (VL) Domain and/or a Heavy Chain Variable (VH) Domain that have been humanized and/or deimmunized so as to exhibit reduced immunogenicity upon administration of such ADAM9-binding molecule to a recipient subject. The invention is also directed to pharmaceutical compositions that contain any of such ADAM9-binding molecules, and to methods involving the use of any of such ADAM9-binding molecules in the treatment of cancer and other diseases and conditions.

In detail, the invention provides an ADAM9-binding molecule that comprises an ADAM9-binding domain, wherein such ADAM9-binding domain comprises a Light Chain Variable (VL) Domain and a Heavy Chain Variable (VH) Domain, wherein such Heavy Chain Variable Domain comprises a CDR_{H}1 Domain, a CDR_{H}2 Domain and a CDR_{H}3 Domain, and such Light Chain Variable Domain comprises a CDR_{L}1 Domain, a CDR_{L}2 Domain, and a CDR_{L}3 Domain, wherein:
(A) such CDR_{H}1 Domain, CDR_{H}2 Domain and CDR_{H}3 Domain have the amino acid sequence of the CDR_{H}1 Domain, CDR_{H}2 Domain and CDR_{H}3 Domain of a Heavy Chain Variable (VH) Domain of an optimized variant of MAB-A; and such CDR_{L}1 Domain, CDR_{L}2 Domain, and CDR_{L}3 Domain have the amino acid sequence of the CDR_{L}1 Domain, CDR_{L}2 Domain, and CDR_{L}3 Domain of the Light Chain Variable (VL) Domain of MAB-A; or
(B) such CDR_{H}1 Domain, CDR_{H}2 Domain and CDR_{H}3 Domain have the amino acid sequence of the CDR_{H}1 Domain, CDR_{H}2 Domain and CDR_{H}3 Domain of the Heavy Chain Variable (VH) Domain of MAB-A; and such CDR_{L}1 Domain, CDR_{L}2 Domain, and CDR_{L}3 Domain have the amino acid sequence of the CDR_{L}1 Domain, CDR_{L}2 Domain, and CDR_{L}3 Domain of a Light Chain Variable (VL) Domain of an optimized variant of MAB-A; or
(C) such CDR_{H}1 Domain, CDR_{H}2 Domain and CDR_{H}3 Domain have the amino acid sequence of the CDR_{H}1 Domain, CDR_{H}2 Domain and CDR_{H}3 Domain of a Heavy Chain Variable (VH) Domain of an optimized variant of MAB-A; and such CDR_{L}1 Domain, CDR_{L}2 Domain, and CDR_{L}3 Domain have the amino acid sequence of the CDR_{L}1 Domain, CDR_{L}2 Domain, and CDR_{L}3 Domain of a Light Chain Variable (VL) Domain of an optimized variant of MAB-A.

The invention particularly concerns the embodiment of such ADAM9-binding molecules, wherein such ADAM9-binding domain possesses:
(A) (1) the CDR_{H}1 Domain, CDR_{H}2 Domain and CDR_{H}3 Domain of the Heavy Chain Variable (VH) Domain of MAB-A; and
   (2) the FR1, FR2, FR3 and FR4 of a VH Domain of a humanized variant of MAB-A; or
(B) (1) the CDR_{L}1 Domain, CDR_{L}2 Domain and CDR_{L}3 Domain of the Light Chain Variable (VL) Domain MAB-A; and
   (2) the FR1, FR2, FR3 and FR4 of a VL Domain of a humanized variant of MAB-A; or
(C) (1) the CDR_{H}1 Domain, CDR_{H}2 Domain and CDR_{H}3 Domain of a Heavy Chain Variable (VH) Domain of an optimized variant of MAB-A; and
   (2) the FR1, FR2, FR3 and FR4 of the VH Domain of a humanized variant of MAB-A; or
(D) (1) the CDR_{L}1 Domain, CDR_{L}2 Domain and CDR_{L}3 Domain of a Light Chain Variable (VL) Domain of an optimized variant of MAB-A; and
   (2) the FR1, FR2, FR3 and FR4 of the VL Domain of a humanized variant of MAB-A; or
(E) (1) the Heavy Chain Variable (VH) Domain of a humanized/optimized variant of MAB-A; and
   (2) the VL Light Chain Variable (VL) Domain of a humanized/optimized variant of MAB-A.

The invention additionally concerns the embodiment of all such ADAM9-binding molecules, wherein such Heavy Chain Variable (VH) Domain of such optimized variant of MAB-A comprises the amino acid sequence of SEQ ID NO:15:

EVQLVESGGG LVKPGGSLRL SCAASGFTFS [symbol] WVRQA
PGKGLEWVGE [symbol] [symbol] RFTI SLDNSKNTLY
LQMGSLRAED TAVYYCARGG [symbol]
DYWGQGTTVT VSS

wherein: X₁, X₂, X₃, X₄, X₅, and X₆ are independently selected,
wherein: X₁ is M or I; X₂ is N or F;
   X₃ is K or R; X₄ is K or Q;
   X₅ is S or G, and X₆ is P, F, Y, W, I, L, V, T, G or D;
wherein: X₇, X₈, X₉, X₁₀, and X₁₁ are selected such that:
   when X₆ is P; X₇ is K or R; X₈ is F or M; X₉ is G; X₁₀ is W or F; and X₁₁ is M, L or K;
   when X₆ is F, Y or W; X₇ is N or H; X₈ is S or K; X₉ is G or A; X₁₀ is T or V; and X₁₁ is M, L or K;
   when X₆ is I, L or V; X₇ is G; X₈ is K; X₉ is G or A; X₁₀ is V; and X₁₁ is M, L or K;
   when X₆ is T; X₇ is G; X₈ is K, M or N; X₉ is G; X₁₀ is V or T; and X₁₁ is L or M;
   when X₆ is G; X₇ is G; X₈ is S; X₉ is G; X₁₀ is V; and X₁₁ is L;
   when X₆ is D; X₇ is S; X₈ is N; X₉ is A; X₁₀ is V; and X₁₁ is L.

The invention additionally concerns the embodiment of all such ADAM9-binding molecules, wherein such CDR_{H}1 Domain, CDR_{H}2 Domain and CDR_{H}3 Domain of such Heavy Chain Variable (VH) Domain of such optimized variant of MAB-A respectively have the amino acid sequences of:

(1)
SEQ ID NO: 47
(SYWX₁H)

wherein: X₁ is M or I;

(2)
SEQ ID NO: 48
(EIIPIX₂GHTNYNEX₃FX₄X₅)

wherein: X₂, X₃, X₄, and X₅ are independently selected, and
wherein: X₂ is N or F; X₃ is K or R; X₄ is K or Q; and X₅ is S or G; and

(3)
SEQ ID NO: 49
(GGYYYYX₆X₇X₈X₉X₁₀X₁₁DY)

wherein: X₆, is P, F, Y, W, I, L, V, T, G or D, and X₇, X₈, X₉, X₁₀, and X₁₁ are selected such that:
(A) when X₆ is P:
   X₇ is K or R; X₈ is F or M; X₉ is G;
   X₁₀ is W or F; and X₁₁ is M, L or K;
(B) when X₆ is F, Y or W:
   X₇ is N or H; X₈ is S or K; X₉ is G or A;
   X₁₀ is T or V; and X₁₁ is M, L or K;
(C) when X₆ is I, L or V:
   X₇ is G; X₈ is K; X₉ is G or A;
   X₁₀ is V; and X₁₁ is M, L or K;
(D) when X₆ is T:
   X₇ is G; X₈ is K, M or N; X₉ is G;
   X₁₀ is V or T; and X₁₁ is L or M;
(E) when X₆ is G:
   X₇ is G; X₈ is S; X₉ is G;
   X₁₀ is V; and X₁₁ is L; and
(F) when X₆ is D:
   X₇ is S; X₈ is N; X₉ is A;
   X₁₀ is V; and X₁₁ is L.

The invention additionally concerns the embodiment of all such ADAM9-binding molecules, wherein such Heavy Chain Variable (VH) Domain of such optimized variant of MAB-A is selected from the group consisting of:

(SEQ ID NO: 16)
(1) hMAB-A VH(1);
(SEQ ID NO: 17)
(2) hMAB-A VH(2);
(SEQ ID NO: 18)
(3) hMAB-A VH(3);
(SEQ ID NO: 19)
(4) hMAB-A VH(4);
(SEQ ID NO: 20)
(5) hMAB-A VH(2A);
(SEQ ID NO: 21)
(6) hMAB-A VH(2B);
(SEQ ID NO: 22)
(7) hMAB-A VH(2C);
(SEQ ID NO: 23)
(8) hMAB-A VH(2D);
(SEQ ID NO: 24)
(9) hMAB-A VH(2E);
(SEQ ID NO: 25)
(10) hMAB-A VH(2F);
(SEQ ID NO: 26)
(11) hMAB-A VH(2G);
(SEQ ID NO: 27)
(12) hMAB-A VH(2H);
(SEQ ID NO: 28)
(13) hMAB-A VH(2I);
and
(SEQ ID NO: 29)
(14) hMAB-A VH(2J).

The invention additionally concerns the embodiment of all such ADAM9-binding molecules, wherein such Light Chain Variable (VL) Domain comprises the amino acid sequence of SEQ ID NO:53:

DIVMTQSPDS LAVSLGERAT ISC [symbol] [symbol]
WY QQKPGQPPKL LIYAASDLES
GIPARFSGSG SGTDFTLTIS SLEPEDFATY
YC [symbol] TFGQGTKLEI K

wherein: X₁₂, X₁₃, X₁₄, X₁₅, X₁₆, and X₁₇, are independently selected, and
wherein: X₁₂ is K or R; X₁₃ is D or S;
   X₁₄ is M or L; X₁₅ is H or Y;
   X₁₆ is E or S; and X₁₇ is D or T.

The invention additionally concerns the embodiment of all such ADAM9-binding molecules, wherein such CDR_{L}1 Domain, CDR_{L}2 Domain and CDR_{L}3 Domain of such Light Chain Variable (VL) Domain of such optimized variant of MAB-A respectively have the amino acid sequences of:

(1)
SEQ ID NO: 66
(X₁₂ASQSVDYX₁₃GDSYX₁₄N)

wherein: X₁₂, X₁₃, X₁₄, are independently selected, and
wherein: X₁₂ is K or R; X₁₃ is D or S; and X₁₄ is M or L;

(2)
SEQ ID NO: 13
(AASDLES);
and
(3)
SEQ ID NO: 67
(QQSX₁₅X₁₆X₁₇PFT)

wherein: X₁₅, X₁₆, and X₁₇, are independently selected, and
wherein: X₁₅ is H or Y; X₁₆ is E or S; and X₁₇ is D or T.

The invention additionally concerns the embodiment of such ADAM9-binding molecules, wherein such Light Chain Variable (VL) Domain of such optimized variant of MAB-A is selected from the group consisting of:

(SEQ ID NO: 54)
(1) hMAB-A VL(1);
(SEQ ID NO: 55)
(2) hMAB-A VL(2);
(SEQ ID NO: 56)
(3) hMAB-A VL(3);
(SEQ ID NO: 57)
(4) hMAB-A VL(4);
(SEQ ID NO: 20)
(5) hMAB-A VL(2A).

The invention additionally concerns the embodiment of such ADAM9-binding molecules, wherein the ADAM9-binding domain comprises:
(A) (1) a CDR_{H}1 Domain that comprises the amino acid sequence SYWMH (SEQ ID NO:8);
   (2) a CDR_{H}2 Domain that comprises the amino acid sequence EIIPIFGHTNYNEKFKS (SEQ ID NO:35); or
   (3) a CDR_{H}3 Domain that comprises the amino acid sequence GGYYYYPRQGFLDY (SEQ ID NO:45);
or
(B) (1) a CDR_{L}1 Domain that comprises the amino acid sequence KASQSVDYSGDSYMN (SEQ ID NO:62);
   (2) a CDR_{L}2 Domain that comprises the amino acid sequence AASDLES (SEQ ID NO:13); or
   (3) a CDR_{L}3 Domain that comprises the amino acid sequence QQSHEDPFT (SEQ ID NO:14);

The invention additionally concerns the embodiment of such ADAM9-binding molecules, wherein the ADAM9-binding domain comprises the CDR_{H}1 Domain that comprises the amino acid sequence SYWMH (SEQ ID NO:8), the CDR_{H}2 Domain that comprises the amino acid sequence EIIPIFGHTNYNEKFKS (SEQ ID NO:35), and the CDR_{H}3 Domain that comprises the amino acid sequence GGYYYYPRQGFLDY (SEQ ID NO:45).

The invention additionally concerns the embodiment of such ADAM9-binding molecules, wherein the ADAM9-binding domain comprises the CDR_{L}1 Domain that comprises the amino acid sequence KASQSVDYSGDSYMN (SEQ ID NO:62), the CDR_{L}2 Domain that comprises the amino acid sequence AASDLES (SEQ ID NO:13), and the CDR_{L}3 Domain that comprises the amino acid sequence QQSHEDPFT (SEQ ID NO:14).

The invention additionally concerns the embodiment of such ADAM9-binding molecules, wherein such ADAM9-binding domain comprises:
(A) the Heavy Chain Variable (VH) Domain of hMAB-A (2I.2) (SEQ ID NO:28); or
(B) the Light Chain Variable (VL) Domain of hMAB-A (2I.2) (SEQ ID NO:55); or
(C) the Heavy Chain Variable (VH) Domain of hMAB-A (2I.2) (SEQ ID NO:28) and the Light Chain Variable (VL) Domain of hMAB-A (2I.2) (SEQ ID NO:55).

The invention additionally concerns the embodiment of such ADAM9-binding molecules, wherein such ADAM9-binding domain comprises a CDR_{H}1 domain, a CDR_{H}2 domain, and a CDR_{H}3 domain and a CDR_{L}1 domain, a CDR_{L}2 domain, and a CDR_{L}3 domain having the sequences selected from the group consisting of:
(a) SEQ ID NOs:8, 35 and 10 and SEQ ID NOs:62, 13 and 14, respectively
(b) SEQ ID NOs:8, 35 and 10 and SEQ ID NOs:63, 13 and 14, respectively;
(c) SEQ ID NOs:8, 36 and 10 and SEQ ID NOs:63, 13 and 14, respectively; and
(d) SEQ ID NOs:34, 36 and 10 and SEQ ID NO:64, 13 and 65, respectively.

The invention additionally concerns the embodiment of such ADAM9-binding molecules, wherein such ADAM9-binding domain comprises a heavy chain variable domain (VH) and a light chain variable domain (VL) having sequences that are at least 90%, at least 95%, or at least 99% identical to sequences selected from the group consisting of:
(a) SEQ ID NO:17 and SEQ ID NO:55, respectively;
(b) SEQ ID NO:17 and SEQ ID NO:56, respectively;
(c) SEQ ID NO:18 and SEQ ID NO:56, respectively; and
(d) SEQ ID NO:19 and SEQ ID NO:57, respectively.

The invention additionally concerns the embodiment of such ADAM9-binding molecules, wherein such ADAM9-binding domain comprises a heavy chain variable domain (VH) and a light chain variable domain (VL) having the sequences selected from the group consisting of:
(a) SEQ ID NO:17 and SEQ ID NO:55, respectively;
(b) SEQ ID NO:17 and SEQ ID NO:56, respectively;
(c) SEQ ID NO:18 and SEQ ID NO:56, respectively; and
(d) SEQ ID NO:19 and SEQ ID NO:57, respectively.

The invention additionally concerns the embodiment of such ADAM9-binding molecules, wherein such ADAM9-binding domain has at least a 150-fold enhancement in binding affinity to cyno ADAM9 and retains high affinity binding to human ADAM9 as compared to MAB-A.

The invention additionally concerns the embodiment of such ADAM9-binding molecules, wherein such ADAM9-binding domain comprises a CDR_{H}1 domain, a CDR_{H}2 domain, and a CDR_{H}3 domain and a CDR_{L}1 domain, a CDR_{L}2 domain, and a CDR_{L}3 domain having the sequences selected from the group consisting of:
(a) SEQ ID NOs:8, 35 and 37 and SEQ ID NOs:62, 13 and 14, respectively;
(b) SEQ ID NOs:8, 35 and 38 and SEQ ID NOs:62, 13 and 14, respectively;
(c) SEQ ID NOs:8, 35 and 39 and SEQ ID NOs:62, 13 and 14, respectively;
(d) SEQ ID NOs:8, 35 and 40 and SEQ ID NOs:62, 13 and 14, respectively;
(e) SEQ ID NOs:8, 35 and 41 and SEQ ID NOs:62, 13 and 14, respectively;
(f) SEQ ID NOs:8, 35 and 42 and SEQ ID NOs:62, 13 and 14, respectively;
(g) SEQ ID NOs:8, 35 and 43 and SEQ ID NOs:62, 13 and 14, respectively;
(h) SEQ ID NOs:8, 35 and 44 and SEQ ID NOs:62, 13 and 14, respectively;
(i) SEQ ID NOs:8, 35 and 45 and SEQ ID NOs:62, 13 and 14, respectively; and
(j) SEQ ID NOs:8, 35 and 46 and SEQ ID NOs:62, 13 and 14, respectively.

The invention additionally concerns the embodiment of such ADAM9-binding molecules, wherein such ADAM9-binding domain comprises a heavy chain variable domain (VH) and a light chain variable domain (VL) having sequences that are at least 90%, at least 95%, or at least 99% identical to sequences selected from the group consisting of:
(a) SEQ ID NO:20 and SEQ ID NO:55, respectively;
(b) SEQ ID NO:21 and SEQ ID NO:55, respectively;
(c) SEQ ID NO:22 and SEQ ID NO:55, respectively;
(d) SEQ ID NO:23 and SEQ ID NO:55, respectively;
(e) SEQ ID NO:24 and SEQ ID NO:55, respectively;
(f) SEQ ID NO:25 and SEQ ID NO:55, respectively;
(g) SEQ ID NO:26 and SEQ ID NO:55, respectively;
(h) SEQ ID NO:27 and SEQ ID NO:55, respectively;
(i) SEQ ID NO:28 and SEQ ID NO:55, respectively; and
(j) SEQ ID NO:29 and SEQ ID NO:55, respectively.

The invention additionally concerns the embodiment of such ADAM9-binding molecules, wherein such ADAM9-binding domain comprises a heavy chain variable domain (VH) and a light chain variable domain (VL) having the sequences selected from the group consisting of:
(a) SEQ ID NO:20 and SEQ ID NO:55, respectively;
(b) SEQ ID NO:21 and SEQ ID NO:55, respectively;
(c) SEQ ID NO:22 and SEQ ID NO:55, respectively;
(d) SEQ ID NO:23 and SEQ ID NO:55, respectively;
(e) SEQ ID NO:24 and SEQ ID NO:55, respectively;
(f) SEQ ID NO:25 and SEQ ID NO:55, respectively;
(g) SEQ ID NO:26 and SEQ ID NO:55, respectively;
(h) SEQ ID NO:27 and SEQ ID NO:55, respectively;
(i) SEQ ID NO:28 and SEQ ID NO:55, respectively; and
(j) SEQ ID NO:29 and SEQ ID NO:55, respectively.

The invention additionally concerns the embodiment of all such ADAM9-binding molecules, wherein such molecule is a monospecific ADAM9-binding antibody or an ADAM9-binding fragment thereof, or wherein such molecule is a bispecific antibody.

The invention additionally concerns the embodiment of all such ADAM9-binding molecules, wherein such molecule is a diabody, such diabody being a covalently bonded complex that comprises two, three, four or five polypeptide chains.

The invention additionally concerns the embodiment of all such ADAM9-binding molecules, wherein such molecule is a trivalent binding molecule, such trivalent binding molecule being a covalently bonded complex that comprises three, four, five, or more polypeptide chains.

The invention additionally concerns the embodiment of such ADAM9-binding molecules, wherein such molecule comprises an Albumin-Binding Domain (ABD).

The invention additionally concerns the embodiment of such ADAM9-binding molecules, wherein such molecule comprises an Fc Region, and particularly the embodiment wherein such Fc Region is a variant Fc Region that comprises:
(a) one or more amino acid modification(s) that reduce(s) the affinity of the variant Fc Region for an FcγR; and/or
(b) one or more amino acid modification(s) that enhance(s) the serum half-life of such ADAM9-binding molecule.

The invention additionally concerns the embodiment of such ADAM9-binding molecules, wherein such one or more amino acid modification(s) that reduce(s) the affinity of the variant Fc Region for an FcγR comprise:
(A) L234A;
(B) L235A; or
(C) L234A and L235A;
   wherein such numbering is that of the EU index as in Kabat.

The invention additionally concerns the embodiment of such ADAM9-binding molecules, wherein such one or more amino acid modification(s) that that enhance(s) the serum half-life of such ADAM9-binding molecule comprise:
(A) M252Y;
(B) M252Y and S254T;
(C) M252Y and T256E;
(D) M252Y, S254T and T256E; or
(E) K288D and H435K;
   wherein such numbering is that of the EU index as in Kabat.

The invention additionally concerns the embodiment of such ADAM9-binding molecules, wherein such molecule is bispecific and comprises an epitope-binding site capable of immunospecific binding to an epitope of ADAM9 and an epitope-binding site capable of immunospecific binding to an epitope of a molecule present on the surface of an effector cell.

The invention additionally concerns the embodiment of such ADAM9-binding molecules, wherein such molecule comprises two epitope-binding sites capable of immunospecific binding to epitope(s) of ADAM9 and two epitope-binding sites capable of immunospecific binding to epitope(s) of a molecule present on the surface of an effector cell.

The invention additionally concerns the embodiment of such ADAM9-binding molecules, wherein such molecule is trispecific and comprises:
(a) one epitope-binding site capable of immunospecific binding to an epitope of ADAM9;
(b) one epitope-binding site capable of immunospecific binding to an epitope of a first molecule present on the surface of an effector cell; and
(c) one epitope-binding site capable of immunospecific binding to an epitope of a second molecule present on the surface of an effector cell.

The invention additionally concerns the embodiment of such ADAM9-binding molecules, wherein such molecule is capable of simultaneously binding to ADAM9 and such molecule present on the surface of an effector cell.

The invention additionally concerns the embodiment of such ADAM9-binding molecules, wherein such molecule present on the surface of an effector cell is CD2, CD3, CD8, TCR, or NKG2D.

The invention additionally concerns the embodiment of such ADAM9-binding molecules, wherein such effector cell is a cytotoxic T-cell or a Natural Killer (NK) cell.

The invention additionally concerns the embodiment of such ADAM9-binding molecules, wherein such first molecule present on the surface of an effector cell is CD3 and such second molecule present on the surface of an effector cell is CD8

The invention additionally concerns the embodiment of such ADAM9-binding molecules, wherein such ADAM9-binding molecule mediates coordinated binding of a cell expressing ADAM9 and a cytotoxic T cell.

The invention additionally concerns a pharmaceutical composition that comprises an effective amount of any of the above-described ADAM9-binding molecules and a pharmaceutically acceptable carrier, excipient or diluent.

The invention additionally concerns the use of any of the above-described ADAM9-binding molecules, or the use of the above-described pharmaceutical composition in the treatment of a disease or condition associated with, or characterized by, the expression of ADAM9.

The invention particularly concerns such use wherein such disease or condition associated with, or characterized by, the expression of ADAM9 is cancer, and especially wherein such cancer is selected from the group consisting: bladder cancer, breast cancer, cervical cancer, colorectal cancer (especially an adenocarcinoma, gastrointestinal carcinoid tumors, gastrointestinal stromal tumors, primary colorectal lymphoma, leiomyosarcoma, melanoma, or squamous cell carcinoma), esophageal cancer, gastric cancer, head and neck cancer, liver cancer, non-small-cell lung cancer (especially a squamous cell carcinoma, adenocarcinoma, or large-cell undifferentiated carcinoma), myeloid cancer, ovarian cancer, pancreatic cancer, prostate cancer, renal cell carcinoma, thyroid cancer, testicular cancer, and uterine cancer.

The invention additionally concerns a method for treating a disease or condition associated with, or characterized by, the expression of ADAM9 in a subject comprising administering to such subject an effective amount of any of the above-described ADAM9-binding molecules, or any of the above-described pharmaceutical compositions.

The invention particularly concerns such method wherein such disease or condition associated with, or characterized by, the expression of ADAM9 is cancer, and especially wherein such cancer is selected from the group consisting: bladder cancer, breast cancer, cervical cancer, colorectal cancer (especially an adenocarcinoma, gastrointestinal carcinoid tumors, gastrointestinal stromal tumors, primary colorectal lymphoma, leiomyosarcoma, melanoma, or squamous cell carcinoma), esophageal cancer, gastric cancer, head and neck cancer, liver cancer, non-small-cell lung cancer (especially a squamous cell carcinoma, adenocarcinoma, or large-cell undifferentiated carcinoma), myeloid cancer, ovarian cancer, pancreatic cancer, prostate cancer, renal cell carcinoma, thyroid cancer, testicular cancer, and uterine cancer.

### BRIEF DESCRIPTION OF THE DRAWINGS

FIG. 1 provides a schematic of a representative covalently bonded diabody having two epitope-binding sites composed of two polypeptide chains, each having an E-coil or K-coil Heterodimer-Promoting Domain (alternative Heterodimer-Promoting Domains are provided below). A cysteine residue may be present in a linker and/or in the Heterodimer-Promoting Domain as shown in FIG. 3B. VL and VH Domains that recognize the same epitope are shown using the same shading or fill pattern.

FIG. 2 provides a schematic of a representative covalently bonded diabody molecule having two epitope-binding sites composed of two polypeptide chains, each having a CH2 and CH3 Domain, such that the associated chains form all or part of an Fc Region. VL and VH Domains that recognize the same epitope are shown using the same shading or fill pattern.

FIG. 3A, FIG. 3B and FIG. 3C provide schematics showing representative covalently bonded tetravalent diabodies having four epitope-binding sites composed of two pairs of polypeptide chains (i.e., four polypeptide chains in all). One polypeptide of each pair possesses a CH2 and CH3 Domain, such that the associated chains form all or part of an Fc Region. VL and VH Domains that recognize the same epitope are shown using the same shading or fill pattern. The two pairs of polypeptide chains may be same. In such embodiments wherein the two pairs of polypeptide chains are the same and the VL and VH Domains recognize different epitopes (as shown in FIG. 3A and FIG. 3B), the resulting molecule possesses four epitope-binding sites and is bispecific and bivalent with respect to each bound epitope. In such embodiments wherein the VL and VH Domains recognize the same epitope (e.g., the same VL Domain CDRs and the same VH Domain CDRs are used on both chains) the resulting molecule possesses four epitope-binding sites and is monospecific and tetravalent with respect to a single epitope. Alternatively, the two pairs of polypeptides may be different. In such embodiments wherein the two pairs of polypeptide chains are different and the VL and VH Domains of each pair of polypeptides recognize different epitopes (as shown by the different shading and patterns in FIG. 3C), the resulting molecule possesses four epitope-binding sites and is tetraspecific and monovalent with respect to each bound epitope. FIG. 3A shows an Fc Region-containing diabody which contains a peptide Heterodimer-Promoting Domain comprising a cysteine residue. FIG. 3B shows an Fc Region-containing diabody, which contains E-coil and K-coil Heterodimer-Promoting Domains comprising a cysteine residue and a linker (with an optional cysteine residue). FIG. 3C, shows an Fc-Region-Containing diabody, which contains antibody CH1 and CL domains.

FIG. 4A and FIG. 4B provide schematics of a representative covalently bonded diabody molecule having two epitope-binding sites composed of three polypeptide chains. Two of the polypeptide chains possess a CH2 and CH3 Domain, such that the associated chains form all or part of an Fc Region. The polypeptide chains comprising the VL and VH Domain further comprise a Heterodimer-Promoting Domain. VL and VH Domains that recognize the same epitope are shown using the same shading or fill pattern.

FIG. 5 provides the schematics of a representative covalently bonded diabody molecule having four epitope-binding sites composed of five polypeptide chains. Two of the polypeptide chains possess a CH2 and CH3 Domain, such that the associated chains form an Fc Region that comprises all or part of an Fc Region. The polypeptide chains comprising the linked VL and VH Domains further comprise a Heterodimer-Promoting Domain. VL and VH Domains that recognize the same epitope are shown using the same shading or fill pattern.

FIG. 6A, FIG. 6B, FIG. 6C, FIG. 6D, FIG. 6E and FIG. 6F provide schematics of representative Fc Region-containing trivalent binding molecules having three epitope-binding sites. FIG. 6A and FIG. 6B, respectively, illustrate schematically the domains of trivalent binding molecules comprising two diabody-type binding domains and a Fab-type binding domain having different domain orientations in which the diabody-type binding domains are N-terminal or C-terminal to an Fc Region. The molecules in FIG. 6A and FIG. 6B comprise four chains. FIG. 6C and FIG. 6D, respectively, illustrate schematically the domains of trivalent binding molecules comprising two diabody-type binding domains N-terminal to an Fc Region, and a Fab-type binding domain in which the light chain and heavy chain are linked via a polypeptide spacer, or an scFv-type binding domain. The trivalent binding molecules in FIG. 6E and FIG. 6F, respectively, illustrate schematically the domains of trivalent binding molecules comprising two diabody-type binding domains C-terminal to an Fc Region, and a Fab-type binding domain in which the light chain and heavy chain are linked via a polypeptide spacer, or an scFv-type binding domain. The trivalent binding molecules in FIG. 6C, FIG. 6D, FIG. 6E and FIG. 6F comprise three chains. VL and VH Domains that recognize the same epitope are shown using the same shading or fill pattern.

FIG. 7A, FIG. 7B and FIG. 7C present the results of an immunohistochemistry (IHC) studies and show the ability of MAB-A to specifically label a variety of non-small cell lung cancer types (FIG. 7A, Panels 1-8), breast cancer cells, prostate cancer cells, gastric cancer cells (FIG. 7B, Panels 1-6), and colon cancer cells (FIG. 7C, Panels 1-8) while the isotype control failed to specifically label any of these cancer cell types (FIG. 7A, FIG. 7B and FIG. 7C).

FIG. 8A and FIG. 8B present the results of cell staining studies and show that MAB-A binds to human ADAM9, and to a lesser extent, cynomolgus monkey ADAM9, transiently expressed on the surface of 293-FT and CHO-K cells (FIG. 8A and FIG. 8B, respectively).

FIG. 9A and FIG. 9B depict the amino acid sequences of the murine anti-ADAM9-VH Domain aligned with several humanized/optimized variants of MAB-A (FIG. 9A, SEQ ID NOs:7, 16, 17, 18, 19, 21, 22, 23 and 28) and the murine anti-ADAM9-VL Domain aligned with several humanized/optimized variants of MAB-A (FIG. 9B, SEQ ID NOs:11, 51, 52, 53 and 54). Positions substituted within the CDRs during the initial optimization are underlined as follows: potential deamidation and isomeration sites are indicated with a single underline, lysine residues are indicated with double underline, additional labile residues are indicated with a double dashed underline.

FIG. 10A and FIG. 10B present the ELISA binding curves of the ten selected optimized hMAB-A clones comprising CDR_{H}3 variants, the parental hMAB-A (2.2), and an isotype control antibody. FIG. 10A presents the binding curves for cynoADAM9 and FIG. 10B presents the binding curves for huADAM9.

### DETAILED DESCRIPTION OF THE INVENTION

The present invention is directed to molecules, such as monospecific antibodies and bispecific, trispecific or multispecific binding molecules, including diabodies, BITE® molecules, and antibodies that are capable of specifically binding to “Disintegrin and Metalloproteinase Domain-containing Protein 9” (“ADAM9”). The invention particularly concerns such binding molecules that are capable of exhibiting high affinity binding to human and non-human ADAM9. The invention further particularly relates to such molecules that are thereby cross-reactive with human ADAM9 and the ADAM9 of a non-human primate (e.g., a cynomolgus monkey). The invention additionally pertains to all such ADAM9-binding molecules that comprise a Light Chain Variable (VL) Domain and/or a Heavy Chain Variable (VH) Domain that has been humanized and/or deimmunized so as to exhibit reduced immunogenicity upon administration of such ADAM9-binding molecule to a recipient subject. The invention is also directed to pharmaceutical compositions that contain any of such ADAM9-binding molecules, and to methods involving the use of any of such ADAM9-binding molecules in the treatment of cancer and other diseases and conditions.

I. Antibodies and their Binding Domains

The antibodies of the present invention are immunoglobulin molecules capable of specific binding to a target, such as a carbohydrate, polynucleotide, lipid, polypeptide, etc., through at least one antigen recognition site, located in the Variable Domain of the immunoglobulin molecule. As used herein, the terms “antibody” and “antibodies” refer to monoclonal antibodies, multispecific antibodies, human antibodies, humanized antibodies, synthetic antibodies, chimeric antibodies, polyclonal antibodies, camelized antibodies, single-chain Fvs (scFv), single-chain antibodies, Fab fragments, F(ab′) fragments, disulfide-linked bispecific Fvs (sdFv), intrabodies, and epitope-binding fragments of any of the above. In particular, the term “antibody” includes immunoglobulin molecules and immunologically active fragments of immunoglobulin molecules, i.e., molecules that contain an epitope-binding site. Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass. The last few decades have seen a revival of interest in the therapeutic potential of antibodies, and antibodies have become one of the leading classes of biotechnology-derived drugs (Chan, C. E. et al. (2009) “The Use Of Antibodies In The Treatment Of Infectious Diseases,” Singapore Med. J. 50(7):663-666). In addition to their use in diagnostics, antibodies have been shown to be useful as therapeutic agents. Over 200 antibody-based drugs have been approved for use or are under development.

Antibodies are capable of “immunospecifically binding” to a polypeptide or protein or a non-protein molecule due to the presence on such molecule of a particular domain or moiety or conformation (an “epitope”). An epitope-containing molecule may have immunogenic activity, such that it elicits an antibody production response in an animal; such molecules are termed “antigens.” As used herein, an antibody, diabody or other epitope-binding molecule is said to “immunospecifically” bind a region of another molecule (i.e., an epitope) if it reacts or associates more frequently, more rapidly, with greater duration and/or with greater affinity with that epitope relative to alternative epitopes. For example, an antibody that immunospecifically binds to a viral epitope is an antibody that binds that viral epitope with greater affinity, avidity, more readily, and/or with greater duration than it immunospecifically binds to other viral epitopes or to non-viral epitopes. It is also understood by reading this definition that, for example, an antibody (or moiety or epitope) that immunospecifically binds to a first target may or may not specifically or preferentially bind to a second target. As such, “immunospecific binding” to a particular epitope does not necessarily require (although it can include) exclusive binding to that epitope. Generally, but not necessarily, reference to binding means “immunospecific” binding. Two molecules are said to be capable of binding to one another in a “physiospecific” manner, if such binding exhibits the specificity with which receptors bind to their respective ligands.

The term “monoclonal antibody” refers to a homogeneous antibody population wherein the monoclonal antibody is comprised of amino acids (naturally occurring or non-naturally occurring) that are involved in the selective binding of an antigen. Monoclonal antibodies are highly specific, being directed against a single epitope (or antigenic site). The term “monoclonal antibody” encompasses not only intact monoclonal antibodies and full-length monoclonal antibodies, but also fragments thereof (such as Fab, Fab′, F(ab′)₂ Fv), single-chain (scFv), mutants thereof, fusion proteins comprising an antibody portion, humanized monoclonal antibodies, chimeric monoclonal antibodies, and any other modified configuration of the immunoglobulin molecule that comprises an antigen recognition site of the required specificity and the ability to bind to an antigen. The term is not intended to be limited as regards to the source of the antibody or the manner in which it is made (e.g., by hybridoma, phage selection, recombinant expression, transgenic animals, etc.). The term includes whole immunoglobulins as well as the fragments etc. described above under the definition of “antibody.” Methods of making monoclonal antibodies are known in the art. One method which may be employed is the method of Kohler, G. et al. (1975) “Continuous Cultures Of Fused Cells Secreting Antibody Of Predefined Specificity,” Nature 256:495-497, or a modification thereof. Typically, monoclonal antibodies are developed in mice, rats or rabbits. The antibodies are produced by immunizing an animal with an immunogenic amount of cells, cell extracts, or protein preparations that contain the desired epitope. The immunogen can be, but is not limited to, primary cells, cultured cell lines, cancerous cells, proteins, peptides, nucleic acids, or tissue. Cells used for immunization may be cultured for a period of time (e.g., at least 24 hours) prior to their use as an immunogen. Cells may be used as immunogens by themselves or in combination with a non-denaturing adjuvant, such as Ribi (see, e.g., Jennings, V. M. (1995) “Review of Selected Adjuvants Used in Antibody Production,” ILAR J. 37(3):119-125). In general, cells should be kept intact and preferably viable when used as immunogens. Intact cells may allow antigens to be better detected than ruptured cells by the immunized animal. Use of denaturing or harsh adjuvants, e.g., Freund's adjuvant, may rupture cells and therefore is discouraged. The immunogen may be administered multiple times at periodic intervals such as, bi weekly, or weekly, or may be administered in such a way as to maintain viability in the animal (e.g., in a tissue recombinant). Alternatively, existing monoclonal antibodies and any other equivalent antibodies that are immunospecific for a desired pathogenic epitope can be sequenced and produced recombinantly by any means known in the art. In one embodiment, such an antibody is sequenced and the polynucleotide sequence is then cloned into a vector for expression or propagation. The sequence encoding the antibody of interest may be maintained in a vector in a host cell and the host cell can then be expanded and frozen for future use. The polynucleotide sequence of such antibodies may be used for genetic manipulation to generate the monospecific or multispecific (e.g., bispecific, trispecific and tetraspecific) molecules of the invention as well as an affinity optimized, a chimeric antibody, a humanized antibody, and/or a caninized antibody, to improve the affinity, or other characteristics of the antibody. The general principle in humanizing an antibody involves retaining the basic sequence of the antigen-binding portion of the antibody, while swapping the non-human remainder of the antibody with human antibody sequences.

Natural antibodies (such as natural IgG antibodies) are composed of two “Light Chains” complexed with two “Heavy Chains.” Each Light Chain contains a Variable Domain (“VL”) and a Constant Domain (“CL”). Each Heavy Chain contains a Variable Domain (“VH”), three Constant Domains (“CH1,” “CH2” and “CH3”), and a “Hinge” Region (“H”) located between the CH1 and CH2 Domains. The basic structural unit of naturally occurring immunoglobulins (e.g., IgG) is thus a tetramer having two light chains and two heavy chains, usually expressed as a glycoprotein of about 150,000 Da. The amino-terminal (“N-terminal”) portion of each chain includes a Variable Domain of about 100 to 110 or more amino acids primarily responsible for antigen recognition. The carboxy-terminal (“C-terminal”) portion of each chain defines a constant region, with light chains having a single Constant Domain and heavy chains usually having three Constant Domains and a Hinge Region. Thus, the structure of the light chains of an IgG molecule is n-VL-CL-c and the structure of the IgG heavy chains is n-VH-CH1-H-CH2-CH3-c (where n and c represent, respectively, the N-terminus and the C-terminus of the polypeptide). The Variable Domains of an IgG molecule consist of 1, 2, and most commonly 3, complementarity determining regions (“CDR”, i.e., CDR1, CDR2 and CDR3, respectively), which contain the residues in contact with epitope, and non-CDR segments, referred to as framework regions (“FR”), which in general maintain the structure and determine the positioning of the CDR regions so as to permit such contacting (although certain framework residues may also contact the epitope). Thus, the VL and VH Domains typically have the structure: n-FR1-CDR1-FR2-CDR2-FR3-CDR3-FR4-c (where “n” denotes the N-terminus and “c” denotes the C-terminus). Polypeptides that are (or may serve as) the first, second, third, and fourth FR of the Light Chain of an antibody are herein respectively designated as: FR_{L}1 Domain, FR_{L}2 Domain, FR_{L}3 Domain, and FR_{L}4 Domain. Similarly, polypeptides that are (or may serve as) the first, second, third and fourth FR of the Heavy Chain of an antibody are herein respectively designated as: FR_{H}1 Domain, FR_{H}2 Domain, FR_{H}3 Domain and FR_{H}4 Domain. Polypeptides that are (or may serve as) the first, second and third CDR of the Light Chain of an antibody are herein respectively designated as: CDR_{L}1 Domain, CDR_{L}2 Domain, and CDR_{L}3 Domain. Similarly, polypeptides that are (or may serve as) the first, second and third CDR of the Heavy Chain of an antibody are herein respectively designated as: CDR_{H}1 Domain, CDR_{H}2 Domain, and CDR_{H}3 Domain. Thus, the terms CDR_{L}1 Domain, CDR_{L}2 Domain, CDR_{L}3 Domain, CDR_{H}1 Domain, CDR_{H}2 Domain, and CDR_{H}3 Domain are directed to polypeptides that when incorporated into a protein cause that protein to be able to bind to a specific epitope regardless of whether such protein is an antibody having light and heavy chains or is a diabody or a single-chain binding molecule (e.g., an scFv, a BITE®, etc.), or is another type of protein.

Accordingly, as used herein, the term “epitope-binding fragment” means a fragment of an antibody capable of immunospecifically binding to an epitope, and the term “epitope-binding site” refers to a portion of a molecule comprising an epitope-binding fragment. An epitope-binding fragment may contain any 1, 2, 3, 4, or 5 the CDR Domains of an antibody, or may contain all 6 of the CDR Domains of an antibody and, although capable of immunospecifically binding to such epitope, may exhibit an immunospecificity, affinity or selectivity toward such epitope that differs from that of such antibody. Preferably, however, an epitope-binding fragment will contain all 6 of the CDR Domains of such antibody. An epitope-binding fragment of an antibody may be a single polypeptide chain (e.g., an scFv), or may comprise two or more polypeptide chains, each having an amino terminus and a carboxy terminus (e.g., a diabody, a Fab fragment, an Fab₂ fragment, etc.). Unless specifically noted, the order of domains of the protein molecules described herein is in the “N-terminal to C-terminal” direction.

The invention particularly encompasses single-chain Variable Domain fragments (“scFv”) comprising an anti-ADAM9-VL and/or VH Domain of the invention as well as multispecific binding molecules comprising such anti-ADAM9-VL and/or VH Domains. Single-chain Variable Domain fragments comprise VL and VH Domains that are linked together using a short “Linker” peptide. Such Linkers can be modified to provide additional functions, such as to permit the attachment of a drug or to permit attachment to a solid support. The single-chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

The invention also particularly encompasses the CDR_{H}1, CDR_{H}2, CDR_{H}3, CDR_{L}1, CDR_{L}2, and CDR_{L}3 Domains of humanized variants of the anti-ADAM9 antibodies of the invention, as well as VL Domains that contain any 1, 2, or 3 of such CDR_{L}s and VH Domains that contain any 1, 2, or 3 of such CDR_{H}s, as well as multispecific-binding molecules comprising the same. The term “humanized” antibody refers to a chimeric molecule having an epitope-binding site of an immunoglobulin from a non-human species and a remaining immunoglobulin structure that is based upon the structure and/or sequence of a human immunoglobulin. Humanized antibodies are generally prepared using recombinant techniques. The anti-ADAM9 antibodies of the present invention include humanized, chimeric or caninized variants of an antibody that is designated herein as “MAB-A.” The polynucleotide sequences that encode the Variable Domains of MAB-A may be used for genetic manipulation to generate MAB-A derivatives possessing improved or altered characteristics (e.g., affinity, cross-reactivity, specificity, etc.). The general principle in humanizing an antibody involves retaining the basic sequence of the epitope-binding portion of the antibody, while swapping the non-human remainder of the antibody with human antibody sequences. There are four general steps to humanize a monoclonal antibody. These are: (1) determining the nucleotide and predicted amino acid sequence of the starting antibody light and heavy variable domains; (2) designing the humanized antibody or caninized antibody, i.e., deciding which antibody framework region to use during the humanizing or canonizing process; (3) employing the actual humanizing or caninizing methodologies/techniques; and (4) transfecting and expressing the humanized antibody. See, for example, U.S. Pat. Nos. 4,816,567; 5,807,715; 5,866,692; and 6,331,415. The term “optimized” antibody refers to an antibody having at least one amino acid which is different from the parent antibody in at least one complementarity determining region (CDR) in the light or heavy chain variable region, which confers a higher binding affinity, (e.g., a 2-fold or more fold) higher binding affinity, to human ADAM9 and/or cynomolgus monkey ADAM9 as compared to the parental antibody. It will be understood from the teaching provided herein that the antibodies of the invention may be humanized, optimized, or both humanized and optimized.

The epitope-binding site may comprise either a complete Variable Domain fused to one or more Constant Domains or only the CDRs of such Variable Domain grafted to appropriate framework regions. Epitope-binding sites may be wild-type or may be modified by one or more amino acid substitutions, insertions or deletions. Such action partially or completely eliminates the ability of the Constant Region to serve as an immunogen in recipients (e.g., human individuals), however, the possibility of an immune response to the foreign Variable Domain remains (LoBuglio, A. F. et al. (1989) “Mouse/Human Chimeric Monoclonal Antibody In Man: Kinetics And Immune Response,” Proc. Natl. Acad. Sci. (U.S.A.) 86:4220-4224). Another approach focuses not only on providing human-derived constant regions, but on modifying the Variable Domains as well so as to reshape them as closely as possible to a form found in human immunoglobulins. It is known that the Variable Domains of both the Heavy and Light Chains of antibodies contain three CDRs which vary in response to the antigens in question and determine binding capability, flanked by the four framework regions, which are relatively conserved in a given species and which putatively provide a scaffolding for the CDRs. When non-human antibodies are prepared with respect to a particular antigen, the variable domains can be “reshaped” or “humanized” by grafting CDRs derived from non-human antibody on the FRs present in the human antibody to be modified. Application of this approach to various antibodies has been reported by Sato, K. et al. (1993) Cancer Res 53:851-856. Riechmann, L. et al. (1988) “Reshaping Human Antibodies for Therapy,” Nature 332:323-327; Verhoeyen, M. et al. (1988) “Reshaping Human Antibodies: Grafting An Antilysozyme Activity,” Science 239:1534-1536; Kettleborough, C. A. et al. (1991) “Humanization Of A Mouse Monoclonal Antibody By CDR-Grafting: The Importance Of Framework Residues On Loop Conformation,” Protein Engineering 4:773-3783; Maeda, H. et al. (1991) “Construction Of Reshaped Human Antibodies With HIV-Neutralizing Activity,” Human Antibodies Hybridoma 2:124-134; Gorman, S. D. et al. (1991) “Reshaping A Therapeutic CD4 Antibody,” Proc. Natl. Acad. Sci. (U.S.A.) 88:4181-4185; Tempest, P. R. et al. (1991) “Reshaping A Human Monoclonal Antibody To Inhibit Human Respiratory Syncytial Virus Infection in vivo,” Bio/Technology 9:266-271; Co, M. S. et al. (1991) “Humanized Antibodies For Antiviral Therapy,” Proc. Natl. Acad. Sci. (U.S.A.) 88:2869-2873; Carter, P. et al. (1992) “Humanization Of An Anti-p185her2 Antibody For Human Cancer Therapy,” Proc. Natl. Acad. Sci. (U.S.A.) 89:4285-4289; and Co, M. S. et al. (1992) “Chimeric And Humanized Antibodies With Specificity For The CD33 Antigen,” J. Immunol. 148:1149-1154. In some embodiments, humanized antibodies preserve all CDR sequences (for example, a humanized murine antibody which contains all six of the CDRs present in the murine antibody). In other embodiments, humanized antibodies have one or more CDRs (one, two, three, four, five, or six) that differ in sequence relative to the CDRs of the original antibody.

A number of humanized antibody molecules comprising an epitope-binding site derived from a non-human immunoglobulin have been described, including chimeric antibodies having rodent or modified rodent Variable Domain and their associated complementarity determining regions (CDRs) fused to human constant domains (see, for example, Winter et al. (1991) “Man-made Antibodies,” Nature 349:293-299; Lobuglio et al. (1989) “Mouse/Human Chimeric Monoclonal Antibody In Man: Kinetics And Immune Response,” Proc. Natl. Acad. Sci. (U.S.A.) 86:4220-4224; Shaw et al. (1987) “Characterization Of A Mouse/Human Chimeric Monoclonal Antibody (17-1A) To A Colon Cancer Tumor-Associated Antigen,” J. Immunol. 138:4534-4538; and Brown et al. (1987) “Tumor-Specific Genetically Engineered Murine/Human Chimeric Monoclonal Antibody,” Cancer Res. 47:3577-3583). Other references describe rodent CDRs grafted into a human supporting framework region (FR) prior to fusion with an appropriate human antibody Constant Domain (see, for example, Riechmann, L. et al. (1988) “Reshaping Human Antibodies for Therapy,” Nature 332:323-327; Verhoeyen, M. et al. (1988) “Reshaping Human Antibodies: Grafting An Antilysozyme Activity,” Science 239:1534-1536; and Jones et al. (1986) “Replacing The Complementarity-Determining Regions In A Human Antibody With Those From A Mouse,” Nature 321:522-525). Another reference describes rodent CDRs supported by recombinantly veneered rodent framework regions (see, for example, European Patent Publication No. 519,596). These “humanized” molecules are designed to minimize unwanted immunological response towards rodent anti-human antibody molecules, which limits the duration and effectiveness of therapeutic applications of those moieties in human recipients. Other methods of humanizing antibodies that may also be utilized are disclosed by Daugherty et al. (1991) “Polymerase Chain Reaction Facilitates The Cloning, CDR-Grafting, And Rapid Expression Of A Murine Monoclonal Antibody Directed Against The CD18 Component Of Leukocyte Integrins,” Nucl. Acids Res. 19:2471-2476 and in U.S. Pat. Nos. 6,180,377; 6,054,297; 5,997,867; and 5,866,692.

### II. Fcγ Receptors (FcγRs)

The CH2 and CH3 Domains of the two Heavy Chains of an antibody interact to form an “Fc Region,” which is a domain that is recognized by cellular “Fc Receptors,” including but not limited to Fc gamma Receptors (“FcγRs”). As used herein, the term “Fc Region” is used to define the C-terminal region of an IgG Heavy Chain that comprises the CH2 and CH3 Domains of that chain. An Fc Region is said to be of a particular IgG isotype, class or subclass if its amino acid sequence is most homologous to that isotype, relative to other IgG isotypes.

The amino acid sequence of the CH2-CH3 Domain of an exemplary human IgG1 is (SEQ ID NO:1):

```
231 240 250 260 270 280
APELLGGPSV FLFPPKPKDT LMISRTPEVT CVVVDVSHED PEVKFNWYVD
                  290 300 310 320 330
GVEVHNAKTK PREEQYNSTY RVVSVLTVLH QDWLNGKEYK CKVSNKALPA
                  340 350 360 370 380
PIEKTISKAK GQPREPQVYT LPPSREEMTK NQVSLTCLVK GFYPSDIAVE
                  390 400 410 420 430
WESNGQPENN YKTTPPVLDS DGSFFLYSKL TVDKSRWQQG NVFSCSVMHE
                  440 447
ALHNHYTQKS LSLSPGX
```

as numbered by the EU index as set forth in Kabat, wherein X is a lysine (K) or is absent.

The amino acid sequence of the CH2-CH3 Domain of an exemplary human IgG2 is (SEQ ID NO:2):

```
231 240 250 260 270 280
APPVA-GPSV FLFPPKPKDT LMISRTPEVT CVVVDVSHED PEVQFNWYVD
                  290 300 310 320 330
GVEVHNAKTK PREEQFNSTF RVVSVLTVVH QDWLNGKEYK CKVSNKGLPA
                  340 350 360 370 380
PIEKTISKTK GQPREPQVYT LPPSREEMTK NQVSLTCLVK GFYPSDISVE
                  390 400 410 420 430
WESNGQPENN YKTTPPMLDS DGSFFLYSKL TVDKSRWQQG NVFSCSVMHE
                  440 447
ALHNHYTQKS LSLSPGX
```

as numbered by the EU index as set forth in Kabat, wherein X is a lysine (K) or is absent.

The amino acid sequence of the CH2-CH3 Domain of an exemplary human IgG3 is (SEQ ID NO:3):

```
231 240 250 260 270 280
APELLGGPSV FLFPPKPKDT LMISRTPEVT CVVVDVSHED PEVQFKWYVD
                  290 300 310 320 330
GVEVHNAKTK PREEQYNSTF RVVSVLTVLH QDWLNGKEYK CKVSNKALPA
                  340 350 360 370 380
PIEKTISKTK GQPREPQVYT LPPSREEMTK NQVSLTCLVK GFYPSDIAVE
                  390 400 410 420 430
WESSGQPENN YNTTPPMLDS DGSFFLYSKL TVDKSRWQQG NIFSCSVMHE
                  440 447
ALHNRFTQKS LSLSPGX
```

as numbered by the EU index as set forth in Kabat, wherein X is a lysine (K) or is absent.

The amino acid sequence of the CH2-CH3 Domain of an exemplary human IgG4 is (SEQ ID NO:4):

```
231 240 250 260 270 280
APEFLGGPSV FLFPPKPKDT LMISRTPEVT CVVVDVSQED PEVQFNWYVD
                  290 300 310 320 330
GVEVHNAKTK PREEQFNSTY RVVSVLTVLH QDWLNGKEYK CKVSNKGLPS
                  340 350 360 370 380
SIEKTISKAK GQPREPQVYT LPPSQEEMTK NQVSLTCLVK GFYPSDIAVE
                  390 400 410 420 430
WESNGQPENN YKTTPPVLDS DGSFFLYSRL TVDKSRWQEG NVFSCSVMHE
                  440 447
ALHNHYTQKS LSLSLGX
```

as numbered by the EU index as set forth in Kabat, wherein X is a lysine (K) or is absent.

Throughout the present specification, the numbering of the residues in the constant region of an IgG heavy chain is that of the EU index as in Kabat et al., SEQUENCES OF PROTEINS OF IMMUNOLOGICAL INTEREST, 5^{th} Ed. Public Health Service, NH1, MD (1991) (“Kabat”), expressly incorporated herein by reference. The term “the EU index as set forth in Kabat” refers to the numbering of the Constant Domains of human IgG1 EU antibody provided in Kabat. Amino acids from the Variable Domains of the mature heavy and light chains of immunoglobulins are designated by the position of an amino acid in the chain. Kabat described numerous amino acid sequences for antibodies, identified an amino acid consensus sequence for each subgroup, and assigned a residue number to each amino acid, and the CDRs are identified as defined by Kabat (it will be understood that CDR_{H}1 as defined by Chothia, C. & Lesk, A. M. ((1987) “Canonical structures for the hypervariable regions of immunoglobulins,” J. Mol. Biol. 196:901-917) begins five residues earlier). Kabat's numbering scheme is extendible to antibodies not included in his compendium by aligning the antibody in question with one of the consensus sequences in Kabat by reference to conserved amino acids. This method for assigning residue numbers has become standard in the field and readily identifies amino acids at equivalent positions in different antibodies, including chimeric or humanized variants. For example, an amino acid at position 50 of a human antibody light chain occupies the equivalent position to an amino acid at position 50 of a mouse antibody light chain.

Polymorphisms have been observed at a number of different positions within antibody constant regions (e.g., Fc positions, including but not limited to positions 270, 272, 312, 315, 356, and 358 as numbered by the EU index as set forth in Kabat), and thus slight differences between the presented sequence and sequences in the prior art can exist. Polymorphic forms of human immunoglobulins have been well-characterized. At present, 18 Gm allotypes are known: G1m (1, 2, 3, 17) or G1m (a, x, f, z), G2m (23) or G2m (n), G3m (5, 6, 10, 11, 13, 14, 15, 16, 21, 24, 26, 27, 28) or G3m (b1, c3, b3, b0, b3, b4, s, t, g1, c5, u, v, g5) (Lefranc, et al., “The Human IgG Subclasses: Molecular Analysis of Structure, Function And Regulation.” Pergamon, Oxford, pp. 43-78 (1990); Lefranc, G. et al., 1979, Hum. Genet.: 50, 199-211). It is specifically contemplated that the antibodies of the present invention may incorporate any allotype, isoallotype, or haplotype of any immunoglobulin gene, and are not limited to the allotype, isoallotype or haplotype of the sequences provided herein. Furthermore, in some expression systems the C-terminal amino acid residue (bolded above) of the CH3 Domain may be post-translationally removed. Accordingly, the C-terminal residue of the CH3 Domain is an optional amino acid residue in the ADAM9-binding molecules of the invention. Specifically encompassed by the instant invention are ADAM9-binding molecules lacking the C-terminal residue of the CH3 Domain. Also specifically encompassed by the instant invention are such constructs comprising the C-terminal lysine residue of the CH3 Domain.

As stated above, the Fc Region of natural IgG antibodies is capable of binding to cellular Fc gamma Receptors (FcγRs). Such binding results in the transduction of activating or inhibitory signals to the immune system. The ability of such binding to result in diametrically opposing functions reflects structural differences among the different FcγRs, and in particular reflects whether the bound FcγR possesses an Immunoreceptor Tyrosine-Based Activation Motif (“ITAM”) or an Immunoreceptor Tyrosine-Based Inhibitory Motif (“ITIM”). The recruitment of different cytoplasmic enzymes to these structures dictates the outcome of the FcγR-mediated cellular responses. ITAM-containing FcγRs include FcγRI, FcγRIIA, FcγRIIIA, and activate the immune system when bound to Fc Regions (e.g., aggregated Fc Regions present in an immune complex). FcγRIIB is the only currently known natural ITIM-containing FcγR; it acts to dampen or inhibit the immune system when bound to aggregated Fc Regions. Human neutrophils express the FcγRIIA gene. FcγRIIA clustering via immune complexes or specific antibody cross-linking serves to aggregate ITAMs with receptor-associated kinases which facilitate ITAM phosphorylation. ITAM phosphorylation serves as a docking site for Syk kinase, the activation of which results in the activation of downstream substrates (e.g., PI₃K). Cellular activation leads to release of pro-inflammatory mediators. The FcγRIIB gene is expressed on B lymphocytes; its extracellular domain is 96% identical to FcγRIIA and binds IgG complexes in an indistinguishable manner. The presence of an ITIM in the cytoplasmic domain of FcγRIIB defines this inhibitory subclass of FcγR. Recently the molecular basis of this inhibition was established. When co-ligated along with an activating FcγR, the ITIM in FcγRIIB becomes phosphorylated and attracts the SH2 domain of the inositol polyphosphate 5′-phosphatase (SHIP), which hydrolyzes phosphoinositol messengers released as a consequence of ITAM-containing FcγR-mediated tyrosine kinase activation, consequently preventing the influx of intracellular Ca⁺⁺. Thus, cross-linking of FcγRIIB dampens the activating response to FcγR ligation and inhibits cellular responsiveness. B-cell activation, B-cell proliferation and antibody secretion is thus aborted.

### III. Bispecific Antibodies, Multispecific Diabodies and DART® Diabodies

The ability of an antibody to bind an epitope of an antigen depends upon the presence and amino acid sequence of the antibody's VL and VH Domains. Interaction of an antibody's Light Chain and Heavy Chain and, in particular, interaction of its VL and VH Domains forms one of the two epitope-binding sites of a natural antibody, such as an IgG. Natural antibodies are capable of binding to only one epitope species (i.e., they are monospecific), although they can bind multiple copies of that epitope species (i.e., exhibiting bivalency or multivalency).

The functionality of antibodies can be enhanced by generating multispecific antibody-based molecules that can simultaneously bind two separate and distinct antigens (or different epitopes of the same antigen) and/or by generating antibody-based molecule having higher valency (i.e., more than two binding sites) for the same epitope and/or antigen.

In order to provide molecules having greater capability than natural antibodies, a wide variety of recombinant bispecific antibody formats have been developed (see, e.g., PCT Publication Nos. WO 2008/003116, WO 2009/132876, WO 2008/003103, WO 2007/146968, WO 2009/018386, WO 2012/009544, WO 2013/070565), most of which use linker peptides either to fuse a further epitope-binding fragment (e.g., an scFv, VL, VH, etc.) to, or within the antibody core (IgA, IgD, IgE, IgG or IgM), or to fuse multiple epitope-binding fragments (e.g., two Fab fragments or scFvs). Alternative formats use linker peptides to fuse an epitope-binding fragment (e.g., an scFv, VL, VH, etc.) to a dimerization domain such as the CH2-CH3 Domain or alternative polypeptides (see, e.g., PCT Publication Nos. WO 2005/070966, WO 2006/107786A WO 2006/107617A, WO 2007/046893). PCT Publication Nos. WO 2013/174873, WO 2011/133886 and WO 2010/136172 disclose a trispecific antibody in which the CL and CH1 Domains are switched from their respective natural positions and the VL and VH Domains have been diversified (see, e.g., PCT Publication Nos. WO 2008/027236; WO 2010/108127) to allow them to bind to more than one antigen. PCT Publication Nos. WO 2013/163427 and WO 2013/119903 disclose modifying the CH2 Domain to contain a fusion protein adduct comprising a binding domain. PCT Publication Nos. WO 2010/028797, WO 2010/028796 and WO 2010/028795 disclose recombinant antibodies whose Fc Regions have been replaced with additional VL and VH Domains, so as to form trivalent binding molecules. PCT Publication Nos. WO 2003/025018 and WO 2003/012069 disclose recombinant diabodies whose individual chains contain scFv Domains. PCT Publication Nos. WO 2013/006544 discloses multivalent Fab molecules that are synthesized as a single polypeptide chain and then subjected to proteolysis to yield heterodimeric structures. PCT Publication Nos. WO 2014/022540, WO 2013/003652, WO 2012/162583, WO 2012/156430, WO 2011/086091, WO 2008/024188, WO 2007/024715, WO 2007/075270, WO 1998/002463, WO 1992/022583 and WO 1991/003493 disclose adding additional binding domains or functional groups to an antibody or an antibody portion (e.g., adding a diabody to the antibody's light chain, or adding additional VL and VH Domains to the antibody's light and heavy chains, or adding a heterologous fusion protein or chaining multiple Fab Domains to one another).

The design of a diabody is based on the antibody derivative known as a single-chain Variable Domain fragment (scFv). Such molecules are made by linking Light and/or Heavy Chain Variable Domains using a short linking peptide. Bird, R. E. et al. (1988) (“Single-Chain Antigen-Binding Proteins,” Science 242:423-426) describes examples of linking peptides which bridge approximately 3.5 nm between the carboxy terminus of one Variable Domain and the amino terminus of the other Variable Domain. Linkers of other sequences have been designed and used (Bird et al. (1988) “Single-Chain Antigen-Binding Proteins,” Science 242:423-426). Linkers can in turn be modified for additional functions, such as attachment of drugs or attachment to solid supports. The single-chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

The art has noted the capability to produce diabodies that differ from such natural antibodies in being capable of binding two or more different epitope species (i.e., exhibiting bispecificity or multispecificity in addition to bivalency or multivalency) (see, e.g., Holliger, P. et al. (1993) “‘Diabodies’: Small Bivalent And Bispecific Antibody Fragments,” Proc. Natl. Acad. Sci. (U.S.A.) 90:6444-6448; US 2004/0058400 (Hollinger et al.); US 2004/0220388/WO 02/02781 (Mertens et al.); Alt et al. (1999) FEBS Lett. 454(1-2):90-94; Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672; WO 02/02781 (Mertens et al.); Olafsen, T. et al. (2004) “Covalent Disulfide-Linked Anti-CEA Diabody Allows Site-Specific Conjugation And Radiolabeling For Tumor Targeting Applications,” Protein Eng. Des. Sel. 17(1):21-27; Wu, A. et al. (2001) “Multimerization Of A Chimeric Anti-CD20 Single Chain Fv-Fv Fusion Protein Is Mediated Through Variable Domain Exchange,” Protein Engineering 14(2):1025-1033; Asano et al. (2004) “A Diabody For Cancer Immunotherapy And Its Functional Enhancement By Fusion Of Human Fc Domain,” Abstract 3P-683, J. Biochem. 76(8):992; Takemura, S. et al. (2000) “Construction Of A Diabody (Small Recombinant Bispecific Antibody) Using A Refolding System,” Protein Eng. 13(8):583-588; Baeuerle, P. A. et al. (2009) “Bispecific T-Cell Engaging Antibodies For Cancer Therapy,” Cancer Res. 69(12):4941-4944).

The provision of bispecific binding molecules (e.g., non-monospecific diabodies) provides a significant advantage over antibodies, including but not limited to, a “trans” binding capability sufficient to co-ligate and/or co-localize different cells that express different epitopes and/or a “cis” binding capability sufficient to co-ligate and/or co-localize different molecules expressed by the same cell. Bispecific binding molecules (e.g., non-monospecific diabodies) thus have wide-ranging applications including therapy and immunodiagnosis. Bispecificity allows for great flexibility in the design and engineering of the diabody in various applications, providing enhanced avidity to multimeric antigens, the cross-linking of differing antigens, and directed targeting to specific cell types relying on the presence of both target antigens. Due to their increased valency, low dissociation rates and rapid clearance from the circulation (for diabodies of small size, at or below −50 kDa), diabody molecules known in the art have also shown particular use in the field of tumor imaging (Fitzgerald et al. (1997) “Improved Tumour Targeting By Disulphide Stabilized Diabodies Expressed In Pichia pastoris,” Protein Eng. 10:1221-1225).

The ability to produce bispecific diabodies has led to their use (in “trans”) to co-ligate two cells together, for example, by co-ligating receptors that are present on the surface of different cells (e.g., cross-linking cytotoxic T-cells to tumor cells) (Staerz et al. (1985) “Hybrid Antibodies Can Target Sites For Attack By T Cells,” Nature 314:628-631; Holliger et al. (1996) “Specific Killing Of Lymphoma Cells By Cytotoxic T-Cells Mediated By A Bispecific Diabody,” Protein Eng. 9:299-305; and Marvin et al. (2005) “Recombinant Approaches To IgG-Like Bispecific Antibodies,” Acta Pharmacol. Sin. 26:649-658). Alternatively (or additionally), bispecific (or tri- or multispecific) diabodies can be used (in “cis”) to co-ligate molecules, such as receptors, etc., that are present on the surface of the same cell. Co-ligation of different cells and/or receptors is useful to modulate effector functions and/or immune cell signaling. Multispecific molecules (e.g., bispecific diabodies) comprising epitope-binding sites may be directed to a surface determinant of any immune cell such as CD2, CD3, CD8, CD16, T-Cell Receptor (TCR), NKG2D, etc., which are expressed on T lymphocytes, Natural Killer (NK) cells, Antigen-Presenting Cells or other mononuclear cells. In particular, epitope-binding sites directed to a cell surface receptor that is present on immune effector cells, are useful in the generation of multispecific binding molecules capable of mediating redirected cell killing.

However, the above advantages come at a salient cost. The formation of such non-monospecific diabodies requires the successful assembly of two or more distinct and different polypeptides (i.e., such formation requires that the diabodies be formed through the heterodimerization of different polypeptide chain species). This fact is in contrast to monospecific diabodies, which are formed through the homodimerization of identical polypeptide chains. Because at least two dissimilar polypeptides (i.e., two polypeptide species) must be provided in order to form a non-monospecific diabody, and because homodimerization of such polypeptides leads to inactive molecules (Takemura, S. et al. (2000) “Construction Of A Diabody (Small Recombinant Bispecific Antibody) Using A Refolding System,” Protein Eng. 13(8):583-588), the production of such polypeptides must be accomplished in such a way as to prevent covalent bonding between polypeptides of the same species (i.e., so as to prevent homodimerization) (Takemura, S. et al. (2000) “Construction Of A Diabody (Small Recombinant Bispecific Antibody) Using A Refolding System,” Protein Eng. 13(8):583-588). The art has therefore taught the non-covalent association of such polypeptides (see, e.g., Olafsen et al. (2004) “Covalent Disulfide-Linked Anti-CEA Diabody Allows Site-Specific Conjugation And Radiolabeling For Tumor Targeting Applications,” Prot. Engr. Des. Sel. 17:21-27; Asano et al. (2004) “A Diabody For Cancer Immunotherapy And Its Functional Enhancement By Fusion Of Human Fc Domain,” Abstract 3P-683, J. Biochem. 76(8):992; Takemura, S. et al. (2000) “Construction Of A Diabody (Small Recombinant Bispecific Antibody) Using A Refolding System,” Protein Eng. 13(8):583-588; and Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672).

However, the art has recognized that bispecific diabodies composed of non-covalently associated polypeptides are unstable and readily dissociate into non-functional monomers (see, e.g., Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672).

In the face of this challenge, the art has succeeded in developing stable, covalently bonded heterodimeric non-monospecific diabodies, termed DART® diabodies; see, e.g., U.S. Pat. Nos. 9,296,816 and 9,284,375 and US Patent Publication Nos. 2015/0175697; 2014/0255407; 2014/0099318; 2013/0295121; WO 2012/018687; WO 2012/162068; 2010/0174053; WO 2010/080538; 2009/0060910; 2007-0004909; European Patent Publication Nos. EP 2714079; EP 2601216; EP 2376109; EP 2158221; EP 1868650; and PCT Publication Nos. WO 2012/162068; WO 2012/018687; WO 2010/080538; WO 2006/113665; and Sloan, D. D. et al. (2015) “Targeting HIV Reservoir in Infected CD4 T Cells by Dual-Affinity Re-targeting Molecules (DARTs) that Bind HIV Envelope and Recruit Cytotoxic T Cells,” PLoS Pathog. 11(11):e1005233. doi: 10.1371/journal.ppat.1005233; Al Hussaini, M. et al. (2015) “Targeting CD123 In AML Using A T-Cell Directed Dual-Affinity Re-Targeting (DART®) Platform,” Blood pii: blood-2014-05-575704; Chichili, G. R. et al. (2015) “A CD3×CD123 Bispecific DART For Redirecting Host T Cells To Myelogenous Leukemia: Preclinical Activity And Safety In Nonhuman Primates,” Sci. Transl. Med. 7(289):289ra82; Moore, P. A. et al. (2011) “Application Of Dual Affinity Retargeting Molecules To Achieve Optimal Redirected T-Cell Killing Of B-Cell Lymphoma,” Blood 117(17):4542-4551; Veri, M. C. et al. (2010) “Therapeutic Control Of B Cell Activation Via Recruitment Of Fcgamma Receptor IIb (CD32B) Inhibitory Function With A Novel Bispecific Antibody Scaffold,” Arthritis Rheum. 62(7):1933-1943; and Johnson, S. et al. (2010) “Effector Cell Recruitment With Novel Fv-Based Dual-Affinity Re-Targeting Protein Leads To Potent Tumor Cytolysis And in vivo B-Cell Depletion,” J. Mol. Biol. 399(3):436-449). Such diabodies comprise two or more covalently complexed polypeptides and involve engineering one or more cysteine residues into each of the employed polypeptide species that permit disulfide bonds to form and thereby covalently bond one or more pairs of such polypeptide chains to one another. For example, the addition of a cysteine residue to the C-terminus of such constructs has been shown to allow disulfide bonding between the involved polypeptide chains, stabilizing the resulting diabody without interfering with the diabody's binding characteristics. Such molecules can be made to be bispecific (or multispecific) and thus may be made to co-ligate two or more molecules. Such co-ligation permits one to provide an enhanced immunotherapy. Additionally, because the individual polypeptide chains of such molecules form a covalently bonded complex, the molecules exhibit far greater stability than diabodies involving non-covalently bonded polypeptide chains.

Recently, trivalent and multivalent molecules incorporating two diabody-type binding domains and one non-diabody-type domain and an Fc Region have been described (see, e.g., PCT Publication Nos. WO 2015/184207 and WO 2015/184203). Such binding molecules may be utilized to generate monospecific, bispecific or trispecific molecules. The ability to bind three different epitopes provides enhanced capabilities.

Alternative constructs are known in the art for applications where a tetravalent molecule is desirable but an Fc is not required, including, but not limited to, tetravalent tandem antibodies, also referred to as “TandAbs” (see, e.g. US Patent Publication Nos. 2005-0079170, 2007-0031436, 2010-0099853, 2011-020667 2013-0189263; European Patent Publication Nos. EP 1078004, EP 2371866, EP 2361936 and EP 1293514; PCT Publication Nos. WO 1999/057150, WO 2003/025018, and WO 2013/013700) which are formed by the homo-dimerization of two identical polypeptide chains, each possessing a VH1, VL2, VH2, and VL2 Domain.

### IV. ADAM9

A representative human ADAM9 polypeptide (NCBI Sequence NP_003807, including a 28 amino acid residue signal sequence, shown underlined) has the amino acid sequence (SEQ ID NO:5):

MGSGARFPSG TLRVRWLLLL GLVGPVLGAA RPGFQQTSHL
SSYEIITPWR LTRERREAPR PYSKQVSYVI QAEGKEHIIH
LERNKDLLPE DFVVYTYNKE GTLITDHPNI QNHCHYRGYV
EGVHNSSIAL SDCFGLRGLL HLENASYGIE PLQNSSHFEH
IIYRMDDVYK EPLKCGVSNK DIEKETAKDE EEEPPSMTQL
LRRRRAVLPQ TRYVELFIVV DKERYDMMGR NQTAVREEMI
LLANYLDSMY IMLNIRIVLV GLEIWTNGNL INIVGGAGDV
LGNPVQWREK FLITRRRHDS AQLVLKKGFG GTAGMAPVGT
VCSRSHAGGI NVFGQITVET FASIVAHELG HNLGMNHDDG
RDCSCGAKSC IMNSGASGSR NFSSCSAEDF EKLTLNKGGN
CLLNIPKPDE AYSAPSCGNK LVDAGEECDC GTPKECELDP
CCEGSTCKLK SFAECAYGDC CKDCRFLPGG TLCRGKTSEC
DVPEYCNGSS QFCQPDVFIQ NGYPCQNNKA YCYNGMCQYY
DAQCQVIFGS KAKAAPKDCF IEVNSKGDRF GNCGFSGNEY
KKCATGNALC GKLQCENVQE IPVFGIVPAI IQTPSRGTKC
WGVDFQLGSD VPDPGMVNEG TKCGAGKICR NFQCVDASVL
NYDCDVQKKC HGHGVCNSNK NCHCENGWAP PNCETKGYGG
SVDSGPTYNE MNTALRDGLL VFFFLIVPLI VCAIFIFIKR
DQLWRSYFRK KRSQTYESDG KNQANPSRQP GSVPRHVSPV
TPPREVPIYA NRFAVPTYAA KQPQQFPSRP PPPQPKVSSQ
GNLIPARPAP APPLYSSLT

Of the 819 amino acid residues of ADAM9 (SEQ ID NO:5), residues 1-28 are a signal sequence, residues 29-697 are the Extracellular Domain, residues 698-718 are the Transmembrane Domain, and residues 719-819 are the Intracellular Domain. Three structural domains are located within the Extracellular Domain: a Reprolysin (M12B) Family Zinc Metalloprotease Domain (at approximately residues 212-406); a Disintegrin Domain (at approximately residues 423-497); and an EGF-like Domain (at approximately residues 644-697). A number of post-translational modifications and isoforms have been identified and the protein is proteolytically cleaved in the trans-Golgi network before it reaches the plasma membrane to generate a mature protein. The removal of the pro-domain occurs via cleavage at two different sites. Processed most likely by a pro-protein convertase such as furin, at the boundary between the pro-domain and the catalytic domain (Arg-205/Ala-206). An additional upstream cleavage pro-protein convertase site (Arg-56/Glu-57) has an important role in the activation of ADAM9.

A representative cynomolgus monkey ADAM9 polypeptide (NCBI Sequence XM_005563126.2, including a possible 28 amino acid residue signal sequence, shown underlined) has the amino acid sequence (SEQ ID NO:6):

MGSGVGSPSG TLRVRWLLLL CLVGPVLGAA RPGFQQTSHL
SSYEIITPWR LTRERREAPR PYSKQVSYLI QAEGKEHIIH
LERNKDLLPE DFVVYTYNKE GTVITDHPNI QNHCHFRGYV
EGVYNSSVAL SNCFGLRGLL HLENASYGIE PLQNSSHFEH
IIYRMDDVHK EPLKCGVSNK DIEKETTKDE EEEPPSMTQL
LRRRRAVLPQ TRYVELFIVV DKERYDMMGR NQTAVREEMI
LLANYLDSMY IMLNIRIVLV GLEIWTNGNL INIAGGAGDV
LGNPVQWREK FLITRRRHDS AQLVLKKGFG GTAGMAPVGT
VCSRSHAGGI NVFGHITVET FASIVAHELG HNLGMNHDDG
RDCSCGAKSC IMNSGASGSR NFSSCSAEDF EKLTLNKGGN
CLLNIPKPDE AYSAPSCGNK LVDAGEECDC GTPKECELDP
CCEGSTCKLK SFAECAYGDC CKDCRFLPGG TLCRGKTSEC
DVPEYCNGSS QFCQPDVFIQ NGYPCQNNKA YCYNGMCQYY
DAQCQVIFGS KAKAAPKDCF IEVNSKGDRF GNCGFSGNEY
KKCATGNALC GKLQCENVQE IPVFGIVPAI IQTPSRGTKC
WGVDFQLGSD VPDPGMVNEG TKCGADKICR NFQCVDASVL
NYDCDIQKKC HGHGVCNSNK NCHCENGWAP PNCETKGYGG
SVDSGPTYNE MNTALRDGLL VFFFLIVPLI VCAIFIFIKR
DQLWRRYFRK KRSQTYESDG KNQANPSRQP VSVPRHVSPV
TPPREVPIYA NRFPVPTYAA KQPQQFPSRP PPPQPKVSSQ
GNLIPARPAP APPLYSSLT

The Reprolysin (M12B) Family Zinc Metalloprotease Domain of the protein is at approximately residues 212-406); the Disintegrin Domain of the protein is at approximately residues 423-497.

In certain embodiments, ADAM9-binding molecules of the invention (e.g., scFvs, antibodies, bispecific diabodies, etc.) are characterized by any one, two, three, four, five, six, seven, or eight of the following criteria:
(1) the ability to immunospecifically bind human ADAM9 as endogenously expressed on the surface of a cancer cell;
(2) specifically binds human and non-human primate ADAM9 (e.g., ADAM9 of cynomolgus monkey) with a similar binding affinity;
(3) specifically binds human ADAM9 with an equilibrium binding constant (K_{D}) of 4 nM or less;
(4) specifically binds non-human primate ADAM9 with an equilibrium binding constant (K_{D}) of 4 nM or less
(5) specifically binds human ADAM9 with an on rate (ka) of 5×10⁵ M⁻¹min⁻¹ or more;
(6) specifically binds non-human primate ADAM9 with an on rate (ka) of 1×10⁶ M⁻¹min⁻¹ or more;
(7) specifically binds human ADAM9 with an off rate (kd) of 1×10⁻³ min⁻¹ or less;
(8) specifically binds non-human primate ADAM9 with an off rate (kd) of 9×10⁻⁴ min⁻¹ or less;
(9) optimized to have at least 100-fold enhancement (e.g., at least 100-fold, at least 150-fold, at least 200-fold, at least 250-fold, at least 300-fold, at least 350-fold, at least 400-fold, at least 450-fold, at least 500-fold, at least 550-fold, or at least 600-fold enhancement) in binding affinity (e.g., as measured by BIACORE® analysis) to cyno ADAM9 and retains high affinity binding to human ADAM9 (e.g., as measured by BIACORE® analysis) as compared to the chimeric or murine parental antibody.

As described herein, the binding constants of an ADAM9-binding molecule may be determined using surface plasmon resonance e.g., via a BIACORE® analysis. Surface plasmon resonance data may be fitted to a 1:1 Langmuir binding model (simultaneous ka kd) and an equilibrium binding constant K_{D} calculated from the ratio of rate constants kd/ka. Such binding constants may be determined for a monovalent ADAM9-binding molecule (i.e., a molecule comprising a single ADAM9 epitope-binding site), a bivalent ADAM9-binding molecule (i.e., a molecule comprising two ADAM9 epitope-binding sites), or ADAM9-binding molecules having higher valency (e.g., a molecule comprising three, four, or more ADAM9 epitope-binding sites).

The present invention particularly encompasses ADAM9-binding molecules (e.g., antibodies, diabodies, trivalent binding molecules, etc.) comprising anti-ADAM9 Light Chain Variable (VL) Domain(s) and anti-ADAM9 Heavy Chain Variable (VH) Domain(s) that immunospecifically bind to an epitope of a human ADAM9 polypeptide. Unless otherwise stated, all such ADAM9-binding molecules are capable of immunospecifically binding to human ADAM9. As used herein such ADAM9 Variable Domains are referred to as “anti-ADAM9-VL” and “anti-ADAM9-VH,” respectively.

### V. Murine Anti-Human ADAM9 Antibodies

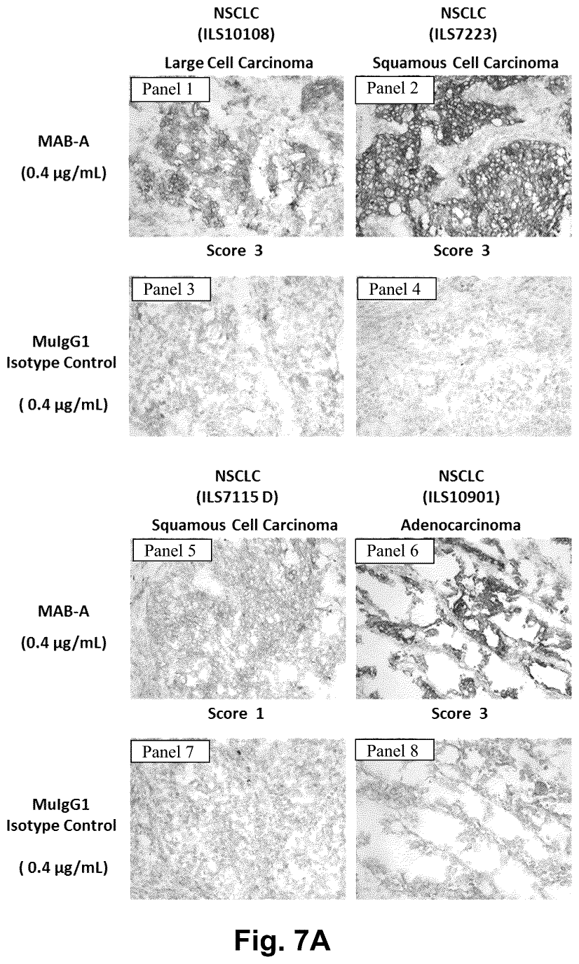

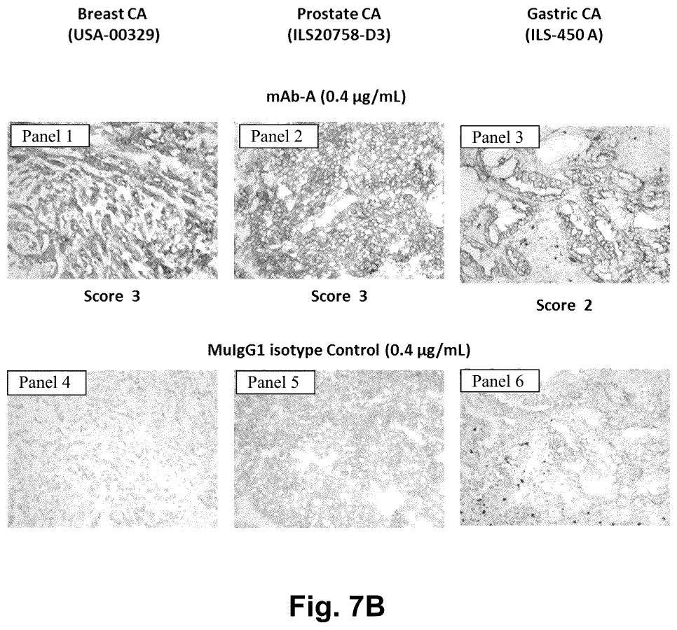

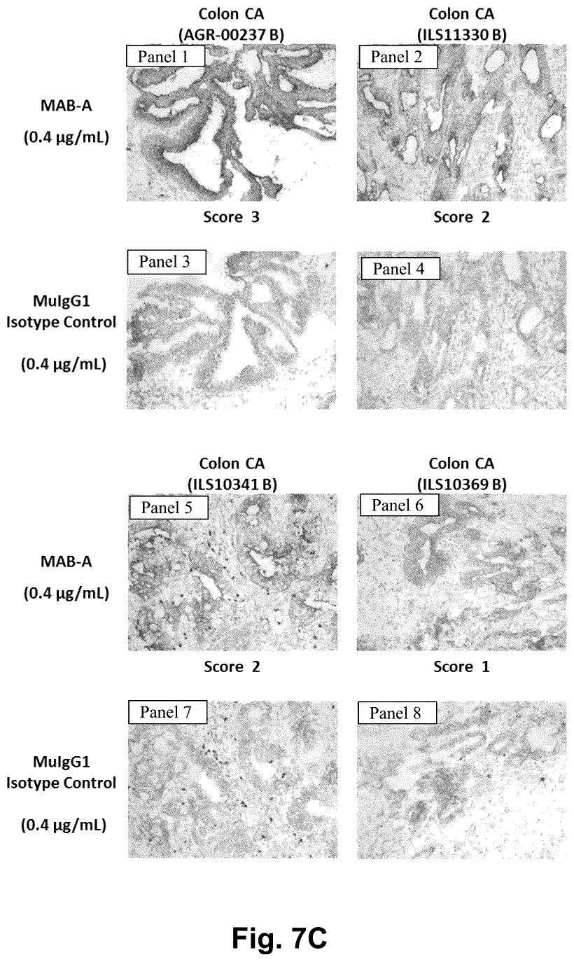

A murine anti-ADAM9 antibody that blocks the target protein processing activity of ADAM9, is internalized and having anti-tumor activity was identified (see, e.g., U.S. Pat. No. 8,361,475). This antibody, designated in U.S. Pat. Nos. 7,674,619 and 8,361,475 as an “anti-KID24” antibody produced by hybridoma clone ATCC PTA-5174, is designated herein as “MAB-A.” MAB-A exhibits strong preferential binding to tumors over normal tissues (see, FIGS. 7A-7C). MAB-A exhibited little or no staining across a large panel of normal cell types (Table 1).

TABLE 1

| Tissue | MAB-A (1.25 μg/mL) |
|---|---|
| Adrenal | Negative |
| Bladder | Negative |
| Bone Marrow | Negative |
| Breast | Negative |
| Cerebellum | Negative |
| Cerebrum | ND |
| Cervix | Negative |
| Colon | Negative |
| Esophagus | Smooth Muscle +/− to 1+ (gr c) <5% |
| Ovaduct | Negative |
| Heart | Negative |
| Kidney | Negative |
| Liver | Negative |
| Lung | Negative |
| Lymph Node | Negative |
| Ovary | Negative |
| Pancreas | Very rare (possible acinar) 1+ (c) |
| Parathyroid | Epithelium parenchymal cells 1+ (gr c), 1% |
| | Cells (favor chief cells) 2+ (m, c)5% 1+ (m, c) 10% |
| | apical primarily |
| Pituitary | Posterior lobe cells (possibly neural cells and/or |
| | pituicytes 1+ (c > m) <5% |
| Placenta | Vascular lining cells within chorionic plate |
| | 1+ (gr c > m) |
| | Mesenchymal cells of chorionic plate 1-2+ (gr c), 5% |
| Prostate | Glandular epithelium 2+ (gr c)5% and 1+ (gr c) 5% |
| Retina + | Favor negative (pigmented epi layer 3-4+ (gr c) due |
| Ciliary Body | to pigment not stained) |
| Submandibular | Ductal epi +/− (c) 10% |
| Gland | |
| Skeletal Muscle | Negative |
| Skin | Negative |
| Small Intestine | Negative |
| Spinal Cord | Neuropil 1+ (gr c) <1% |
| Spleen | Negative |
| Stomach | Negative |
| Testis | Seminiferous tubule 1+ (gr c) <5% |
| | Interstitial cells (possibly Leydig cells) |
| | 2-3+ (gr c) <5% and 1+ (gr c) 10% |
| Thyroid | Negative |
| Tonsil | Endo cells 2-3+ (c, m) <5% and 1+ (m, c) 15% |
| Ureter | Transitional epithelium 1+ (m, c) <5% |
| | and 1+ (m, c) 5%; Endo cells 1+ (c) <5% |
| Uterus | Negative |
| A498 Cell | 2-3+ (m, c), 50%, 1+ (m, c) 45% |
| Pellet | |

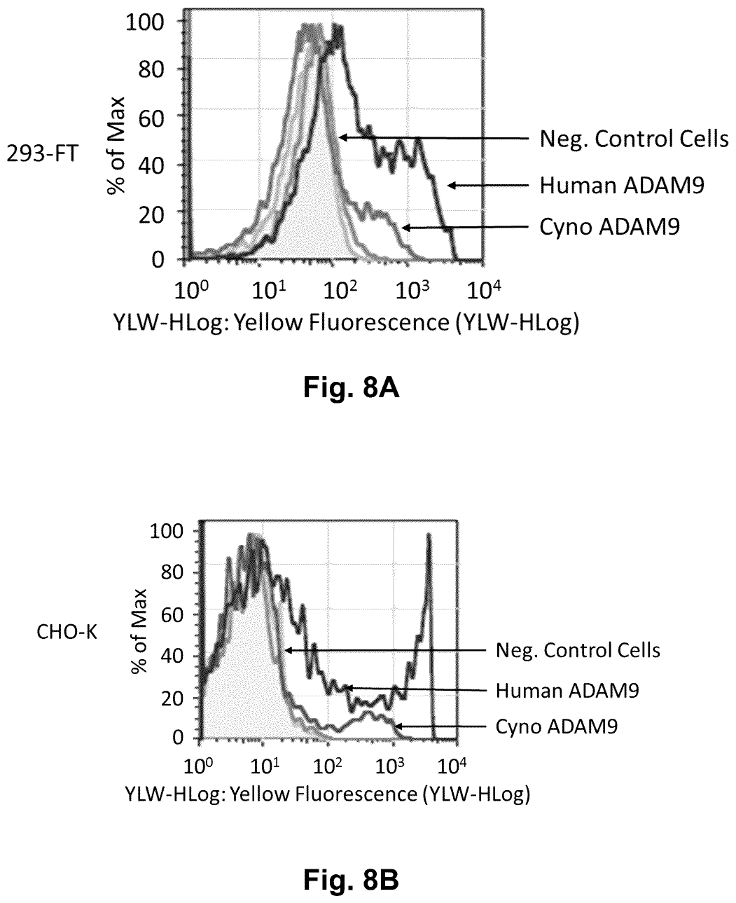

As shown in FIGS. 8A-8B, MAB-A binds human ADAM9 with high affinity, but binds non-human primate (e.g., cynomolgus monkey) ADAM9 to a lesser extent.

The amino acid sequences of the VL and VH Domains of MAB-A are provided below. The VH and VL Domains of MAB-A were humanized and the CDRs optimized to improve affinity and/or to remove potential amino acid liabilities. The CDR_{H}3 was further optimized to enhance binding to non-human primate ADAM9 while maintaining its high affinity for human ADAM9.

The preferred anti-human ADAM9-binding molecules of the present invention possess the 1, 2 or all 3 of the CDR_{H}s of a VH Domain and/or 1, 2 or all 3 of the CDR_{L}s of the VL Domain of an optimized variant of MAB-A, and preferably further possess the humanized framework regions (“FRs”) of the VH and/or VL Domains of humanized MAB-A. Other preferred anti-human ADAM9-binding molecules of the present invention possess the entire VH and/or VL Domains of a humanized/optimized variant of MAB-A. Such preferred anti-human ADAM9-binding molecules include antibodies, bispecific (or multispecific) antibodies, chimeric or humanized antibodies, BITE® molecules, diabodies, etc., as well as such binding molecules that additionally comprise a naturally occurring or a variant Fc Region.

The invention particularly relates to ADAM9-binding molecules comprising an ADAM9 binding domain that possess:
(A) (1) the three CDR_{H}s of the VH Domain of MAB-A; and
   (2) the four FRs of the VH Domain of a humanized variant of MAB-A; or
(B) (1) the three CDR_{L}s of the VL Domain of MAB-A; and
   (2) the four FRs of the VL Domain of a humanized variant of MAB-A; or
(C) the three CDR_{H}s of the VH Domain of an optimized variant of MAB-A; and the three CDR_{L}s of the VL Domain of MAB-A; or
(D) the three CDR_{H}s of the VH Domain of MAB-A; and the three CDR_{L}s of the VL Domain of an optimized variant MAB-A; or
(E) the three CDR_{H}s of the VH Domain of an optimized variant of MAB-A; and the three CDR_{L}s of the VL Domain of an optimized MAB-A; or
(F) (1) the three CDR_{H}s of the VH Domain of an optimized variant of MAB-A; and
   (2) the four FRs of the VH Domain of a humanized variant of MAB-A; or
(G) (1) the three CDR_{L}s of the VL Domain of an optimized variant of MAB-A; and
   (2) the four FRs of the VL Domain of a humanized variant of MAB-A; or
(H) (1) the VH Domain of a humanized/optimized variant of MAB-A; and
   (2) the VL Domain of a humanized/optimized variant of MAB-A. Murine Antibody “MAB-A”

The amino acid sequence of the VH Domain of the murine anti-ADAM9 antibody MAB-A is SEQ ID NO:7 (the CDR_{H} residues are shown underlined):

QVQLQQPGAE LVKPGASVKL SCKASGYTFT SYWMHWVKQR
PGQGLEWIGE IIPINGHTNY NEKFKSKATL TLDKSSSTAY
MQLSSLASED SAVYYCARGG YYYYGSRDYF DYWGQGTTLT VSS

The amino acid sequence of the CDR_{H}1 Domain of MAB-A is (SEQ ID NO:8): SYWMH.

The amino acid sequence of the CDR_{H}2 Domain of MAB-A is (SEQ ID NO:9): EIIPINGHTNYNEKFKS.

The amino acid sequence of the CDR_{H}3 Domain of MAB-A is (SEQ ID NO:10): GGYYYYGSRDYFDY.

The amino acid sequence of the VL Domain of the murine anti-ADAM9 antibody MAB-A is SEQ ID NO:11 (the CDR_{L} residues are shown underlined):

DIVLTQSPAS LAVSLGQRAT ISCKASQSVD YDGDSYMNWY
QQIPGQPPKL LIYAASDLES GIPARFSGSG SGTDFTLNIH
PVEEEDAATY YCQQSHEDPF TFGGGTKLEI K

The amino acid sequence of the CDR_{L}1 Domain of MAB-A is (SEQ ID NO:12): KASQSVDYDGDSYMN.

The amino acid sequence of the CDR_{L}2 Domain of MAB-A is (SEQ ID NO:13): AASDLES.

The amino acid sequence of the CDR_{L}3 Domain of MAB-A is (SEQ ID NO:14): QQSHEDPFT.

### VI. Exemplary Humanized/Optimized Anti-ADAM9-VH and VL Domains

### 1. Variant VH Domains of MAB-A

The amino acid sequences of certain preferred humanized/optimized anti-ADAM9-VH Domains of MAB-A are variants of the ADAM9-VH Domain of MAB-A (SEQ ID NO:7) and are represented by SEQ ID NO:15 (CDR_{H} residues are shown underlined):

EVQLVESGGG LVKPGGSLRL SCAASGFTFS SYWX₁HWVRQA
PGKGLEWVGE IIPIX₂GHTNY NEX₃FX₄X₅RFTI
SLDNSKNTLY LQMGSLRAED TAVYYCARGG
YYYYX₆X₇X₈X₉X₁₀X₁₁ DYWGQGTTVT VSS

wherein: X₁, X₂, X₃, X₄, X₅, and X₆ are independently selected,
wherein: X₁ is M or I; X₂ is N or F;
   X₃ is K or R; X₄ is K or Q;
   X₅ is S or G, and X₆ is P, F, Y, W, I, L, V, T, G or D;
wherein: X₇, X₈, X₉, X₁₀, and X₁₁ are selected such that:

(A) when χ₆ is P:
X₇ is K or R;
X₈ is F or M;
X₉ is G;
X₁₀ is W or F; and
X₁₁ is M, L or K;
(B) when X₆ is F, Y or W:
X₇ is N or H;
X₈ is S or K;
X₉ is G or A;
X₁₀ is T or V; and
X₁₁ is M, L or K;
(C) when X₆ is I, L or V:
X₇ is G;
X₈ is K;
X₉ is G or A;
X₁₀ is V; and
X₁₁ is M, L or K;
(D) when X₆ is T:
X₇ is G;
X₈ is K, M or N;
X₉ is G;
X₁₀ is V or T; and
X₁₁ is L or M;
(E) when X₆ is G:
X₇ is G;
X₈ is S;
X₉ is G;
X₁₀ is V; and
X₁₁ is L;
and (F) when χ₆ is D:
X₇ is S;
X₈ is N;
X₉ is A;
X₁₀ is V; and
X₁₁ is L.

The amino acid sequences of a preferred humanized anti-ADAM9 VH Domain of MAB-A: hMAB-A VH(1) (SEQ ID NO:16) and of the certain preferred humanized/optimized anti-ADAM9-VH Domains of MAB-A:

(SEQ ID NO: 17)

hMAB-A VH(2)

(SEQ ID NO: 18)

hMAB-A VH(3)

(SEQ ID NO: 19)

hMAB-A VH(4)

(SEQ ID NO: 20)

hMAB-A VH(2A)

(SEQ ID NO: 21)

hMAB-A VH(2B)

(SEQ ID NO: 22)

hMAB-A VH(2C)

(SEQ ID NO: 23)

hMAB-A VH(2D)

(SEQ ID NO: 24)

hMAB-A VH(2E)

(SEQ ID NO: 25)

hMAB-A VH(2F)

(SEQ ID NO: 26)

hMAB-A VH(2G)

(SEQ ID NO: 27)

hMAB-A VH(2H)

(SEQ ID NO: 28)

hMAB-A VH(2I)
and

(SEQ ID NO: 29)

hMAB-A VH(2J)

are presented below (CDR_{H} residues are shown in single underline; differences relative to hMAB-A VH(1) (SEQ ID NO:7) are shown in double underline).

hMAB-A VH(1) (SEQ ID NO: 16):
EVQLVESGGG LVKPGGSLRL SCAASGFTFS SYWMHWVRQA PGKGLEWVGE
IIPINGHTNY NEKFKSRFTI SLDNSKNTLY LQMGSLRAED TAVYYCARGG
YYYYGSRDYF DYWGQGTTVT VSS
hMAB-A VH(2) (SEQ ID NO: 17):
EVQLVESGGG LVKPGGSLRL SCAASGFTFS SYWMHWVRQA PGKGLEWVGE [symbol]
NEKFKSRFTI SLDNSKNTLY LQMGSLRAED TAVYYCARGG
YYYYGSRDYF DYWGQGTTVT VSS
hMAB-A VH(3) (SEQ ID NO: 18):
EVQLVESGGG LVKPGGSLRL SCAASGFTFS SYWMHWVRQA PGKGLEWVGE [symbol] [symbol]
SLDNSKNTLY LQMGSLRAED TAVYYCARGG
YYYYGSRDYF DYWGQGTTVT VSS
hMAB-A VH(4) (SEQ ID NO: 19):
EVQLVESGGG LVKPGGSLRL SCAASGFTFS [symbol] PGKGLEWVGE [symbol] [symbol]
SLDNSKNTLY LQMGSLRAED TAVYYCARGG
YYYYGSRDYF DYWGQGTTVT VSS
hMAB-A VH(2A) (SEQ ID NO: 20):
EVQLVESGGG LVKPGGSLRL SCAASGFTFS SYWMHWVRQA PGKGLEWVGE [symbol]
NEKFKSRFTI SLDNSKNTLY LQMGSLRAED TAVYYCARGG [symbol]
DYWGQGTTVT VSS
hMAB-A VH(2B) (SEQ ID NO: 21):
EVQLVESGGG LVKPGGSLRL SCAASGFTFS SYWMHWVRQA PGKGLEWVGE [symbol]
NEKFKSRFTI SLDNSKNTLY LQMGSLRAED TAVYYCARGG [symbol]
DYWGQGTTVT VSS
hMAB-A VH(2C) (SEQ ID NO: 22):
EVQLVESGGG LVKPGGSLRL SCAASGFTFS SYWMHWVRQA PGKGLEWVGE [symbol]
NEKFKSRFTI SLDNSKNTLY LQMGSLRAED TAVYYCARGG [symbol]
DYWGQGTTVT VSS
hMAB-A VH(2D) (SEQ ID NO: 23):
EVQLVESGGG LVKPGGSLRL SCAASGFTFS SYWMHWVRQA PGKGLEWVGE [symbol]
NEKFKSRFTI SLDNSKNTLY LQMGSLRAED TAVYYCARGG [symbol]
DYWGQGTTVT VSS
hMAB-A VH(2E) (SEQ ID NO: 24):
EVQLVESGGG LVKPGGSLRL SCAASGFTFS SYWMHWVRQA PGKGLEWVGE [symbol]
NEKFKSRFTI SLDNSKNTLY LQMGSLRAED TAVYYCARGG [symbol]
DYWGQGTTVT VSS
hMAB-A VH(2F) (SEQ ID NO: 25):
EVQLVESGGG LVKPGGSLRL SCAASGFTFS SYWMHWVRQA PGKGLEWVGE [symbol]
NEKFKSRFTI SLDNSKNTLY LQMGSLRAED TAVYYCARGG [symbol]
DYWGQGTTVT VSS
hMAB-A VH(2G) (SEQ ID NO: 26):
EVQLVESGGG LVKPGGSLRL SCAASGFTFS SYWMHWVRQA PGKGLEWVGE [symbol]
NEKFKSRFTI SLDNSKNTLY LQMGSLRAED TAVYYCARGG [symbol]
DYWGQGTTVT VSS
hMAB-A VH(2H) (SEQ ID NO: 27):
EVQLVESGGG LVKPGGSLRL SCAASGFTFS SYWMHWVRQA PGKGLEWVGE [symbol]
NEKFKSRFTI SLDNSKNTLY LQMGSLRAED TAVYYCARGG [symbol]
DYWGQGTTVT VSS
hMAB-A VH(2I) (SEQ ID NO: 28):
EVQLVESGGG LVKPGGSLRL SCAASGFTFS SYWMHWVRQA PGKGLEWVGE [symbol]
NEKFKSRFTI SLDNSKNTLY LQMGSLRAED TAVYYCARGG [symbol]
DYWGQGTTVT VSS
hMAB-A VH(2J) (SEQ ID NO: 29):
EVQLVESGGG LVKPGGSLRL SCAASGFTFS SYWMHWVRQA PGKGLEWVGE [symbol]
NEKFKSRFTI SLDNSKNTLY LQMGSLRAED TAVYYCARGG [symbol]
DYWGQGTTVT VSS

Suitable human amino acid sequences for the FRs of a humanized and/or optimized anti-ADAM9-VH Domain of MAB-A are:

FR_{H}1 Domain (SEQ ID NO: 30):
EVQLVESGGGLVKPGGSLRLSCAASGFTFS
FR_{H}2 Domain (SEQ ID NO: 31):
WVRQAPGKGLEWVG
FR_{H}3 Domain (SEQ ID NO: 32):
RFTISLDNSKNTLYLQMGSLRAEDTAVYYCAR
FR_{H}4 Domain (SEQ ID NO: 33):
WGQGTTVTVSS

Suitable alternative amino acid sequences for the CDR_{H}1 Domain of an anti-ADAM9-VH Domain of MAB-A include:

SEQ ID NO: 8:
SYWMH
SEQ ID NO: 34:
SYWIH

Suitable alternative amino acid sequences for the CDR_{H}2 Domain of an anti-ADAM9-VH Domain of MAB-A include:

SEQ ID NO: 9:
EIIPINGHTNYNEKFKS
SEQ ID NO: 35:
EIIPIFGHTNYNEKFKS
SEQ ID NO: 36:
EIIPIFGHTNYNERFQG

Suitable alternative amino acid sequences for the CDR_{H}3 Domain of an anti-ADAM9-VH Domain of MAB-A include:

SEQ ID NO: 10:
GGYYYYGSRDYFDY
SEQ ID NO: 37:
GGYYYYFNSGTLDY
SEQ ID NO: 38:
GGYYYYIGKGVLDY
SEQ ID NO: 39:
GGYYYYPRFGWLDY
SEQ ID NO: 40:
GGYYYYTGKGVLDY
SEQ ID NO: 41:
GGYYYYDSNAVLDY
SEQ ID NO: 42:
GGYYYYFHSGTLDY
SEQ ID NO: 43:
GGYYYYFNKAVLDY
SEQ ID NO: 44:
GGYYYYGGSGVLDY
SEQ ID NO: 45:
GGYYYYPRQGFLDY
SEQ ID NO: 46:
GGYYYYYNSGTLDY

Accordingly, the present invention encompasses ADAM9 binding molecules having a VH domain comprising:
(1) a CDR_{H}1 Domain having the amino acid sequence:

SEQ ID NO: 47:
SYWX₁H

wherein: X₁ is M or I;
(2) a CDR_{H}2 Domain having the amino acid sequence:

SEQ ID NO: 48:
EIIPIX₂GHTNYNEX₃FX₄X₅

wherein: X₂, X₃, X₄, and X₅ are independently selected, and
wherein: X₂ is N or F; X₃ is K or R;
   X₄ is K or Q; and X₅ is S or G.
and
(3) a CDR_{H}3 Domain having the amino acid sequence:

SEQ ID NO: 49:
GGYYYYX₆X₇X₈X₉x₁₀X₁₁DY

wherein: X₆, is P, F, Y, W, I, L, V, T, G or D, and X₇, X₈, X₉, X₁₀, and X₁₁ are selected such that:

(A) when X₆ is P:
X₇ is K or R;
X₈ is F or M;
X₉ is G;
X₁₀ is W or F; and
X₁₁ is M, L or K;
(B) when X₆ is F, Y or W:
X₇ is N or H;
X₈ is S or K;
X₉ is G or A;
X₁₀ is T or V; and
X₁₁ is M, L or K;
(C) when X₆ is I, L or V:
X₇ is G;
X₈ is K;
X₉ is G or A;
X₁₀ is V; and
X₁₁ is M, L or K;
(D) when X₆ is T:
X₇ is G;
X₈ is K, M or N;
X₉ is G;
X₁₀ is V or T; and
X₁₁ is L or M;
(E) when X₆ is G:
X₇ is G;
X₈ is S;
X₉ is G;
X₁₀ is V; and
X₁₁ is L;
and (F) when X₆ is D:
X₇ is S;
X₈ is N;
X₉ is A;
X₁₀ is V; and
X₁₁ is L.

A first exemplary humanized/optimized IgG1 Heavy Chain of a derivative/variant of MAB-A contains the hMAB-A VH (2) Domain (SEQ ID NO:17), and has the amino acid sequence (SEQ ID NO:50):

EVQLVESGGG LVKPGGSLRL SCAASGFTFS SYWMHWVRQA
PGKGLEWVGE IIPIFGHTNY NEKFKSRFTI SLDNSKNTLY
LQMGSLRAED TAVYYCARGG YYYYGSRDYF DYWGQGTTVT
VSSASTKGPS VFPLAPSSKS TSGGTAALGC LVKDYFPEPV
TVSWNSGALT SGVHTFPAVL QSSGLYSLSS VVTVPSSSLG
TQTYICNVNH KPSNTKVDKR VEPKSCDKTH TCPPCPAPEL
LGGPSVFLFP PKPKDTLMIS RTPEVTCVVV DVSHEDPEVK
FNWYVDGVEV HNAKTKPREE QYNSTYRVVS VLTVLHQDWL
NGKEYKCKVS NKALPAPIEK TISKAKGQPR EPQVYTLPPS
REEMTKNQVS LTCLVKGFYP SDIAVEWESN GQPENNYKTT
PPVLDSDGSF FLYSKLTVDK SRWQQGNVFS CSVMHEALHN
HYTQKSLSLS PGX

wherein X is a lysine (K) or is absent.

A second exemplary humanized/optimized IgG1 Heavy Chain of a derivative/variant of MAB-A contains the hMAB-A VH (2C) Domain (SEQ ID NO:22), and has the amino acid sequence (SEQ ID NO:51):

EVQLVESGGG LVKPGGSLRL SCAASGFTFS SYWMHWVRQA
PGKGLEWVGE IIPIFGHTNY NEKFKSRFTI SLDNSKNTLY
LQMGSLRAED TAVYYCARGG YYYYPRFGWL DYWGQGTTVT
VSSASTKGPS VFPLAPSSKS TSGGTAALGC LVKDYFPEPV
TVSWNSGALT SGVHTFPAVL QSSGLYSLSS VVTVPSSSLG
TQTYICNVNH KPSNTKVDKR VEPKSCDKTH TCPPCPAPEL
LGGPSVFLFP PKPKDTLMIS RTPEVTCVVV DVSHEDPEVK
FNWYVDGVEV HNAKTKPREE QYNSTYRVVS VLTVLHQDWL
NGKEYKCKVS NKALPAPIEK TISKAKGQPR EPQVYTLPPS
REEMTKNQVS LTCLVKGFYP SDIAVEWESN GQPENNYKTT
PPVLDSDGSF FLYSKLTVDK SRWQQGNVFS CSVMHEALHN
HYTQKSLSLS PGX

wherein X is a lysine (K) or is absent.

A third exemplary humanized/optimized IgG1 Heavy Chain of a derivative/variant of MAB-A contains the hMAB-A VH (2I) Domain (SEQ ID NO:28), and has the amino acid sequence (SEQ ID NO:52):

EVQLVESGGG LVKPGGSLRL SCAASGFTFS SYWMHWVRQA
PGKGLEWVGE IIPIFGHTNY NEKFKSRFTI SLDNSKNTLY
LQMGSLRAED TAVYYCARGG YYYYPRQGFL DYWGQGTTVT
VSSASTKGPS VFPLAPSSKS TSGGTAALGC LVKDYFPEPV
TVSWNSGALT SGVHTFPAVL QSSGLYSLSS VVTVPSSSLG
TQTYICNVNH KPSNTKVDKR VEPKSCDKTH TCPPCPAPEL
LGGPSVFLFP PKPKDTLMIS RTPEVTCVVV DVSHEDPEVK
FNWYVDGVEV HNAKTKPREE QYNSTYRVVS VLTVLHQDWL
NGKEYKCKVS NKALPAPIEK TISKAKGQPR EPQVYTLPPS
REEMTKNQVS LTCLVKGFYP SDIAVEWESN GQPENNYKTT
PPVLDSDGSF FLYSKLTVDK SRWQQGNVFS CSVMHEALHN
HYTQKSLSLS PGX

wherein X is a lysine (K) or is absent.

As provided herein, the CH2-CH3 Domains of the Fc Region may be engineered for example, to reduce effector function. In certain embodiments, the CH2-CH3 Domains of the exemplary humanized/optimized IgG1 Heavy Chains of the invention comprise one or more substitutions selected from: L234A and L235A.

Thus, a fourth exemplary humanized/optimized IgG1 Heavy Chain of a derivative/variant of MAB-A contains the hMAB-A VH (2I) Domain (SEQ ID NO:28), and further comprises the substitutions L234A, and L235A in the CH2-CH3 Domains of the Fc Region (SEQ ID NO:106), underlined below) and has the amino acid sequence (SEQ ID NO:202):

EVQLVESGGG LVKPGGSLRL SCAASGFTFS SYWMHWVRQA
PGKGLEWVGE IIPIFGHTNY NEKFKSRFTI SLDNSKNTLY
LQMGSLRAED TAVYYCARGG YYYYPRQGFL DYWGQGTTVT
VSSASTKGPS VFPLAPSSKS TSGGTAALGC LVKDYFPEPV
TVSWNSGALT SGVHTFPAVL QSSGLYSLSS VVTVPSSSLG
TQTYICNVNH KPSNTKVDKR VEPKSCDKTH TCPPCPAPEA
AGGPSVFLFP PKPKDTLMIS RTPEVTCVVV DVSHEDPEVK
FNWYVDGVEV HNAKTKPREE QYNSTYRVVS VLTVLHQDWL
NGKEYKCKVS NKALPAPIEK TISKAKGQPR EPQVYTLPPS
REEMTKNQVS LTCLVKGFYP SDIAVEWESN GQPENNYKTT
PPVLDSDGSF FLYSKLTVDK SRWQQGNVFS CSVMHEALHN
HYTQKSLSLS PGX

wherein X is a lysine (K) or is absent.

### 2. Variant VL Domains of MAB-A

The amino acid sequences of preferred humanized/optimized anti-ADAM9-VL Domains of MAB-A are variants of the ADAM9-VL Domain of MAB-A (SEQ ID NO:11) and are represented by SEQ ID NO:53 (CDR_{L} residues are shown underlined):

DIVMTQSPDS LAVSLGERAT ISCX₁₂ASQSVD YX₁₃GDSYX₁₄NWY
QQKPGQPPKL LIYAASDLES GIPARFSGSG SGTDFTLTIS
SLEPEDFATY YCQQSX₁₅X₁₆X₁₇PF TFGQGTKLEI K

wherein: X₁₂, X₁₃, X₁₄, X₁₅, X₁₆, and X₁₇, are independently selected, and
wherein: X₁₂ is K or R; X₁₃ is D or S;
X₁₄ is M or L; X₁₅ is H or Y;
X₁₆ is E or S; and X₁₇ is D or T.

The amino acid sequences of a preferred humanized anti-ADAM9-VL Domain of MAB-A: hMAB-A VL(1) (SEQ ID NO:54), and of certain preferred humanized/optimized anti-ADAM9-VL Domains of MAB-A: hMAB-A VL(2) (SEQ ID NO:55), hMAB-A VL(3) (SEQ ID NO:56), and hMAB-A VL(4) (SEQ ID NO:57), are presented below (CDR_{L} residues are shown in single underline; differences relative to hMAB-A VL(1) (SEQ ID NO:54) are shown in double underline).

hMAB-A VL(1) (SEQ ID NO: 54):
DIVMTQSPDS LAVSLGERAT ISCKASQSVD YDGDSYMNWY
QQKPGQPPKL LIYAASDLES GIPARFSGSG SGTDFTLTIS
SLEPEDFATY YCQQSHEDPF TFGQGTKLEI K
hMAB-A VL(2) (SEQ ID NO: 55):
DIVMTQSPDS LAVSLGERAT ISCKASQSVD YSGDSYMNWY
QQKPGQPPKL LIYAASDLES GIPARFSGSG SGTDFTLTIS
SLEPEDFATY YCQQSHEDPF TFGQGTKLEI K
hMAB-A VL(3) (SEQ ID NO: 56):
DIVMTQSPDS LAVSLGERAT ISCRASQSVD YSGDSYMNWY
QQKPGQPPKL LIYAASDLES GIPARFSGSG SGTDFTLTIS
SLEPEDFATY YCQQSHEDPF TFGQGTKLEI K
hMAB-A VL(4) (SEQ ID NO: 57):
DIVMTQSPDS LAVSLGERAT ISCRASQSVD YSGDSYLNWY
QQKPGQPPKL LIYAASDLES GIPARFSGSG SGTDFTLTIS
SLEPEDFATY YCQQSYSTPF TFGQGTKLEI K

Accordingly, suitable human amino acid sequences for the FRs of a humanized and/or optimized anti-ADAM9-VL Domain of MAB-A are:

FR_{L}1 Domain (SEQ ID NO: 58):
DIVMTQSPDSLAVSLGERATISC
FR_{L}2 Domain (SEQ ID NO: 59):
WYQQKPGQPPKLLIY
FR_{L}3 Domain (SEQ ID NO: 60):
GIPARFSGSGSGTDFTLTISSLEPEDFATYYC
FR_{L}4 Domain (SEQ ID NO: 61):
FGQGTKLEIK

Suitable alternative amino acid sequences for the CDR_{L}1 Domain of an anti-ADAM9-VL Domain include:

SEQ ID NO: 12: KASQSVDYDGDSYMN
SEQ ID NO: 62: KASQSVDYSGDSYMN
SEQ ID NO: 63: RASQSVDYSGDSYMN
SEQ ID NO: 64: RASQSVDYSGDSYLN

Suitable alternative amino acid sequences for the CDR_{L}3 Domain of an anti-ADAM9-VL Domain include:

SEQ ID NO: 14: QQSHEDPFT
SEQ ID NO: 65: QQSYSTPFT

Accordingly, the present invention encompasses anti-ADAM9 antibody VL Domain comprising:
(1) a CDR_{L}1 Domain having the amino acid sequence:

SEQ ID NO: 66: X₁₂ASQSVDYX₁₃GDSYX₁₄N

wherein: X₁₂, X₁₃, X₁₄, are independently selected, and
wherein: X₁₂ is K or R; X₁₃ is D or S; and X₁₄ is M or L;
(2) a CDR_{L}2 Domain having the amino acid sequence:

SEQ ID NO: 13: AASDLES

and
(3) a CDR_{L}3 Domain having the amino acid sequence:

SEQ ID NO: 67: QQSX₁₅X₁₆X₁₇PFT

wherein: X₁₅, X₁₆, and X₁₇, are independently selected, and
wherein: X₁₅ is H or Y; X₁₆ is E or S; and X₁₇ is D or T.

An exemplary humanized/optimized IgG1 Light Chain of a derivative/variant of MAB-A contains the hMAB-A VL (2) Domain (SEQ ID NO:55), and has the amino acid sequence (SEQ ID NO:68):

DIVMTQSPDS LAVSLGERAT ISCKASQSVD YSGDSYMNWY
QQKPGQPPKL LIYAASDLES GIPARFSGSG SGTDFTLTIS
SLEPEDFATY YCQQSHEDPF TFGQGTKLEI KRTVAAPSVF
IFPPSDEQLK SGTASVVCLL NNFYPREAKV QWKVDNALQS
GNSQESVTEQ DSKDSTYSLS STLTLSKADY EKHKVYACEV
THQGLSSPVT KSFNRGEC

Thus, the present invention additionally expressly contemplates ADAM9-binding molecules (e.g., antibodies, diabodies, trivalent binding molecules, etc.) that immunospecifically bind to an epitope of a human ADAM9 polypeptide, and that comprise any of the above-provided MAB-A CDR_{H}1, CDR_{H}2, CDR_{H}3, CDR_{L}1, CDR_{L}2, or CDR_{L}3, and particularly contemplates such ADAM9-binding molecules that comprise one of the above-provided MAB-A CDR_{H}1, one of the above-provided MAB-A CDR_{H}2, one of the above-provided MAB-A CDR_{H}3, one of the above-provided MAB-A CDR_{L}1, one of the above-provided MAB-A CDR_{L}2, and one of the above-provided MAB-A CDR_{L}3.

The invention further contemplates such ADAM9-binding molecules that further comprise any of the above-provided humanized MAB-A FR_{H}1, FR_{H}2, FR_{H}3, or FR_{H}4, FR_{L}1, FR_{L}2, FR_{L}3, or FR_{L}4, and particularly contemplates such ADAM9-binding molecules that comprise FR_{H}1, FR_{H}2, FR_{H}3, and FR_{H}4, and/or that comprise FR_{L}1, FR_{L}2, FR_{L}3, FR_{L}4 and FR_{H}1.

In some embodiments, the ADAM9-binding molecules include a CDR_{H}1 domain, a CDR_{H}2 domain, and a CDR_{H}3 domain and a CDR_{L}1 domain, a CDR_{L}2 domain, and a CDR_{L}3 domain having the sequences selected from the group consisting of:
(a) SEQ ID NOs:8, 35 and 10 and SEQ ID NOs:62, 13, and 14, respectively;
(b) SEQ ID NOs:8, 35 and 10 and SEQ ID NOs:63, 13, and 14, respectively;
(c) SEQ ID NOs:8, 36 and 10 and SEQ ID NOs:63, 13 and 14, respectively;
(d) SEQ ID NOs:34, 36 and 10 and SEQ ID NO:64, 13 and 65, respectively
(e) SEQ ID NOs:8, 35 and 37 and SEQ ID NOs:62, 13 and 14, respectively;
(f) SEQ ID NOs:8, 35 and 38 and SEQ ID NOs:62, 13 and 14, respectively;
(g) SEQ ID NOs:8, 35 and 39 and SEQ ID NOs:62, 13 and 14, respectively;
(h) SEQ ID NOs:8, 35 and 40 and SEQ ID NOs:62, 13 and 14, respectively;
(i) SEQ ID NOs:8, 35 and 41 and SEQ ID NOs:62, 13 and 14, respectively;
(j) SEQ ID NOs:8, 35 and 42 and SEQ ID NOs:62, 13 and 14, respectively;
(k) SEQ ID NOs:8, 35 and 43 and SEQ ID NOs:62, 13 and 14, respectively;
(l) SEQ ID NOs:8, 35 and 44 and SEQ ID NOs:62, 13 and 14, respectively;
(m) SEQ ID NOs:8, 35 and 45 and SEQ ID NOs:62, 13 and 14, respectively; and
(n) SEQ ID NOs:8, 35 and 46 and SEQ ID NOs:62, 13 and 14, respectively.

In particular embodiments, the ADAM9-binding molecules include a CDR_{H}1 domain, a CDR_{H}2 domain, and a CDR_{H}3 domain and a CDR_{L}1 domain, a CDR_{L}2 domain, and a CDR_{L}3 domain having the sequences of SEQ ID NOs:8, 35 and 45 and SEQ ID NOs:62, 13 and 14, respectively.

In some embodiments, the ADAM9-binding molecules of the invention include a heavy chain variable domain (VH) and a light chain variable domain (VL) having sequences that are at least 90%, at least 95%, at least 99%, or are 100% identical to the sequences as follows:
SEQ ID NO:17 and SEQ ID NO:55, respectively;
SEQ ID NO:17 and SEQ ID NO:56, respectively;
SEQ ID NO:18 and SEQ ID NO:56, respectively;
SEQ ID NO:19 and SEQ ID NO:57, respectively;
SEQ ID NO:20 and SEQ ID NO:55, respectively;
SEQ ID NO:21 and SEQ ID NO:55, respectively;
SEQ ID NO:22 and SEQ ID NO:55, respectively;
SEQ ID NO:23 and SEQ ID NO:55, respectively;
SEQ ID NO:24 and SEQ ID NO:55, respectively;
SEQ ID NO:25 and SEQ ID NO:55, respectively;
SEQ ID NO:26 and SEQ ID NO:55, respectively;
SEQ ID NO:27 and SEQ ID NO:55, respectively;
SEQ ID NO:28 and SEQ ID NO:55, respectively; and
SEQ ID NO:29 and SEQ ID NO:55, respectively.

By “substantially identical” or “identical” is meant a polypeptide exhibiting at least 50% identity to a reference amino acid sequence (for example, any one of the amino acid sequences described herein). Preferably, such a sequence is at least 60%, more preferably at least 80% or at least 85%, and more preferably at least 90%, at least 95% at least 99%, or even 100% identical at the amino acid level to the polypeptide sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

In particular embodiments, the ADAM9-binding molecules of the invention include a heavy chain variable domain (VH) and a light chain variable domain (VL) having sequences that are at least 90%, at least 95%, at least 99%, or are 100% identical to the sequences of SEQ ID NO:28 and SEQ ID NO:55, respectively.

In certain embodiments, the ADAM9-binding molecules of the invention comprise a heavy chain and a light chain sequence as follows:
SEQ ID NO:50 and SEQ ID NO:68, respectively;
SEQ ID NO:51 and SEQ ID NO:68, respectively;
SEQ ID NO:52 and SEQ ID NO:68, respectively; and
SEQ ID NO:202 and SEQ ID NO:68, respectively.

The present invention also expressly contemplates ADAM9-binding molecules (e.g., antibodies, diabodies, trivalent binding molecules, etc.) that immunospecifically bind to an epitope of a human ADAM9 polypeptide, and that comprise any of the above-provided humanized/optimized anti-ADAM9 MAB-A VL or VH Domains. The present invention particularly contemplates such ADAM9-binding molecules that comprise any of the following combinations of humanized anti-ADAM9 VL or VH Domains:

hMAB-A VH/hMAB-A VL Combinations

hMAB-A VH(1)/hMAB-A VL(1)
hMAB-A VH(1)/hMAB-A VL(2)
hMAB-A VH(1)/hMAB-A VL(3)
hMAB-A VH(1)/hMAB-A VL(4)
hMAB-A VH(2)/hMAB-A VL(1)
hMAB-A VH(2)/hMAB-A VL(2)
hMAB-A VH(2)/hMAB-A VL(3)
hMAB-A VH(2)/hMAB-A VL(4)
hMAB-A VH(3)/hMAB-A VL(1)
hMAB-A VH(3)/hMAB-A VL(2)
hMAB-A VH(3)/hMAB-A VL(3)
hMAB-A VH(3)/hMAB-A VL(4)
hMAB-A VH(4)/hMAB-A VL(1)
hMAB-A VH(4)/hMAB-A VL(2)
hMAB-A VH(4)/hMAB-A VL(3)
hMAB-A VH(4)/hMAB-A VL(4)
hMAB-A VH(2A)/hMAB-A VL(1)
hMAB-A VH(2A)/hMAB-A VL(2)
hMAB-A VH(2A)/hMAB-A VL(3)
hMAB-A VH(2A)/hMAB-A VL(4)
hMAB-A VH(2B)/hMAB-A VL(1)
hMAB-A VH(2B)/hMAB-A VL(2)
hMAB-A VH(2B)/hMAB-A VL(3)
hMAB-A VH(2B)/hMAB-A VL(4)
hMAB-A VH(2C)/hMAB-A VL(1)
hMAB-A VH(2C)/hMAB-A VL(2)
hMAB-A VH(2C)/hMAB-A VL(3)
hMAB-A VH(2C)/hMAB-A VL(4)
hMAB-A VH(2D)/hMAB-A VL(1)
hMAB-A VH(2D)/hMAB-A VL(2)
hMAB-A VH(2D)/hMAB-A VL(3)
hMAB-A VH(2D)/hMAB-A VL(4)
hMAB-A VH(2E)/hMAB-A VL(1)
hMAB-A VH(2E)/hMAB-A VL(2)
hMAB-A VH(2E)/hMAB-A VL(3)
hMAB-A VH(2E)/hMAB-A VL(4)
hMAB-A VH(2F)/hMAB-A VL(1)
hMAB-A VH(2F)/hMAB-A VL(2)
hMAB-A VH(2F)/hMAB-A VL(3)
hMAB-A VH(2F)/hMAB-A VL(4)
hMAB-A VH(2G)/hMAB-A VL(1)
hMAB-A VH(2G)/hMAB-A VL(2)
hMAB-A VH(2G)/hMAB-A VL(3)
hMAB-A VH(2G)/hMAB-A VL(4)
hMAB-A VH(2H)/hMAB-A VL(1)
hMAB-A VH(2H)/hMAB-A VL(2)
hMAB-A VH(2H)/hMAB-A VL(3)
hMAB-A VH(2H)/hMAB-A VL(4)
hMAB-A VH(2I)/hMAB-A VL(1)
hMAB-A VH(2I)/hMAB-A VL(2)
hMAB-A VH(2I)/hMAB-A VL(3)
hMAB-A VH(2I)/hMAB-A VL(4)
hMAB-A VH(2J)/hMAB-A VL(1)
hMAB-A VH(2J)/hMAB-A VL(2)
hMAB-A VH(2J)/hMAB-A VL(3)
hMAB-A VH(2J)/hMAB-A VL(4)

The present invention specifically encompasses ADAM9-binding molecules comprising (i) a humanized/optimized anti-ADAM9-VL and/or VH Domain as provided above, and (ii) an Fc Region. In particular embodiments, the ADAM9-binding molecules of the present invention are monoclonal antibodies comprising (i) a humanized/optimized anti-ADAM9-VL and/or VH Domain as provided above, and (ii) an Fc Region. In other embodiments, the ADAM9-binding molecules of the present invention are selected from the group consisting of: monoclonal antibodies, multispecific antibodies, synthetic antibodies, chimeric antibodies, single-chain Fvs (scFv), single-chain antibodies, Fab fragments, F(ab′) fragments, disulfide-linked bispecific Fvs (sdFv), BITE® molecules, diabodies, and trivalent binding molecules.

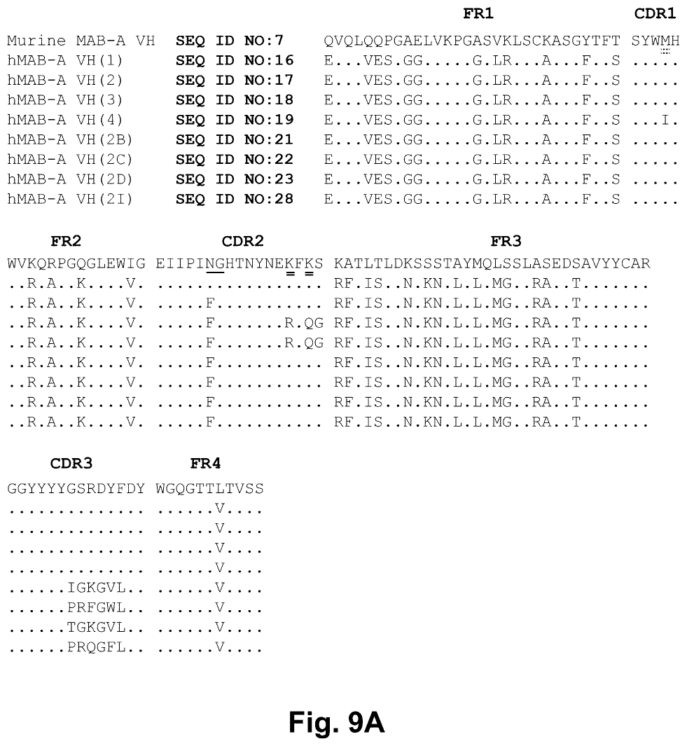

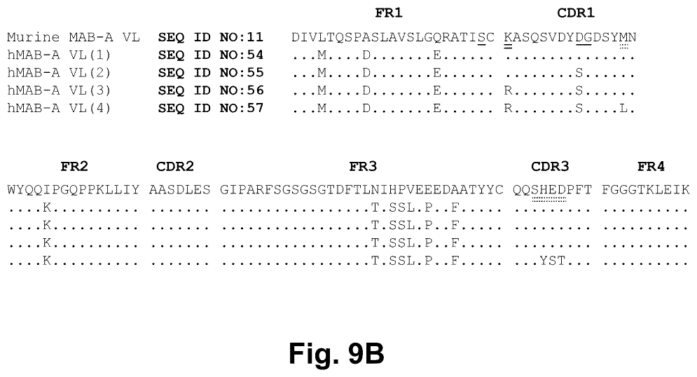

Although particular modifications to anti-ADAM9 VH and VL Domains are summarized above and compared in FIGS. 9A-9B, it is not necessary to modify all or most of these residues when engineering a humanized and/or optimized anti-ADAM9-VH or VL Domain of the invention. The present invention also encompasses minor variations of these VH and VL sequences including, for example, amino acid substitutions of the C-terminal and/or N-terminal amino acid residues which may be introduced to facilitate subcloning.

### VII. Chimeric Antigen Receptors

The ADAM9-binding molecules of the present invention may be monospecific single-chain molecules, such as anti-ADAM9 single-chain variable fragments (“anti-ADAM9-scFvs”) or anti-ADAM9 Chimeric Antigen Receptors (“anti-ADAM9-CARs”). As discussed above, scFvs are made by linking Light and Heavy Chain Variable Domains together via a short linking peptide. First-generation Chimeric Antigen Receptors (“CARs”) typically comprise the intracellular domain from the CD3ζ-chain, which is the primary transmitter of signals from endogenous T-cell Receptors (“TCRs”). Second-generation CARs possess additional intracellular signaling domains from various costimulatory protein receptors (e.g., CD28, 41BB, ICOS, etc.) fused to the cytoplasmic tail of the CAR in order to provide additional signals to the T-cell. Third-generation CARs combine multiple signaling domains, such as CD3ζ-CD28-41BB or CD3ζ-CD28-OX40, in order to further augment their potency (Tettamanti, S. et al. (2013) “Targeting Of Acute Myeloid Leukaemia By Cytokine-Induced Killer Cells Redirected With A Novel CD123-Specific Chimeric Antigen Receptor,” Br. J. Haematol. 161:389-401; Gill, S. et al. (2014) “Efficacy Against Human Acute Myeloid Leukemia And Myeloablation Of Normal Hematopoiesis In A Mouse Model Using Chimeric Antigen Receptor-Modified T Cells,” Blood 123(15): 2343-2354; Mardiros, A. et al. (2013) “T Cells Expressing CD123-Specific Chimeric Antigen Receptors Exhibit Specific Cytolytic Effector Functions And Antitumor Effects Against Human Acute Myeloid Leukemia,” Blood 122:3138-3148; Pizzitola, I. et al. (2014) “Chimeric Antigen Receptors Against CD33/CD123 Antigens Efficiently Target Primary Acute Myeloid Leukemia Cells in vivo,” Leukemia doi:10.1038/leu.2014.62). Chimeric Antigen receptors are discussed in U.S. Pat. Nos. 9,447,194; 9,266,960; 9,212,229; 9,074,000; 8,822,196; and 8,465,743, and in US Patent Publication Nos. 2016/0272718, 2016/0144026, 2016/0130357, 2016/0081314, 2016/0075784, 2016/0058857, 2016/0046729, 2016/0046700, 2016/0045551, 2016/0015750, 2015/0320799, 2015/0307623, 2015/0307564, 2015/0038684, 2014/0134142 and 2013/0280285.

The anti-ADAM9-CARs of the present invention comprise an anti-ADAM9-scFv fused to an intracellular domain of a receptor. The Light Chain Variable (VL) Domain and the Heavy Chain Variable (VH) Domain of the anti-ADAM9-scFv are selected from any of the humanized anti-ADAM9-VL and anti-ADAM9-VH Domains disclosed herein. Preferably, the VH Domain is selected from the group consisting of: hMAB-A VH(1) (SEQ ID NO:16), hMAB-A VH(2) (SEQ ID NO:17), hMAB-A VH(3) (SEQ ID NO:18), hMAB-A VH(4) (SEQ ID NO:19), hMAB-A VH(2A) (SEQ ID NO:20), hMAB-A VH(2B) (SEQ ID NO:21), hMAB-A VH(2C) (SEQ ID NO:22), hMAB-A VH(2D) (SEQ ID NO:23), hMAB-A VH(2E) (SEQ ID NO:24), hMAB-A VH(2F) (SEQ ID NO:25), hMAB-A VH(2G) (SEQ ID NO:26), hMAB-A VH(2H) (SEQ ID NO:27), hMAB-A VH(2I) (SEQ ID NO:28), and hMAB-A VH(2J) (SEQ ID NO:29), and the VL Domain is selected from the group consisting of: hMAB-A VL(1) (SEQ ID NO:54), hMAB-A VL(2) (SEQ ID NO:55), hMAB-A VL(3) (SEQ ID NO:56), and hMAB-A VL(4) (SEQ ID NO:57). Combinations of humanized/optimized anti-ADAM9-VL and anti-ADAM9-VH Domains and combinations of CDR_{H}s and CDR_{L}s that may be used to form such Chimeric Antigen Receptors are presented above.

The intracellular domain of the anti-ADAM9-CARs of the present invention is preferably selected from the intracellular domain of any of: 41BB-CD3ζ, b2c-CD3ζ, CD28, CD28-4-1BB-CD3ζ, CD28-CD3ζ, CD28-FcεRIγ, CD28mut-CD3ζ, CD28-OX40-CD3ζ, CD28-OX40-CD3ζ, CD3ζ, CD4-CD3ζ, CD4-FcεRIγ, CD8-CD3ζ, FcεRIγ, FcεRIγCAIX, Heregulin-CD3ζ, IL-13-CD3ζ, or Ly49H-CD3ζ (Tettamanti, S. et al. (2013) “Targeting Of Acute Myeloid Leukaemia By Cytokine-Induced Killer Cells Redirected With A Novel CD123-Specific Chimeric Antigen Receptor,” Br. J. Haematol. 161:389-401; Gill, S. et al. (2014) “Efficacy Against Human Acute Myeloid Leukemia And Myeloablation Of Normal Hematopoiesis In A Mouse Model Using Chimeric Antigen Receptor-Modified T Cells,” Blood 123(15): 2343-2354; Mardiros, A. et al. (2013) “T Cells Expressing CD123-Specific Chimeric Antigen Receptors Exhibit Specific Cytolytic Effector Functions And Antitumor Effects Against Human Acute Myeloid Leukemia,” Blood 122:3138-3148; Pizzitola, I. et al. (2014) “Chimeric Antigen Receptors Against CD33/CD123 Antigens Efficiently Target Primary Acute Myeloid Leukemia Cells in vivo,” Leukemia doi:10.1038/leu.2014.62).

### VIII. Multispecific ADAM9-Binding Molecules

The present invention is also directed to multispecific (e.g., bispecific, trispecific, etc.) ADAM9-binding molecules comprising an epitope-binding site (preferably comprising 1, 2 or all 3 of the CDR_{H}s of an anti-ADAM9-VH Domain of the invention and/or 1, 2 or all 3 of the CDR_{L}s of an anti-ADAM9-VL Domain of the invention, or such anti-ADAM9-VH Domain and/or such anti-ADAM9-VL Domain) and further comprising a second epitope-binding site that immunospecifically binds to a second epitope, where such second epitope is (i) a different epitope of ADAM9, or (ii) an epitope of a molecule that is not ADAM9. Such multispecific ADAM9-binding molecules preferably comprise a combination of epitope-binding sites that recognize a set of antigens unique to target cells or tissue type. In particular, the present invention relates to multispecific ADAM9-binding molecules that are capable of binding to an epitope of ADAM9 and an epitope of a molecule present on the surface of an effector cell, especially a T lymphocyte, a natural killer (NK) cell or other mononuclear cell. For example, such ADAM9-binding molecules of the present invention may be constructed to comprise an epitope-binding site that immunospecifically binds CD2, CD3, CD8, CD16, T-Cell Receptor (TCR), or NKG2D.

One embodiment of the present invention relates to bispecific ADAM9-binding molecules that are capable of binding to a “first epitope” and a “second epitope,” such epitopes not being identical to one another. Such bispecific molecules comprise “VL1”/“VH1” domains that are capable of binding to the first epitope, and “VL2”/“VH2” domains that are capable of binding to the second epitope. The notation “VL1” and “VH1” denote respectively, the Light Chain Variable Domain and Heavy Chain Variable Domain that bind the “first” epitope of such bispecific molecules. Similarly, the notation “VL2” and “VH2” denote respectively, the Light Chain Variable Domain and Heavy Chain Variable Domain that bind the “second” epitope of such bispecific molecules. It is irrelevant whether a particular epitope is designated as the first vs. the second epitope; such notation having relevance only with respect to the presence and orientation of domains of the polypeptide chains of the binding molecules of the present invention. In one embodiment, one of such epitopes is an epitope of human ADAM9 and the other is a different epitope of ADAM9, or is an epitope of a molecule that is not ADAM9. In particular embodiments, one of such epitopes is an epitope of human ADAM9 and the other is an epitope of a molecule (e.g., CD2, CD3, CD8, CD16, T-Cell Receptor (TCR), NKG2D, etc.) present on the surface of an effector cell, such as a T lymphocyte, a natural killer (NK) cell or other mononuclear cell. In certain embodiments, a bispecific molecule comprises more than two epitope-binding sites. Such bispecific molecules will bind at least one epitope of ADAM9 and at least one epitope of a molecule that is not ADAM9, and may further bind additional epitopes of ADAM9 and/or additional epitopes of a molecule that is not ADAM9.

The present invention particularly relates to bispecific, trispecific and multispecific ADAM9-binding molecules (e.g., bispecific antibodies, bispecific diabodies, trivalent binding molecules, etc.) that possess epitope-binding fragments of antibodies (e.g., VL and VH Domains) that enable them to be able to coordinately bind to at least one epitope of ADAM9 and at least one epitope of a second molecule that is not ADAM9. Selection of the VL and VH Domains of the polypeptide domains of such molecules is coordinated so that the polypeptides chains that make up such multispecific ADAM9-binding molecules assemble to form at least one functional epitope-binding site that is specific for at least one epitope of ADAM9 and at least one functional epitope-binding site that is specific for at least one epitope of a molecule that is not ADAM9. Preferably, the multispecific ADAM9-binding molecules comprise 1, 2 or all 3 of the CDR_{H}s of an anti-ADAM9-VH Domain of the invention and/or 1, 2 or all 3 of the CDR_{L}s of an anti-ADAM9-VL Domain of the invention, or such anti-ADAM9-VH Domain and/or such anti-ADAM9-VL Domain, as provided herein.

### A. Bispecific Antibodies

The present invention encompasses bispecific antibodies capable of simultaneously binding to an epitope of ADAM9 and an epitope of a molecule that is not ADAM9. In some embodiments, the bispecific antibody capable of simultaneously binding to ADAM9 and a second molecule that is not ADAM9 is produced using any of the methods described in PCT Publication Nos. WO 1998/002463, WO 2005/070966, WO 2006/107786 WO 2007/024715, WO 2007/075270, WO 2006/107617, WO 2007/046893, WO 2007/146968, WO 2008/003103, WO 2008/003116, WO 2008/027236, WO 2008/024188, WO 2009/132876, WO 2009/018386, WO 2010/028797, WO2010028796, WO 2010/028795, WO 2010/108127, WO 2010/136172, WO 2011/086091, WO 2011/133886, WO 2012/009544, WO 2013/003652, WO 2013/070565, WO 2012/162583, WO 2012/156430, WO 2013/174873, and WO 2014/022540, each of which is hereby incorporated herein by reference in its entirety.

### B. Bispecific Diabodies Lacking Fc Regions

One embodiment of the present invention relates to bispecific diabodies that are capable of binding to a first epitope and a second epitope, wherein the first epitope is an epitope of human ADAM9 and the second is an epitope of a molecule that is not ADAM9, preferably a molecule (e.g., CD2, CD3, CD8, CD16, T-Cell Receptor (TCR), NKG2D, etc.) present on the surface of an effector cell, such as a T lymphocyte, a natural killer (NK) cell or other mononuclear cell. Such diabodies comprise, and most preferably are composed of, a first polypeptide chain and a second polypeptide chain, whose sequences permit the polypeptide chains to covalently bind to each other to form a covalently associated diabody that is capable of simultaneously binding to an epitope of ADAM9 and the second epitope.

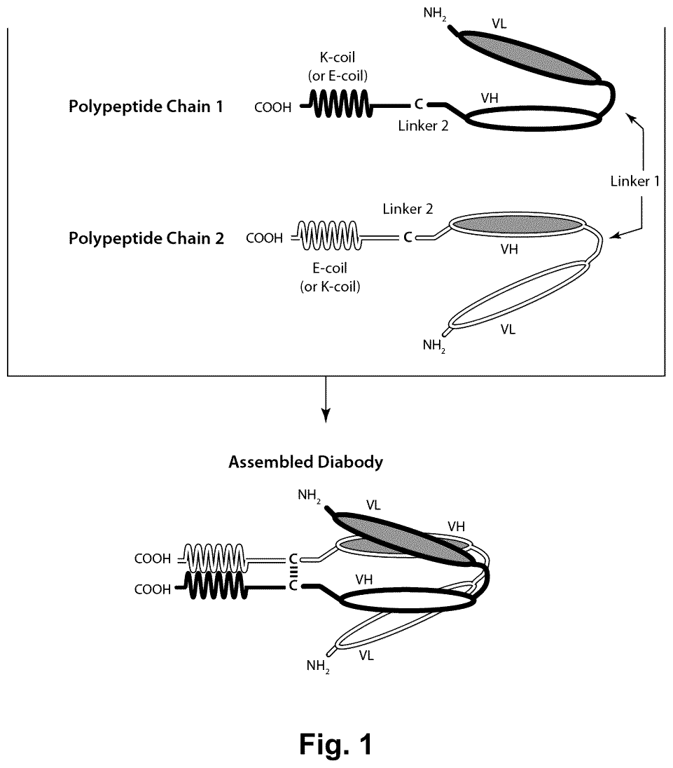

The first polypeptide chain of such an embodiment of bispecific diabodies comprises, in the N-terminal to C-terminal direction: an N-terminus, the VL Domain of a monoclonal antibody capable of binding to either the first or second epitope (i.e., either VL_{anti-ADAM9-VL} or VL_{Epitope 2}), a first intervening spacer peptide (Linker 1), a VH Domain of a monoclonal antibody capable of binding to either the second epitope (if such first polypeptide chain contains VL_{anti-ADAM9-VL}) or ADAM9 (if such first polypeptide chain contains VL_{Epitope 2}), a second intervening spacer peptide (Linker 2) optionally containing a cysteine residue, a Heterodimer-Promoting Domain and a C-terminus (FIG. 1).

The second polypeptide chain of this embodiment of bispecific diabodies comprises, in the N-terminal to C-terminal direction: an N-terminus, a VL Domain of a monoclonal antibody capable of binding to either the first or second epitope (i.e., either VL_{anti-ADAM9-VL} or VL_{Epitope 2}, and being the VL Domain not selected for inclusion in the first polypeptide chain of the diabody), an intervening spacer peptide (Linker 1), a VH Domain of a monoclonal antibody capable of binding to either the second epitope (if such second polypeptide chain contains VL_{anti-ADAM9-VL}) or to ADAM9 (if such second polypeptide chain contains VL_{Epitope 2}), a second intervening spacer peptide (Linker 2) optionally containing a cysteine residue, a Heterodimer-Promoting Domain, and a C-terminus (FIG. 1).

The VL Domain of the first polypeptide chain interacts with the VH Domain of the second polypeptide chain to form a first functional epitope-binding site that is specific for a first antigen (i.e., either ADAM9 or a molecule that contains the second epitope). Likewise, the VL Domain of the second polypeptide chain interacts with the VH Domain of the first polypeptide chain in order to form a second functional epitope-binding site that is specific for a second antigen (i.e., either the molecule that comprises the second epitope or ADAM9). Thus, the selection of the VL and VH Domains of the first and second polypeptide chains is coordinated, such that the two polypeptide chains of the diabody collectively comprise VL and VH Domains capable of binding to both an epitope of ADAM9 and to the second epitope (i.e., they collectively comprise VL_{anti-ADAM9-VL}/VH_{anti-ADAM9-VH} and VL_{Epitope 2}/VH_{Epitope 2}).

Most preferably, the length of the intervening spacer peptide (i.e., “Linker 1,” which separates such VL and VH Domains) is selected to substantially or completely prevent the VL and VH Domains of the polypeptide chain from binding to one another (for example consisting of from 0, 1, 2, 3, 4, 5, 6, 7, 8 or 9 intervening linker amino acid residues). Thus the VL and VH Domains of the first polypeptide chain are substantially or completely incapable of binding to one another. Likewise, the VL and VH Domains of the second polypeptide chain are substantially or completely incapable of binding to one another. A preferred intervening spacer peptide (Linker 1) has the sequence (SEQ ID NO:69): GGGSGGGG.

The length and composition of the second intervening spacer peptide (“Linker 2”) is selected based on the choice of one or more polypeptide domains that promote such dimerization (i.e., a “Heterodimer-Promoting Domain”). Typically, the second intervening spacer peptide (Linker 2) will comprise 3-20 amino acid residues. In particular, where the employed Heterodimer-Promoting Domain(s) do/does not comprise a cysteine residue a cysteine-containing second intervening spacer peptide (Linker 2) is utilized. A cysteine-containing second intervening spacer peptide (Linker 2) will contain 1, 2, 3 or more cysteines. A preferred cysteine-containing spacer peptide (Linker 2) has the sequence GGCGGG (SEQ ID NO:70). Alternatively, Linker 2 does not comprise a cysteine (e.g., GGG, GGGS (SEQ ID NO:71), LGGGSG (SEQ ID NO:72), GGGSGGGSGGG (SEQ ID NO:73), ASTKG (SEQ ID NO:74), LEPKSS (SEQ ID NO:75), APSSS (SEQ ID NO:76), etc.) and a Cysteine-Containing Heterodimer-Promoting Domain, as described below is used. Optionally, both a cysteine-containing Linker 2 and a cysteine-containing Heterodimer-Promoting Domain are used.

The Heterodimer-Promoting Domains may be GVEPKSC (SEQ ID NO:77) or VEPKSC (SEQ ID NO:78) or AEPKSC (SEQ ID NO:79) on one polypeptide chain and GFNRGEC (SEQ ID NO:80) or FNRGEC (SEQ ID NO:81) on the other polypeptide chain (see, U.S. Pat. No. 9,296,816).

In a preferred embodiment, the Heterodimer-Promoting Domains will comprise tandemly repeated coil domains of opposing charge for example, “E-coil” helical domains (SEQ ID NO:82): EVAALEK-EVAALEK-EVAALEK-EVAALEK), whose glutamate residues will form a negative charge at pH 7, and “K-coil” domains (SEQ ID NO:83): KVAALKE-KVAALKE-KVAALKE-KVAALKE), whose lysine residues will form a positive charge at pH 7. The presence of such charged domains promotes association between the first and second polypeptides, and thus fosters heterodimer formation. Heterodimer-Promoting Domains that comprise modifications of the above-described E-coil and K-coil sequences so as to include one or more cysteine residues may be utilized. The presence of such cysteine residues permits the coil present on one polypeptide chain to become covalently bonded to a complementary coil present on another polypeptide chain, thereby covalently bonding the polypeptide chains to one another and increasing the stability of the diabody. Examples of such particularly preferred are Heterodimer-Promoting Domains include a Modified E-Coil having the amino acid sequence EVAACEK-EVAALEK-EVAALEK-EVAALEK (SEQ ID NO:84), and a modified K-coil having the amino acid sequence KVAACKE-KVAALKE-KVAALKE-KVAALKE (SEQ ID NO:85).

As disclosed in PCT Publication No. WO 2012/018687, in order to improve the in vivo pharmacokinetic properties of diabodies, a diabody may be modified to contain a polypeptide portion of a serum-binding protein at one or more of the termini of the diabody. Most preferably, such polypeptide portion of a serum-binding protein will be installed at the C-terminus of a polypeptide chain of the diabody. Albumin is the most abundant protein in plasma and has a half-life of 19 days in humans. Albumin possesses several small molecule binding sites that permit it to non-covalently bind to other proteins and thereby extend their serum half-lives. The Albumin-Binding Domain 3 (ABD3) of protein G of Streptococcus strain G148 consists of 46 amino acid residues forming a stable three-helix bundle and has broad albumin-binding specificity (Johansson, M. U. et al. (2002) “Structure, Specificity, And Mode Of Interaction For Bacterial Albumin-Binding Modules,” J. Biol. Chem. 277(10):8114-8120). Thus, a particularly preferred polypeptide portion of a serum-binding protein for improving the in vivo pharmacokinetic properties of a diabody is the Albumin-Binding Domain (ABD) from streptococcal protein G, and more preferably, the Albumin-Binding Domain 3 (ABD3) of Protein G of Streptococcus strain G148 (SEQ ID NO:86): LAEAKVLANR ELDKYGVSDY YKNLIDNAKS AEGVKALIDE ILAALP.

As disclosed in PCT Publication No. WO 2012/162068 (herein incorporated by reference), “deimmunized” variants of SEQ ID NO:86 have the ability to attenuate or eliminate MHC class II binding. Based on combinational mutation results, the following combinations of substitutions are considered to be preferred substitutions for forming such a deimmunized ABD: 66D/70S+71A; 66S/70S+71A; 66S/70S+79A; 64A/65A/71A; 64A/65A/71A+66S; 64A/65A/71A+66D; 64A/65A/71A+66E; 64A/65A/79A+66S; 64A/65A/79A+66D; 64A/65A/79A+66E. Variant ABDs having the modifications L64A, I65A and D79A or the modifications N66S, T70S and D79A. Variant deimmunized ABD having the amino acid sequence:

(SEQ ID NO: 87)
LAEAKVLANR ELDKYGVSDY YKNLID₆₆NAKS₇₀ A₇₁EGVKALIDE
ILAALP,

or the amino acid sequence:

(SEQ ID NO: 88)
LAEAKVLANR ELDKYGVSDY YKNA₆₄A₆₅NNAKT VEGVKALIA₇₉E
ILAALP,

or the amino acid sequence:

(SEQ ID NO: 89)
LAEAKVLANR ELDKYGVSDY YKNLIS₆₆NAKS₇₀ VEGVKALIA₇₉E
ILAALP,

are particularly preferred as such deimmunized ABD exhibit substantially wild-type binding while providing attenuated MHC class II binding. Thus, the first polypeptide chain of such a diabody having an ABD contains a third linker (Linker 3) preferably positioned C-terminally to the E-coil (or K-coil) Domain of such polypeptide chain so as to intervene between the E-coil (or K-coil) Domain and the ABD (which is preferably a deimmunized ABD). A preferred sequence for such Linker 3 is GGGS (SEQ ID NO:71).

### C. Multispecific Diabodies Containing Fc Regions

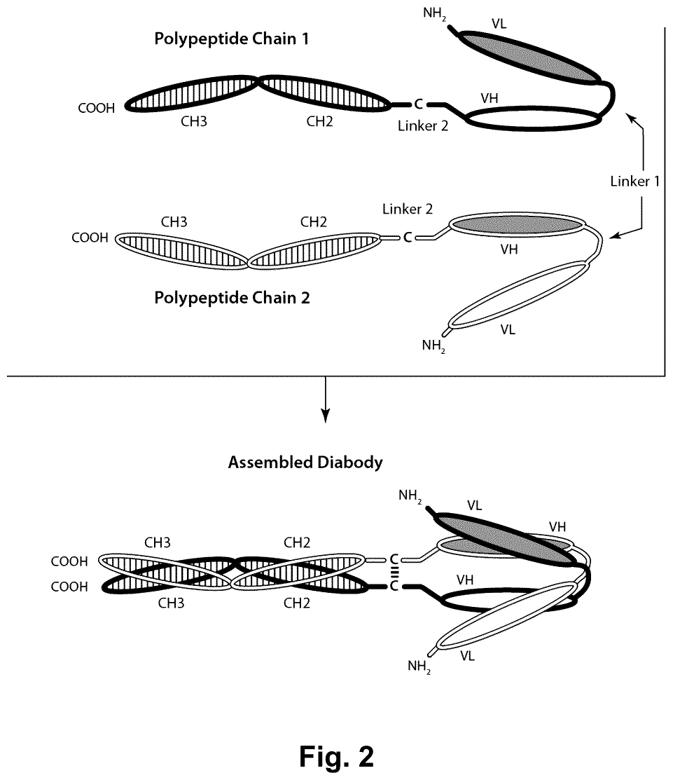

One embodiment of the present invention relates to multispecific diabodies capable of simultaneously binding to an epitope of ADAM9 and a second epitope (i.e., a different epitope of ADAM9 or an epitope of a molecule that is not ADAM9) that comprise an Fc Region. The addition of an IgG CH2-CH3 Domain to one or both of the diabody polypeptide chains, such that the complexing of the diabody chains results in the formation of an Fc Region, increases the biological half-life and/or alters the valency of the diabody. Such diabodies comprise, two or more polypeptide chains whose sequences permit the polypeptide chains to covalently bind to each other to form a covalently associated diabody that is capable of simultaneously binding to an epitope of ADAM9 and the second epitope. Incorporating an IgG CH2-CH3 Domains onto both of the diabody polypeptides will permit a two-chain bispecific Fc-Region-containing diabody to form (FIG. 2).

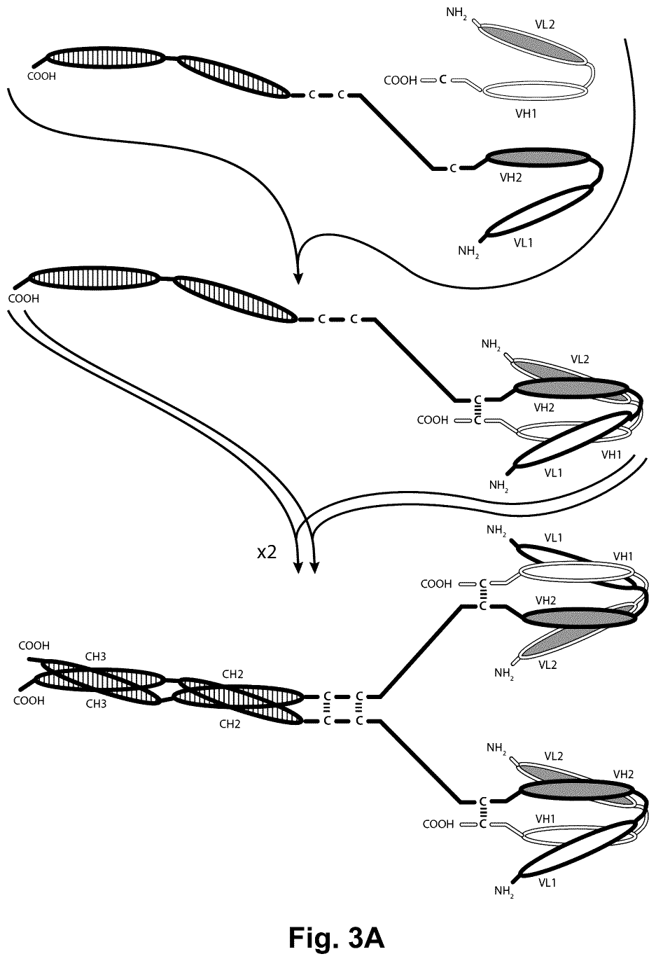

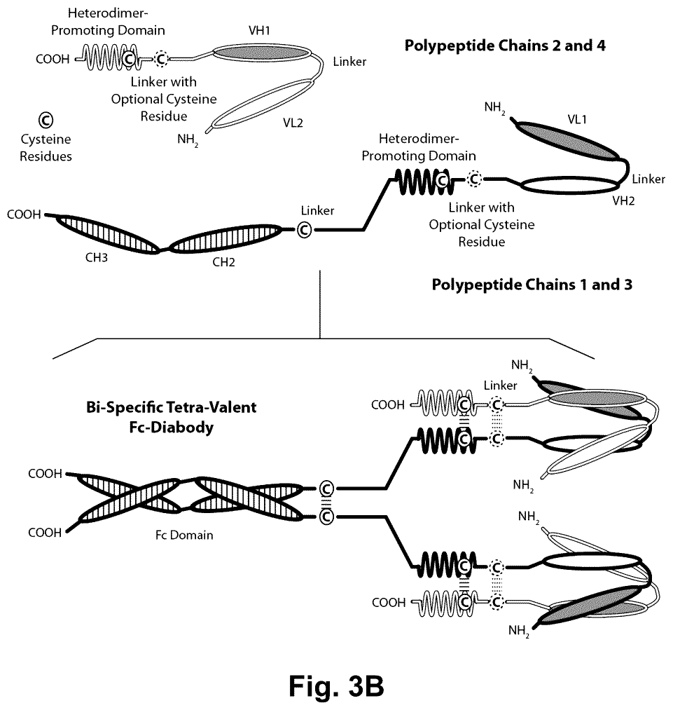

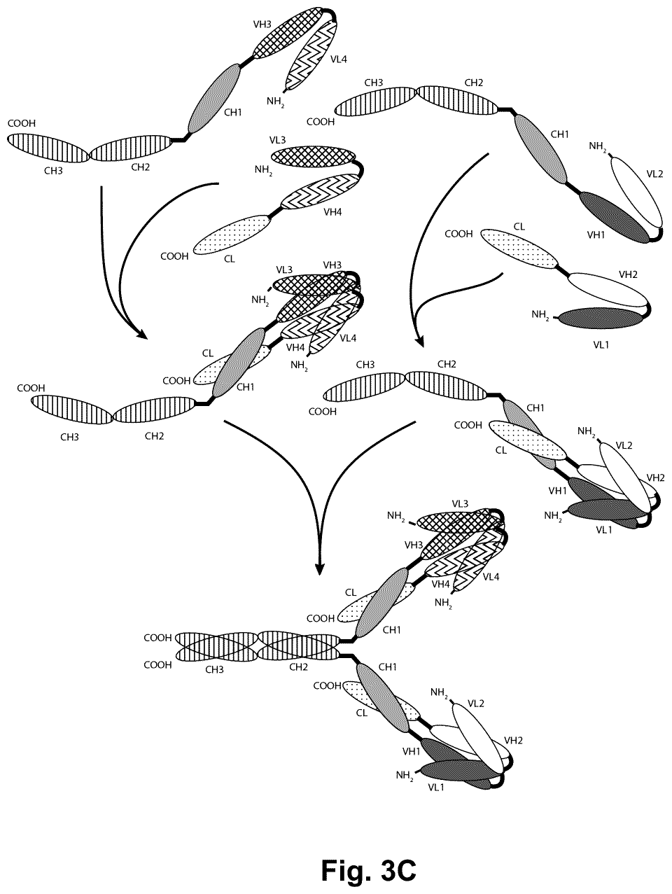

Alternatively, incorporating an IgG CH2-CH3 Domains onto only one of the diabody polypeptides will permit a more complex four-chain bispecific Fc Region-containing diabody to form (FIGS. 3A-3C). FIG. 3C shows a representative four-chain diabody possessing the Constant Light (CL) Domain and the Constant Heavy CH1 Domain, however fragments of such domains as well as other polypeptides may alternatively be employed (see, e.g., FIGS. 3A and 3B, US Patent Publication Nos. 2013-0295121; 2010-0174053 and 2009-0060910; European Patent Publication Nos. EP 2714079; EP 2601216; EP 2376109; EP 2158221 and PCT Publication Nos. WO 2012/162068; WO 2012/018687; WO 2010/080538). Thus, for example, in lieu of the CH1 Domain, one may employ a peptide having the amino acid sequence GVEPKSC (SEQ ID NO:77), VEPKSC (SEQ ID NO:78), or AEPKSC (SEQ ID NO:79), derived from the Hinge Region of a human IgG, and in lieu of the CL Domain, one may employ the C-terminal 6 amino acids of the human kappa light chain, GFNRGEC (SEQ ID NO:80) or FNRGEC (SEQ ID NO:81). A representative peptide containing four-chain diabody is shown in FIG. 3A. Alternatively, or in addition, one may employ a peptide comprising tandem coil domains of opposing charge such as the “E-coil” helical domains (SEQ ID NO:82): EVAALEK-EVAALEK-EVAALEK-EVAALEK or SEQ ID NO:84): EVAACEK-EVAALEK-EVAALEK-EVAALEK); and the “K-coil” domains (SEQ ID NO:83): KVAALKE-KVAALKE-KVAALKE-KVAALKE or SEQ ID NO:85): KVAACKE-KVAALKE-KVAALKE-KVAALKE). A representative coil domain containing four-chain diabody is shown in FIG. 3B.

The Fc Region-containing molecules of the present invention may include additional intervening spacer peptides (Linkers), generally such Linkers will be incorporated between a Heterodimer-Promoting Domain (e.g., an E-coil or K-coil) and a CH2-CH3 Domain and/or between a CH2-CH3 Domain and a Variable Domain (i.e., VH or VL). Typically, the additional Linkers will comprise 3-20 amino acid residues and may optionally contain all or a portion of an IgG Hinge Region (preferably a cysteine-containing portion of an IgG Hinge Region). Linkers that may be employed in the bispecific Fc Region-containing diabody molecules of the present invention include: GGGS (SEQ ID NO:71), LGGGSG (SEQ ID NO:72), GGGSGGGSGGG (SEQ ID NO:73), ASTKG (SEQ ID NO:74), LEPKSS (SEQ ID NO:75), APSSS (SEQ ID NO:76), APSSSPME (SEQ ID NO:90), VEPKSADKTHTCPPCP (SEQ ID NO:91), LEPKSADKTHTCPPC (SEQ ID NO:92), DKTHTCPPCP (SEQ ID NO:93), GGC, and GGG. LEPKSS (SEQ ID NO:75) may be used in lieu of GGG or GGC for ease of cloning. Additionally, the amino acids GGG, or LE PKSS (SEQ ID NO:75) may be immediately followed by DKTHTCPPCP (SEQ ID NO:93) to form the alternate linkers: GGGDKTHTCPPCP (SEQ ID NO:94); and LEPKSSDKTHTCPPCP (SEQ ID NO:95). Bispecific Fc Region-containing molecules of the present invention may incorporate an IgG Hinge Region in addition to or in place of a linker. Exemplary Hinge Regions include: EPKSCDKTHTCPPCP (SEQ ID NO:96) from IgG1, ERKCCVECPPCP (SEQ ID NO:97) from IgG2, ELKTPLGDTT HTCPRCPEPK SCDTPPPCPR CPEPKSCDTP PPCPRCPEPK SCDTPPPCPR CP (SEQ ID NO:206) from IgG3, ESKYGPPCPSCP (SEQ ID NO:98) from IgG4, and ESKYGPPCPPCP (SEQ ID NO:99), which is an IgG4 hinge variant comprising a stabilizing S228P substitution (underlined) (as numbered by the EU index as set forth in Kabat) to reduce strand exchange.

As provided in FIG. 3A-3C, Fc Region-containing diabodies of the invention may comprise four chains. The first and third polypeptide chains of such a diabody contain three domains: (i) a VL1-containing Domain, (ii) a VH2-containing Domain, (iii) a Heterodimer-Promoting Domain, and (iv) a Domain containing a CH2-CH3 sequence. The second and fourth polypeptide chains contain: (i) a VL2-containing Domain, (ii) a VH1-containing Domain, and (iii) a Heterodimer-Promoting Domain, where the Heterodimer-Promoting Domains promote the dimerization of the first/third polypeptide chains with the second/fourth polypeptide chains. The VL and/or VH Domains of the third and fourth polypeptide chains, and VL and/or VH Domains of the first and second polypeptide chains may be the same or different so as to permit tetravalent binding that is either monospecific, bispecific or tetraspecific. The notation “VL3” and “VH3” denote respectively, the Light Chain Variable Domain and Heavy Chain Variable Domain that bind a “third” epitope of such diabody. Similarly, the notation “VL4” and “VH4” denote respectively, the Light Chain Variable Domain and Heavy Chain Variable Domain that bind a “fourth” epitope of such diabody. The general structure of the polypeptide chains of a representative four-chain bispecific Fc Region-containing diabodies of invention is provided in Table 2:

TABLE 2

| | | |
|---|---|---|
| Bispecific | 2^{nd} Chain | NH₂—VL2—VH1—HPD—COOH |
| | 1^{st} Chain | NH₂—VL1—VH2—HPD—CH2— |
| | | CH3—COOH |
| | 1^{st} Chain | NH₂—VL1—VH2—HPD—CH2— |
| | | CH3—COOH |
| | 2^{nd} Chain | NH₂—VL2—VH1—HPD—COOH |
| Tetraspecific | 2^{nd} Chain | NH₂—VL2—VH1—HPD—COOH |
| | 1^{st} Chain | NH₂—VL1—VH2—HPD—CH2— |
| | | CH3—COOH |
| | 3^{rd} Chain | NH₂—VL3—VH4—HPD—CH2— |
| | | CH3—COOH |
| | 4^{th} Chain | NH₂—VL4—VH3—HPD—COOH |

HPD = Heterodimer-Promoting Domain

In a specific embodiment, diabodies of the present invention are bispecific, tetravalent (i.e., possess four epitope-binding sites), Fc-containing diabodies that are composed of four total polypeptide chains (FIGS. 3A-3C). The bispecific, tetravalent, Fc-containing diabodies of the invention comprise two epitope-binding sites immunospecific for ADAM9 (which may be capable of binding to the same epitope of ADAM9 or to different epitopes of ADAM9), and two epitope-binding sites immunospecific for a second molecule (which may be capable of binding to the same epitope of the second molecule or to different epitopes of the second molecule). Preferably, the second molecule is a molecule (e.g., CD2, CD3, CD8, CD16, T-Cell Receptor (TCR), NKG2D, etc.) present on the surface of an effector cell, such as a T lymphocyte, a natural killer (NK) cell or other mononuclear cell.

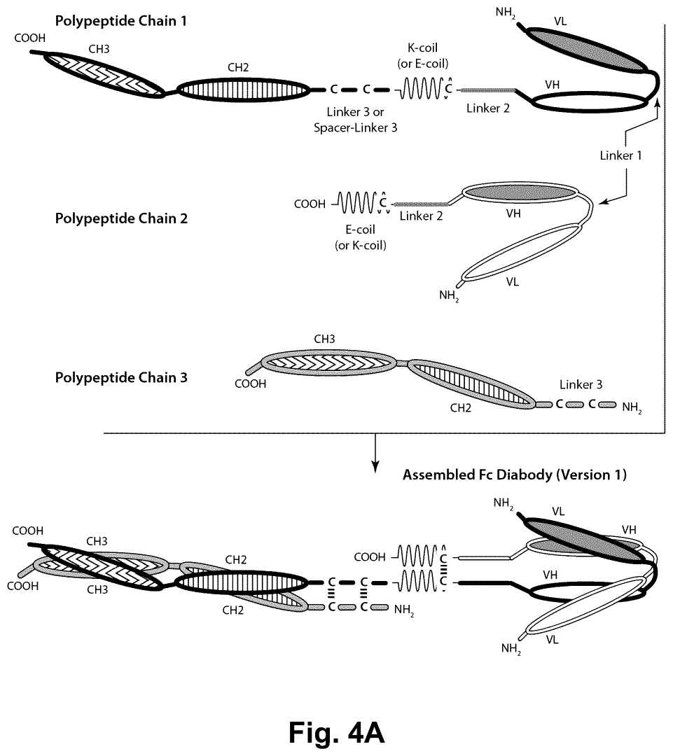

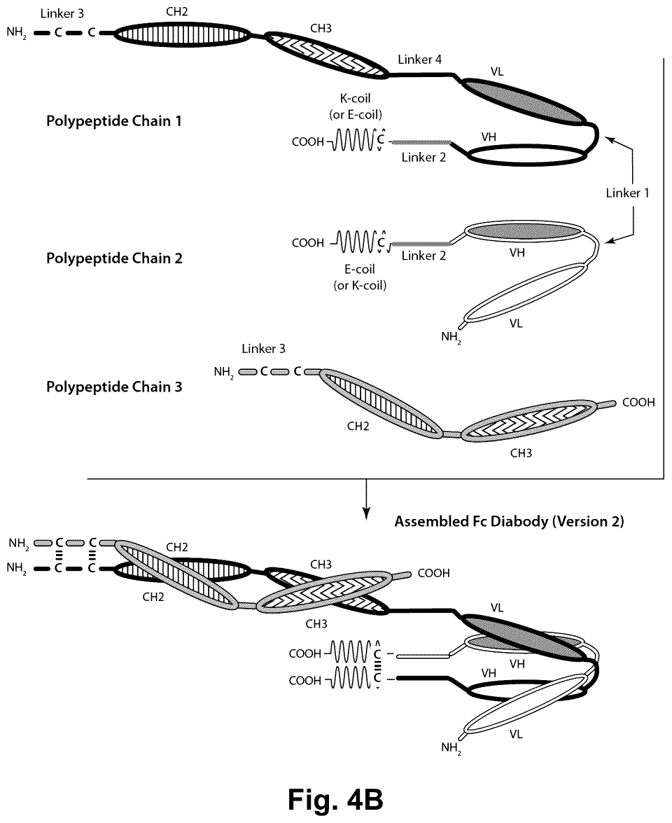

In a further embodiment, the Fc Region-containing diabodies of the present invention may comprise three polypeptide chains. The first polypeptide of such a diabody contains three domains: (i) a VL1-containing Domain, (ii) a VH2-containing Domain and (iii) a Domain containing a CH2-CH3 sequence. The second polypeptide of such a diabody contains: (i) a VL2-containing Domain, (ii) a VH1-containing Domain and (iii) a Domain that promotes heterodimerization and covalent bonding with the diabody's first polypeptide chain. The third polypeptide of such a diabody comprises a CH2-CH3 sequence. Thus, the first and second polypeptide chains of such a diabody associate together to form a VL1/VH1 epitope-binding site that is capable of binding to a first antigen (i.e., either ADAM9 or a molecule that comprises a second epitope), as well as a VL2/VH2 epitope-binding site that is capable of binding to a second antigen (i.e., either the molecule that contains the second epitope or ADAM9). The first and second polypeptides are bonded to one another through a disulfide bond involving cysteine residues in their respective Third Domains. Notably, the first and third polypeptide chains complex with one another to form an Fc Region that is stabilized via a disulfide bond. Such bispecific diabodies have enhanced potency. FIGS. 4A and 4B illustrate the structures of such diabodies. Such Fc-Region-containing diabodies may have either of two orientations (Table 3):

TABLE 3

| | | |
|---|---|---|
| First | 3^{rd} Chain | NH₂—CH2—CH3—COOH |
| Orientation | 1^{st} Chain | NH₂—VL1—VH2—HPD— |
| | | CH2—CH3—COOH |
| | 2^{nd} Chain | NH₂—VL2—VH1—HPD—COOH |
| Second | 3^{rd} Chain | NH₂—CH2—CH3—COOH |
| Orientation | 1^{st} Chain | NH₂—CH2—CH3—VL1— |
| | | VH2—HPD—COOH |
| | 2^{nd} Chain | NH₂—VL2—VH1—HPD—COOH |

HPD = Heterodimer-Promoting Domain

In a specific embodiment, diabodies of the present invention are bispecific, bivalent (i.e., possess two epitope-binding sites), Fc-containing diabodies that are composed of three total polypeptide chains (FIGS. 4A-4B). The bispecific, bivalent Fc-containing diabodies of the invention comprise one epitope-binding site immunospecific for ADAM9, and one epitope-binding site immunospecific for a second molecule. Preferably, the second molecule is a molecule (e.g., CD2, CD3, CD8, CD16, T-Cell Receptor (TCR), NKG2D, etc.) present on the surface of an effector cell, such as a T lymphocyte, a natural killer (NK) cell or other mononuclear cell.

In a further embodiment, the Fc Region-containing diabodies may comprise a total of five polypeptide chains. In a particular embodiment, two of said five polypeptide chains have the same amino acid sequence. The first polypeptide chain of such a diabody contains: (i) a VH1-containing domain, (ii) a CH1-containing domain, and (iii) a Domain containing a CH2-CH3 sequence. The first polypeptide chain may be the heavy chain of an antibody that contains a VH1 and a heavy chain constant region. The second and fifth polypeptide chains of such a diabody contain: (i) a VL1-containing domain, and (ii) a CL-containing domain. The second and/or fifth polypeptide chains of such a diabody may be light chains of an antibody that contains a VL1 complementary to the VH1 of the first/third polypeptide chain. The first, second and/or fifth polypeptide chains may be isolated from a naturally occurring antibody. Alternatively, they may be constructed recombinantly. The third polypeptide chain of such a diabody contains: (i) a VH1-containing domain, (ii) a CH1-containing domain, (iii) a Domain containing a CH2-CH3 sequence, (iv) a VL2-containing Domain, (v) a VH3-containing Domain and (vi) a Heterodimer-Promoting Domain, where the Heterodimer-Promoting Domains promote the dimerization of the third chain with the fourth chain. The fourth polypeptide of such diabodies contains: (i) a VL3-containing Domain, (ii) a VH2-containing Domain and (iii) a Domain that promotes heterodimerization and covalent bonding with the diabody's third polypeptide chain.

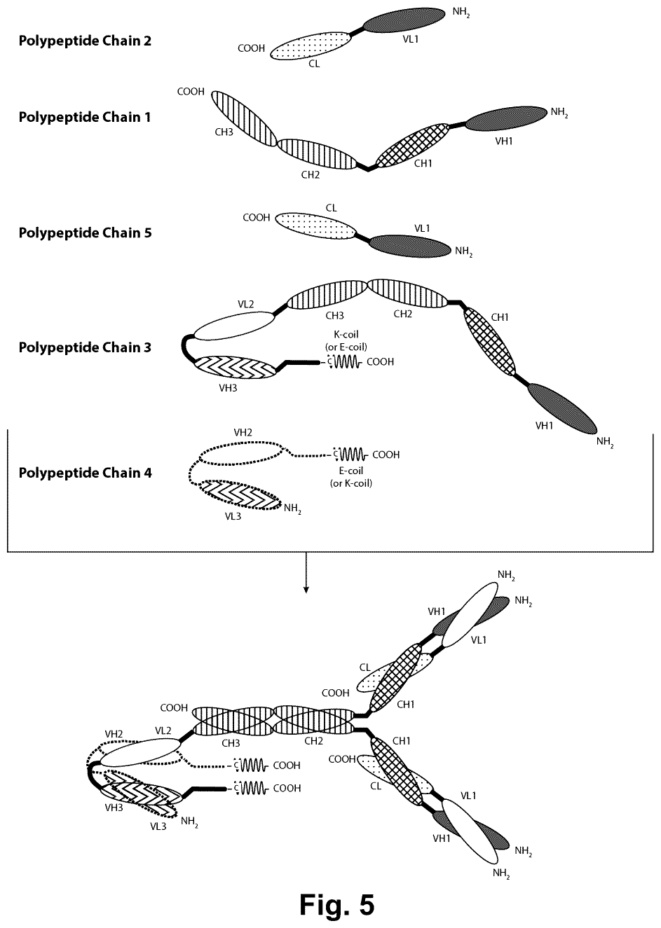

Thus, the first and second, and the third and fifth, polypeptide chains of such diabodies associate together to form two VL1/VH1 epitope-binding sites capable of binding a first epitope. The third and fourth polypeptide chains of such diabodies associate together to form a VL2/VH2 epitope-binding site that is capable of binding to a second epitope, as well as a VL3/VH3 binding site that is capable of binding to a third epitope. The first and third polypeptides are bonded to one another through a disulfide bond involving cysteine residues in their respective constant regions. Notably, the first and third polypeptide chains complex with one another to form an Fc Region. Such multispecific diabodies have enhanced potency. FIG. 5 illustrates the structure of such diabodies. It will be understood that the VL1/VH1, VL2/VH2, and VL3/VH3 Domains may be the same or different so as to permit binding that is monospecific, bispecific or trispecific. As provided herein, these domains are preferably selected so as to bind an epitope of ADAM9, an epitope of second molecule and optionally an epitope of a third molecule.

The VL and VH Domains of the polypeptide chains are selected so as to form VL/VH binding sites specific for a desired epitope. The VL/VH binding sites formed by the association of the polypeptide chains may be the same or different so as to permit tetravalent binding that is monospecific, bispecific, trispecific or tetraspecific. In particular, the VL and VH Domains maybe selected such that a multivalent diabody may comprise two binding sites for a first epitope and two binding sites for a second epitope, or three binding sites for a first epitope and one binding site for a second epitope, or two binding sites for a first epitope, one binding site for a second epitope and one binding site for a third epitope (as depicted in FIG. 5). The general structure of the polypeptide chains of representative five-chain Fc Region-containing diabodies of invention is provided in Table 4:

TABLE 4

| | | |
|---|---|---|
| Bispecific | 2^{nd} Chain | NH₂—VL1—CL—COOH |
| (2 × 2) | 1^{st} Chain | NH₂—VL1—CH1—CH2—CH3—COOH |
| | 3^{rd} Chain | NH₂—VH1—CH1—CH2—CH3—VL2—VH2—HPD—COOH |
| | 5^{nd} Chain | NH₂—VL1—CL—COOH |
| | 4^{th} Chain | NH₂—VL2—VH2—HPD—COOH |
| Bispecific (3 × 1) | 2^{nd} Chain | NH₂—VL1—CL—COOH |
| | 1^{st} Chain | NH₂—VH1—CH1—CH2—CH3—COOH |
| | 3^{rd} Chain | NH₂—VH1—CH1—CH2—CH3—VL1—VH2—HPD—COOH |
| | 5^{nd} Chain | NH₂—VL1—CL—COOH |
| | 4^{th} Chain | NH₂—VL2—VL1—HPD—COOH |
| Trispecific | 2^{nd} Chain | NH₂—VL1—CL—COOH |
| (2 × 1 × 1) | 1^{st} Chain | NH₂—VL1—CH1—CH2—CH3—COOH |
| | 3^{rd} Chain | NH₂—VH1—CH1—CH2—CH3—VL2—VH3—HPD—COOH |
| | 5^{nd} Chain | NH₂—VL1—CL—COOH |
| | 4^{th} Chain | NH₂—VL3—VH2—HPD—COOH |

HPD = Heterodimer-Promoting Domain

In a specific embodiment, diabodies of the present invention are bispecific, tetravalent (i.e., possess four epitope-binding sites), Fe-containing diabodies that are composed of five total polypeptide chains having two epitope-binding sites immunospecific for ADAM9 (which may be capable of binding to the same epitope of ADAM9 or to different epitopes of ADAM9), and two epitope-binding sites specific for a second molecule (which may be capable of binding to the same epitope of the second molecule or to different epitopes of the second molecule). In another embodiment, the bispecific, tetravalent, Fc-containing diabodies of the invention comprise three epitope-binding sites immunospecific for ADAM9 (which may be capable of binding to the same epitope of ADAM9 or to two or three different epitopes of ADAM9), and one epitope-binding site specific for a second molecule. In another embodiment, the bispecific, tetravalent, Fc-containing diabodies of the invention comprise one epitope-binding site immunospecific for ADAM9, and three epitope-binding sites specific for a second molecule (which may be capable of binding to the same epitope of the second molecule or to two or three different epitopes of the second molecule). As provided above, the VL and VH domains may be selected to permit trispecific binding. Accordingly, the invention also encompasses trispecific, tetravalent, Fc-containing diabodies. The trispecific, tetravalent, Fc-containing diabodies of the invention comprise two epitope-binding sites immunospecific for ADAM9, one epitope-binding site immunospecific for a second molecule, and one epitope-binding site immunospecific for a third molecule. In certain embodiments, the second molecule is a molecule (e.g., CD2, CD3, CD8, CD16, T-Cell Receptor (TCR), NKG2D, etc.) present on the surface of an effector cell, such as a T lymphocyte, a natural killer (NK) cell or other mononuclear cell. In certain embodiments, the second molecule is CD3 and the third molecule is CD8.

### D. Trivalent Binding Molecules Containing Fc Regions

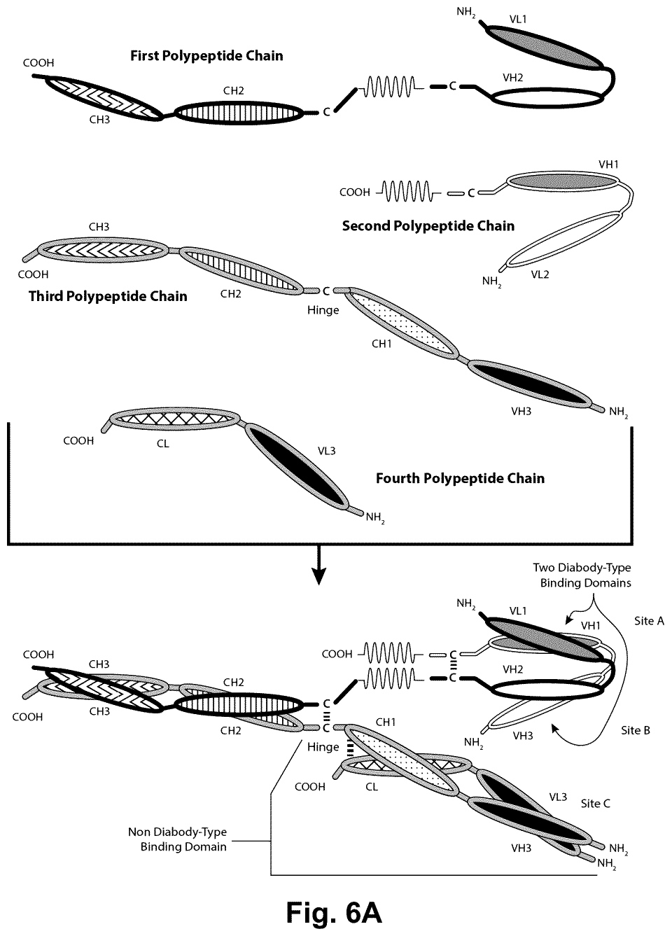

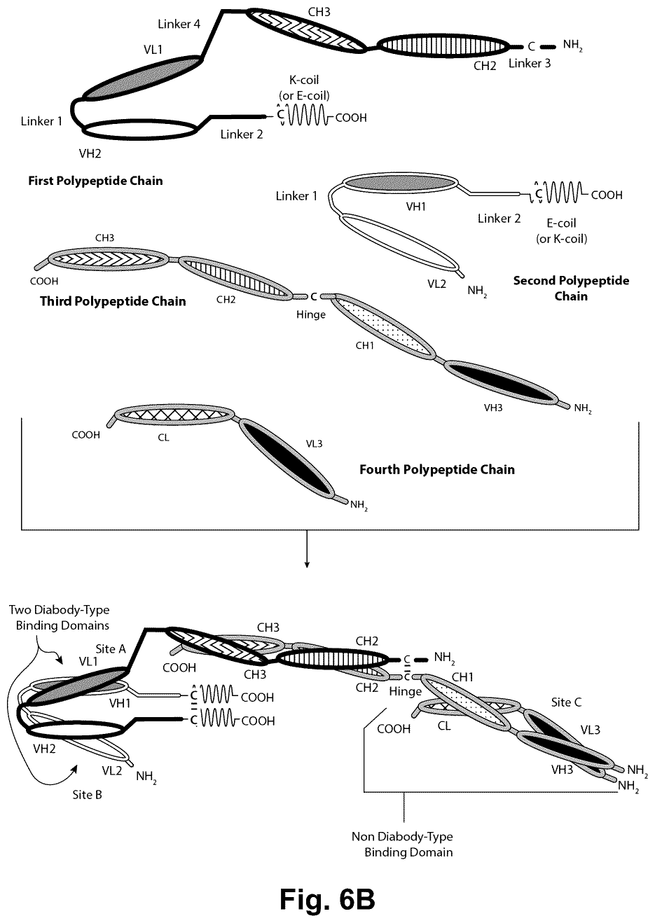

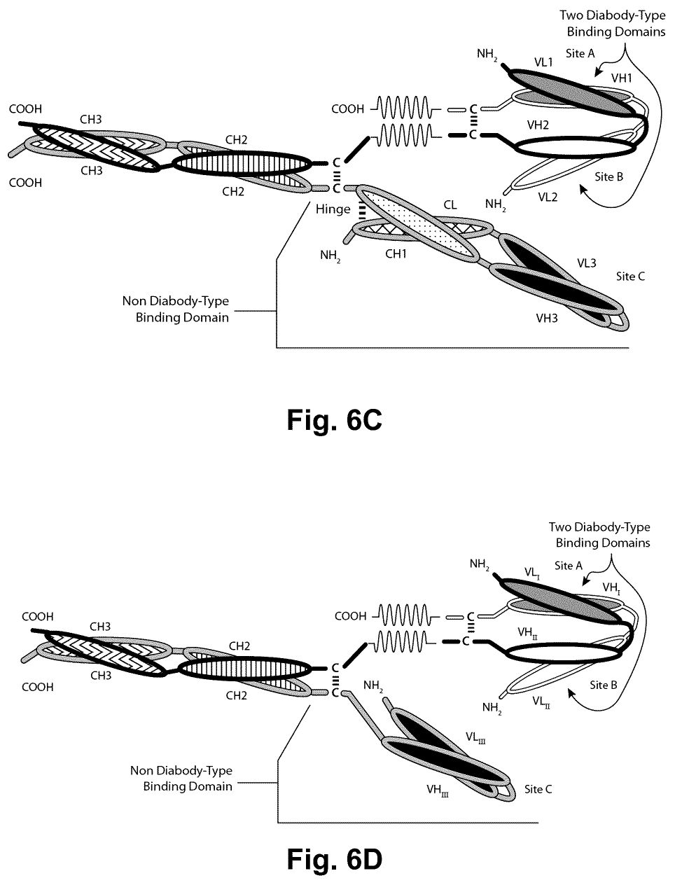

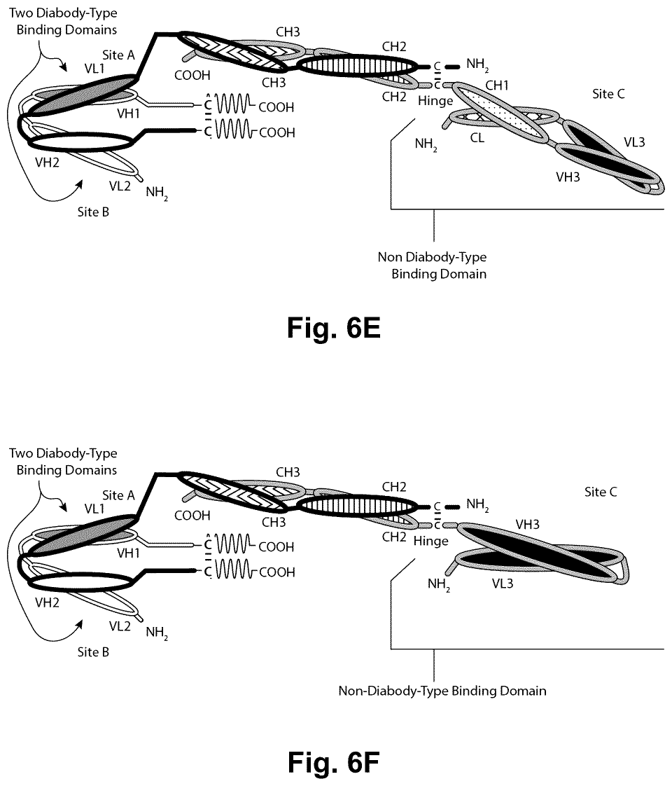

A further embodiment of the present invention relates to trivalent binding molecules comprising an Fc Region capable of simultaneously binding a first epitope, a second epitope and a third epitope, wherein at least one of such epitopes is not identical to another. Such trivalent binding molecules comprise three epitope-binding sites, two of which are Diabody-Type Binding Domains, which provide binding Site A and binding Site B, and one of which is a Fab-Type Binding Domain, or an scFv-Type Binding Domain, which provides binding Site C (see, e.g., FIGS. 6A-6F, and PCT Publication Nos: WO 2015/184207; and WO 2015/184203). Such trivalent binding molecules thus comprise “VL1”/“VH1” domains that are capable of binding to the first epitope and “VL2”/“VH2” domains that are capable of binding to the second epitope and “VL3” and “VH3” domains that are capable of binding to the “third” epitope of such trivalent binding molecule. A “Diabody-Type Binding Domain” is the type of epitope-binding site present in a diabody, and especially, a DART® diabody, as described above. Each of a “Fab-Type Binding Domain” and an “scFv-Type Binding Domain” are epitope-binding sites that are formed by the interaction of the VL Domain of an immunoglobulin light chain and a complementing VH Domain of an immunoglobulin heavy chain. Fab-Type Binding Domains differ from Diabody-Type Binding Domains in that the two polypeptide chains that form a Fab-Type Binding Domain comprise only a single epitope-binding site, whereas the two polypeptide chains that form a Diabody-Type Binding Domain comprise at least two epitope-binding sites. Similarly, scFv-Type Binding Domains also differ from Diabody-Type Binding Domains in that they comprise only a single epitope-binding site. Thus, as used herein Fab-Type, and scFv-Type Binding Domains are distinct from Diabody-Type Binding Domains.

Typically, the trivalent binding molecules of the present invention will comprise four different polypeptide chains (see FIGS. 6A-6B), however, the molecules may comprise fewer or greater numbers of polypeptide chains, for example by fusing such polypeptide chains to one another (e.g., via a peptide bond) or by dividing such polypeptide chains to form additional polypeptide chains, or by associating fewer or additional polypeptide chains via disulfide bonds. FIGS. 6C-6F illustrate this aspect of the present invention by schematically depicting such molecules having three polypeptide chains. As provided in FIGS. 6A-6F, the trivalent binding molecules of the present invention may have alternative orientations in which the Diabody-Type Binding Domains are N-terminal (FIGS. 6A, 6C and 6D) or C-terminal (FIGS. 6B, 6E and 6F) to an Fc Region.

In certain embodiments, the first polypeptide chain of such trivalent binding molecules of the present invention contains: (i) a VL1-containing Domain, (ii) a VH2-containing Domain, (iii) a Heterodimer-Promoting Domain, and (iv) a Domain containing a CH2-CH3 sequence. The VL1 and VL2 Domains are located N-terminal or C-terminal to the CH2-CH3-containing domain as presented in Table 4 (also see, FIGS. 6A and 6B). The second polypeptide chain of such embodiments contains: (i) a VL2-containing Domain, (ii) a VH1-containing Domain, and (iii) a Heterodimer-Promoting Domain. The third polypeptide chain of such embodiments contains: (i) a VH3-containing Domain, (ii) a CH1-containing Domain and (iii) a Domain containing a CH2-CH3 sequence. The third polypeptide chain may be the heavy chain of an antibody that contains a VH3 and a heavy chain constant region, or a polypeptide that contains such domains. The fourth polypeptide of such embodiments contains: (i) a VL3-containing Domain and (ii) a CL-containing Domain. The fourth polypeptide chains may be a light chain of an antibody that contains a VL3 complementary to the VH3 of the third polypeptide chain, or a polypeptide that contains such domains. The third or fourth polypeptide chains may be isolated from naturally occurring antibodies. Alternatively, they may be constructed recombinantly, synthetically or by other means.

The Light Chain Variable Domain of the first and second polypeptide chains are separated from the Heavy Chain Variable Domains of such polypeptide chains by an intervening spacer peptide having a length that is too short to permit their VL1/VH2 (or their VL2/VH1) domains to associate together to form epitope-binding site capable of binding to either the first or second epitope. A preferred intervening spacer peptide (Linker 1) for this purpose has the sequence (SEQ ID NO:69): GGGSGGGG. Other Domains of the trivalent binding molecules may be separated by one or more intervening spacer peptides (Linkers), optionally comprising a cysteine residue. In particular, as provided above, such Linkers will typically be incorporated between Variable Domains (i.e., VH or VL) and peptide Heterodimer-Promoting Domains (e.g., an E-coil or K-coil) and between such peptide Heterodimer-Promoting Domains (e.g., an E-coil or K-coil) and CH2-CH3 Domains. Exemplary linkers useful for the generation of trivalent binding molecules are provided above and are also provided in PCT Application Nos: PCT/US15/33081; and PCT/US15/33076. Thus, the first and second polypeptide chains of such trivalent binding molecules associate together to form a VL1/VH1 binding site capable of binding a first epitope, as well as a VL2/VH2 binding site that is capable of binding to a second epitope. The third and fourth polypeptide chains of such trivalent binding molecules associate together to form a VL3/VH3 binding site that is capable of binding to a third epitope.

As described above, the trivalent binding molecules of the present invention may comprise three polypeptides. Trivalent binding molecules comprising three polypeptide chains may be obtained by linking the domains of the fourth polypeptide N-terminal to the VH3-containing Domain of the third polypeptide (e.g., using an intervening spacer peptide (Linker 4)). Alternatively, a third polypeptide chain of a trivalent binding molecule of the invention containing the following domains is utilized: (i) a VL3-containing Domain, (ii) a VH3-containing Domain, and (iii) a Domain containing a CH2-CH3 sequence, wherein the VL3 and VH3 are spaced apart from one another by an intervening spacer peptide that is sufficiently long (at least 9 or more amino acid residues) so as to allow the association of these domains to form an epitope-binding site. One preferred intervening spacer peptide for this purpose has the sequence: GGGGSGGGGSGGGGS (SEQ ID NO:100).

It will be understood that the VL1/VH1, VL2/VH2, and VL3/VH3 Domains of such trivalent binding molecules may be different so as to permit binding that is monospecific, bispecific or trispecific. In particular, the VL and VH Domains may be selected such that a trivalent binding molecule comprises two binding sites for a first epitope and one binding sites for a second epitope, or one binding site for a first epitope and two binding sites for a second epitope, or one binding site for a first epitope, one binding site for a second epitope and one binding site for a third epitope.

However, as provided herein, these domains are preferably selected so as to bind an epitope of ADAM9, an epitope of second molecule, and an epitope of a third molecule. In certain embodiments, the second molecule is a molecule (e.g., CD2, CD3, CD8, CD16, T-Cell Receptor (TCR), NKG2D, etc.) present on the surface of an effector cell, such as a T lymphocyte, a natural killer (NK) cell or other mononuclear cell. In certain embodiments, the third molecule is CD8.

The general structure of the polypeptide chains of representative trivalent binding molecules of invention is provided in FIGS. 6A-6F and in Table 5:

TABLE 5

| | | |
|---|---|---|
| Four Chain | 2^{nd} Chain | NH₂—VL2—VH1—HPD—COOH |
| 1^{st} | 1^{st} Chain | NH₂—VL1—VH2—HPD—CH2— |
| Orientation | | CH3—COOH |
| | 3^{rd} Chain | NH₂—VH3—CH1—CH2—CH3—COOH |
| | 2^{nd} Chain | NH₂—VL3—CL—COOH |
| Four Chain | 2^{nd} Chain | NH₂—VL2—VH1—HPD—COOH |
| 2nd | 1^{st} Chain | NH₂—CH2—CH3—VL1—VH2— |
| Orientation | | HPD—COOH |
| | 3^{rd} Chain | NH₂—VH3—CH1—CH2—CH3—COOH |
| | 2^{nd} Chain | NH₂—VL3—CL—COOH |
| Three Chain | 2^{nd} Chain | NH₂—VL2—VH1—HPD—COOH |
| 1st | 1^{st} Chain | NH₂—VL1—VH2—HPD—CH2— |
| Orientation | | CH3—COOH |
| | 3^{rd} Chain | NH₂—VL3—VH3—HPD—CH2— |
| | | CH3—COOH |
| Three Chain | 2^{nd} Chain | NH₂—VL2—VL1—HPD—COOH |
| 2^{nd} | 1^{st} Chain | NH₂—CH2—CH3—VL1—VH2— |
| Orientation | | HPD—COOH |
| | 3^{rd} Chain | NH₂—VL3—VH3—HPD—CH2— |
| | | CH3—COOH |

HPD = Heterodimer-Promoting Domain

One embodiment of the present invention relates to trivalent binding molecules that comprise two epitope-binding sites for ADAM9 and one epitope-binding site for a second molecule. The two epitope-binding sites for ADAM9 may bind the same epitope or different epitopes. Another embodiment of the present invention relates to trivalent binding molecules that comprise, one epitope-binding site for ADAM9 and two epitope-binding sites for a second molecule. The two epitope-binding sites for the second molecule may bind the same epitope or different epitopes of the second molecule. A further embodiment of the present invention relates to trispecific trivalent binding molecules that comprise, one epitope-binding site for ADAM9, one epitope-binding site for a second molecule, and one epitope-binding site for a third molecule. In certain embodiments, the second molecule is a molecule (e.g., CD2, CD3, CD8, CD16, T-Cell Receptor (TCR), NKG2D, etc.) present on the surface of an effector cell, such as a T lymphocyte, a natural killer (NK) cell or other mononuclear cell. In certain embodiments, the second molecule is CD3 and the third molecule is CD8. As provided above, such trivalent binding molecules may comprise three, four, five, or more polypeptide chains.]]

### IX. Constant Domains and Variant Fc Regions

Provided herein are antibody “Constant Domains” useful in the generation of the ADAM9-binding molecules (e.g., antibodies, diabodies, trivalent binding molecules, etc.) of the invention.

A preferred CL Domain is a human IgG CL Kappa Domain. The amino acid sequence of an exemplary human CL Kappa Domain is (SEQ ID NO:101):

RTVAAPSVFI FPPSDEQLKS GTASVVCLLN NFYPREAKVQ
WKVDNALQSG NSQESVTEQD SKDSTYSLSS TLTLSKADYE
KHKVYACEVT HQGLSSPVTK SFNRGEC

Alternatively, an exemplary CL Domain is a human IgG CL Lambda Domain. The amino acid sequence of an exemplary human CL Lambda Domain is (SEQ ID NO:102):

QPKAAPSVTL FPPSSEELQA NKATLVCLIS DFYPGAVTVA
WKADSSPVKA GVETTPSKQS NNKYAASSYL SLTPEQWKSH
RSYSCQVTHE GSTVEKTVAP TECS

As provided herein, the ADAM9-binding molecules of the invention may comprise an Fc Region. The Fc Region of such molecules of the invention may be of any isotype (e.g., IgG1, IgG2, IgG3, or IgG4). The ADAM9-binding molecules of the invention may further comprise a CH1 Domain and/or a Hinge Region. When present, the CH1 Domain and/or Hinge Region may be of any isotype (e.g., IgG1, IgG2, IgG3, or IgG4), and is preferably of the same isotype as the desired Fc Region.

An exemplary CH1 Domain is a human IgG1 CH1 Domain. The amino acid sequence of an exemplary human IgG1 CH1 Domain is (SEQ ID NO:103):

ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKRV

An exemplary CH1 Domain is a human IgG2 CH1 Domain. The amino acid sequence of an exemplary human IgG2 CH1 Domain is (SEQ ID NO:104):

ASTKGPSVFP LAPCSRSTSE STAALGCLVK DYFPEPVTVS
WNSGALTSGV HTFPAVLQSS GLYSLSSVVT VPSSNFGTQT
YTCNVDHKPS NTKVDKTV

An exemplary CH1 Domain is a human IgG3 CH1 Domain. The amino acid sequence of an exemplary human IgG3 CH1 Domain is (SEQ ID NO:207):

ASTKGPSVFP LAPCSRSTSG GTAALGCLVK DYFPEPVTVS
WNSGALTSGV HTFPAVLQSS GLYSLSSVVT VPSSSLGTQT
YTCNVNHKPS NTKVDKRV

An exemplary CH1 Domain is a human IgG4 CH1 Domain. The amino acid sequence of an exemplary human IgG4 CH1 Domain is (SEQ ID NO:105):

ASTKGPSVFP LAPCSRSTSE STAALGCLVK DYFPEPVTVS
WNSGALTSGV HTFPAVLQSS GLYSLSSVVT VPSSSLGTKT
YTCNVDHKPS NTKVDKRV

One exemplary Hinge Region is a human IgG1 Hinge Region. The amino acid sequence of an exemplary human IgG1 Hinge Region is (SEQ ID NO:96):

EPKSCDKTHTCPPCP.

Another exemplary Hinge Region is a human IgG2 Hinge Region. The amino acid sequence of an exemplary human IgG2 Hinge Region is (SEQ ID NO:97):

ERKCCVECPPCP.

Another exemplary Hinge Region is a human IgG4 Hinge Region. The amino acid sequence of an exemplary human IgG4 Hinge Region is (SEQ ID NO:98): ESKYGPPCPSCP. As described above, an IgG4 Hinge Region may comprise a stabilizing mutation, such as the S228P substitution. The amino acid sequence of an exemplary stabilized IgG4 Hinge Region is (SEQ ID NO:99): ESKYGPPCPPCP.

The Fc Region of the Fc Region-containing molecules (e.g., antibodies, diabodies, trivalent binding molecules, etc.) of the present invention may be either a complete Fc Region (e.g., a complete IgG Fc Region) or only a fragment of an Fc Region. Optionally, the Fc Region of the Fc Region-containing molecules of the present invention lacks the C-terminal lysine amino acid residue.

In traditional immune function, the interaction of antibody-antigen complexes with cells of the immune system results in a wide array of responses, ranging from effector functions such as antibody dependent cytotoxicity, mast cell degranulation, and phagocytosis to immunomodulatory signals such as regulating lymphocyte proliferation and antibody secretion. All of these interactions are initiated through the binding of the Fc Region of antibodies or immune complexes to specialized cell surface receptors on hematopoietic cells. The diversity of cellular responses triggered by antibodies and immune complexes results from the structural heterogeneity of the three Fc receptors: FcγRI (CD64), FcγRII (CD32), and FcγRIII (CD16). FcγRI (CD64), FcγRIIA (CD32A) and FcγRIII (CD16) are activating (i.e., immune system enhancing) receptors; FcγRIIB (CD32B) is an inhibiting (i.e., immune system dampening) receptor. In addition, interaction with the neonatal Fc Receptor (FcRn) mediates the recycling of IgG molecules from the endosome to the cell surface and release into the blood. The amino acid sequence of exemplary wild-type IgG1 (SEQ ID NO:1), IgG2 (SEQ ID NO:2), IgG3 (SEQ ID NO:3), and IgG4 (SEQ ID NO:4) are presented above.

Modification of the Fc Region may lead to an altered phenotype, for example altered serum half-life, altered stability, altered susceptibility to cellular enzymes or altered effector function. It may therefore be desirable to modify an Fc Region-containing ADAM9-binding molecule of the present invention with respect to effector function, for example, so as to enhance the effectiveness of such molecule in treating cancer. Reduction or elimination of effector function is desirable in certain cases, for example in the case of antibodies whose mechanism of action involves blocking or antagonism, but not killing of the cells bearing a target antigen. Increased effector function is generally desirable when directed to undesirable cells, such as tumor and foreign cells, where the FcγRs are expressed at low levels, for example, tumor-specific B cells with low levels of FcγRIIB (e.g., non-Hodgkin's lymphoma, CLL, and Burkitt's lymphoma). Molecules of the invention possessing such conferred or altered effector function activity are useful for the treatment and/or prevention of a disease, disorder or infection in which an enhanced efficacy of effector function activity is desired.

Accordingly, in certain embodiments, the Fc Region of the Fc Region-containing molecules of the present invention may be an engineered variant Fc Region. Although the Fc Region of the bispecific Fc Region-containing molecules of the present invention may possess the ability to bind to one or more Fc receptors (e.g., FcγR(s)), more preferably such variant Fc Region have altered binding to FcγRIA (CD64), FcγRIIA (CD32A), FcγRIIB (CD32B), FcγRIIIA (CD16a) or FcγRIIIB (CD16b) (relative to the binding exhibited by a wild-type Fc Region), e.g., will have enhanced binding to an activating receptor and/or will have substantially reduced or no ability to bind to inhibitory receptor(s). Thus, the Fc Region of the Fc Region-containing molecules of the present invention may include some or all of the CH2 Domain and/or some or all of the CH3 Domain of a complete Fc Region, or may comprise a variant CH2 and/or a variant CH3 sequence (that may include, for example, one or more insertions and/or one or more deletions with respect to the CH2 or CH3 domains of a complete Fc Region). Such Fc Regions may comprise non-Fc polypeptide portions, or may comprise portions of non-naturally complete Fc Regions, or may comprise non-naturally occurring orientations of CH2 and/or CH3 Domains (such as, for example, two CH2 domains or two CH3 domains, or in the N-terminal to C-terminal direction, a CH3 Domain linked to a CH2 Domain, etc.).

Fc Region modifications identified as altering effector function are known in the art, including modifications that increase binding to activating receptors (e.g., FcγRIIA (CD16A) and reduce binding to inhibitory receptors (e.g., FcγRIIB (CD32B) (see, e.g., Stavenhagen, J. B. et al. (2007) “Fc Optimization Of Therapeutic Antibodies Enhances Their Ability To Kill Tumor Cells In Vitro And Controls Tumor Expansion In Vivo Via Low-Affinity Activating Fcgamma Receptors,” Cancer Res. 57(18):8882-8890). Table 6 lists exemplary single, double, triple, quadruple and quintuple substitutions (numbering is that of the EU index as in Kabat, and substitutions are relative to the amino acid sequence of SEQ ID NO:1) of exemplary modification that increase binding to activating receptors and/or reduce binding to inhibitory receptors.

TABLE 6
Variations of Preferred Activating Fc Regions
Single-Site Variations

| | | | |
|---|---|---|---|
| F243L | R292G | D270E | R292P |
| Y300L | P396L | | |

Double-Site Variations

| | | | |
|---|---|---|---|
| F243L and R292P | F243L and Y300L | F243L and P396L | R292P and Y300L |
| D270E and P396L | R292P and V305I | P396L and Q419H | P247L and N421K |
| R292P and P396L | Y300L and P396L | R255L and P396L | R292P and P305I |
| K392T and P396L | | | |

Triple-Site Variations

| | |
|---|---|
| F243L, P247L and N421K | P247L, D270E and N421K |
| F243L, R292P and Y300L | R255L, D270E and P396L |
| F243L, R292P and V305I | D270E, G316D and R416G |
| F243L, R292P and P396L | D270E, K392T and P396L |
| F243L, Y300L and P396L | D270E, P396L and Q419H |
| V284M, R292L and K370N | R292P, Y300L and P396L |

Quadruple-Site Variations

| | |
|---|---|
| L234F, F243L, R292P and Y300L | F243L, P247L, D270E and N421K |
| L234F, F243L, R292P and Y300L | F243L, R255L, D270E and P396L |
| L235I, F243L, R292P and Y300L | F243L, D270E, G316D and R416G |
| L235Q, F243L, R292P and Y300L | F243L, D270E, K392T and P396L |
| P247L, D270E, Y300L and N421K | F243L, R292P, Y300L, and P396L |
| R255L, D270E, R292G and P396L | F243L, R292P, V305I and P396L |
| R255L, D270E, Y300L and P396L | F243L, D270E, P396L and Q419H |
| D270E, G316D, P396L and R416G | |

Quintuple-Site Variations

| | |
|---|---|
| L235V, F243L, R292P, Y300L and P396L | F243L, R292P, V305I, Y300L and P396L |
| L235P, F243L, R292P, Y300L and P396L | |

Exemplary variants of human IgG1 Fc Regions with reduced binding to CD32B and/or increased binding to CD16A contain F243L, R292P, Y300L, V305I or P396L substitutions, wherein the numbering is that of the EU index as in Kabat. These amino acid substitutions may be present in a human IgG1 Fc Region in any combination. In one embodiment, the variant human IgG1 Fc Region contains a F243L, R292P and Y300L substitution. In another embodiment, the variant human IgG1 Fc Region contains a F243L, R292P, Y300L, V305I and P396L substitution.

In certain embodiments, it is preferred for the Fc Regions of ADAM9-binding molecules of the present invention to exhibit decreased (or substantially no) binding to FcγRIA (CD64), FcγRIIA (CD32A), FcγRIIB (CD32B), FcγRIIIA (CD16a) or FcγRIIIB (CD16b) (relative to the binding exhibited by the wild-type IgG1 Fc Region (SEQ ID NO:1). In a specific embodiment, the ADAM9-binding molecules of the present invention comprise an IgG Fc Region that exhibits reduced ADCC effector function. In a preferred embodiment the CH2-CH3 Domains of such ADAM9-binding molecules include any 1, 2, 3, or 4 of the substitutions: L234A, L235A, D265A, N297Q, and N297G, wherein the numbering is that of the EU index as in Kabat. In another embodiment, the CH2-CH3 Domains contain an N297Q substitution, an N297G substitution, L234A and L235A substitutions or a D265A substitution, as these mutations abolish FcR binding. Alternatively, a CH2-CH3 Domain of a naturally occurring Fc region that inherently exhibits decreased (or substantially no) binding to FcγRIIIA (CD16a) and/or reduced effector function (relative to the binding and effector function exhibited by the wild-type IgG1 Fc Region (SEQ ID NO:1)) is utilized. In a specific embodiment, the ADAM9-binding molecules of the present invention comprise an IgG2 Fc Region (SEQ ID NO:2) or an IgG4 Fc Region (SEQ ID:NO:4). When an IgG4 Fc Region is utilized, the instant invention also encompasses the introduction of a stabilizing mutation, such as the Hinge Region S228P substitution described above (see, e.g., SEQ ID NO:99). Since the N297G, N297Q, L234A, L235A and D265A substitutions abolish effector function, in circumstances in which effector function is desired, these substitutions would preferably not be employed.

A preferred IgG1 sequence for the CH2 and CH3 Domains of the Fc Region-containing molecules of the present invention having reduced or abolished effector function will comprise the substitutions L234A/L235A (shown underlined) (SEQ ID NO:106):

APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLTCLVK GFYPSDIAVE WESNGQPENN
YKTTPPVLDS DGSFFLYSKL TVDKSRWQQG NVFSCSVMHE
ALHNHYTQKS LSLSPGX

wherein X is a lysine (K) or is absent.

A second preferred IgG1 sequence for the CH2 and CH3 Domains of the Fc Region-containing molecules of the present invention comprises an S442C substitution (shown underlined), so as to permit two CH3 domains to be covalently bonded to one another via a disulfide bond or to permit conjugation of a drug moiety. The amino acid sequence of such molecule is (SEQ ID NO:107):

APELLGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLTCLVK GFYPSDIAVE WESNGQPENN
YKTTPPVLDS DGSFFLYSKL TVDKSRWQQG NVFSCSVMHE
ALHNHYTQKS LCLSPGX

wherein X is a lysine (K) or is absent.

A third preferred IgG1 sequence for the CH2 and CH3 Domains of the Fc Region-containing molecules of the present invention comprises the L234A/L235A substitutions (shown underlined) that reduce or abolish effector function and the S442C substitution (shown underlined) that permits two CH3 domains to be covalently bonded to one another via a disulfide bond or conjugation of a drug moiety. The amino acid sequence of such molecule is (SEQ ID NO:108):

APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLTCLVK GFYPSDIAVE WESNGQPENN
YKTTPPVLDS DGSFFLYSKL TVDKSRWQQG NVFSCSVMHE
ALHNHYTQKS LCLSPGX

wherein X is a lysine (K) or is absent.

The serum half-life of proteins comprising Fc Regions may be increased by increasing the binding affinity of the Fc Region for FcRn. The term “half-life” as used herein means a pharmacokinetic property of a molecule that is a measure of the mean survival time of the molecules following their administration. Half-life can be expressed as the time required to eliminate fifty percent (50%) of a known quantity of the molecule from a subject's (e.g., a human patient or other mammal) body or a specific compartment thereof, for example, as measured in serum, i.e., circulating half-life, or in other tissues. In general, an increase in half-life results in an increase in mean residence time (MRT) in circulation for the administered molecule.

In some embodiments, the ADAM9-binding molecules of the present invention comprise a variant Fc Region that comprises at least one amino acid modification relative to a wild-type Fc Region, such that said molecule has an increased half-life (relative to a molecule comprising a wild-type Fc Region). In some embodiments, the ADAM9-binding molecules of the present invention comprise a variant IgG Fc Region, wherein said variant Fc Region comprises a half-live extending amino acid substitution at one or more positions selected from the group consisting of 238, 250, 252, 254, 256, 257, 256, 265, 272, 286, 288, 303, 305, 307, 308, 309, 311, 312, 317, 340, 356, 360, 362, 376, 378, 380, 382, 413, 424, 428, 433, 434, 435, and 436, wherein the numbering is that of the EU index as in Kabat. Numerous mutations capable of increasing the half-life of an Fc Region-containing molecule are known in the art and include, for example M252Y, S254T, T256E, and combinations thereof. For example, see the mutations described in U.S. Pat. Nos. 6,277,375, 7,083,784; 7,217,797, 8,088,376; U.S. Publication Nos. 2002/0147311; 2007/0148164; and PCT Publication Nos. WO 98/23289; WO 2009/058492; and WO 2010/033279, which are herein incorporated by reference in their entireties. ADAM9-binding molecules with enhanced half-life also include those possessing variant Fc Regions comprising substitutions at two or more of Fc Region residues 250, 252, 254, 256, 257, 288, 307, 308, 309, 311, 378, 428, 433, 434, 435 and 436. In particular, two or more substitutions selected from: T250Q, M252Y, S254T, T256E, K288D, T307Q, V308P, A378V, M428L, N434A, H435K, and Y436I, wherein the numbering is that of the EU index as in Kabat.

In a specific embodiment, an ADAM9-binding molecule of the present invention possesses a variant IgG Fc Region comprising the substitutions:
(A) M252Y, S254T and T256E;
(B) M252Y and S254T;
(C) M252Y and T256E;
(D) T250Q and M428L;
(E) T307Q and N434A;
(F) A378V and N434A;
(G) N434A and Y436I;
(H) V308P and N434A; or
(I) K288D and H435K.

In a preferred embodiment, an ADAM9-binding molecule of the present invention possesses a variant IgG Fc Region comprising any 1, 2, or 3 of the substitutions: M252Y, S254T and T256E. The invention further encompasses ADAM9-binding molecules possessing variant Fc Regions comprising:
(A) one or more mutations which alter effector function and/or FcγR; and
(B) one or more mutations which extend serum half-life.

A fourth preferred IgG1 sequence for the CH2 and CH3 Domains of the Fc Region-containing molecules of the present invention comprises the M252Y, S254T and T256E substitutions (shown underlined), so as to extend the serum half-life. The amino acid sequence of such molecule is (SEQ ID NO:200):

APELLGGPSV FLFPPKPKDT LYITREPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLTCLVK GFYPSDIAVE WESNGQPENN
YKTTPPVLDS DGSFFLYSKL TVDKSRWQQG NVFSCSVMHE
ALHNHYTQKS LSLSPGX

wherein X is a lysine (K) or is absent.

A fifth preferred IgG1 sequence for the CH2 and CH3 Domains of the Fe Region-containing molecules of the present invention comprises the L234A/L235A substitutions (shown underlined) that reduce or abolish effector function and the M252Y, S254T and T256E substitutions (shown underlined), so as to extend the serum half-life. The amino acid sequence of such molecule is (SEQ ID NO:201):

APEAAGGPSV FLFPPKPKDT LYITREPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLTCLVK GFYPSDIAVE WESNGQPENN
YKTTPPVLDS DGSFFLYSKL TVDKSRWQQG NVFSCSVMHE
ALHNHYTQKS LSLSPGX

wherein X is a lysine K) or is absent.

A sixth preferred IgG1 sequence for the CH2 and CH3 Domains of the Fc Region-containing molecules of the present invention comprises the L234A/L235A substitutions (shown underlined) that reduce or abolish effector function and the M252Y, S254T and T256E substitutions (shown underlined), so as to extend the serum half-life and the S442C substitution (shown underlined), so as to permit two CH3 domains to be covalently bonded to one another via a disulfide bond or to permit conjugation of a drug moiety. The amino acid sequence of such molecule is (SEQ ID NO:203):

APEAAGGPSV FLFPPKPKDT LYITREPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLTCLVK GFYPSDIAVE WESNGQPENN
YKTTPPVLDS DGSFFLYSKL TVDKSRWQQG NVFSCSVMHE
ALHNHYTQKS LCLSPGX

wherein X is a lysine (K) or is absent.

For certain antibodies, diabodies and trivalent binding molecules whose Fc Region-containing first and third polypeptide chains are not identical, it is desirable to reduce or prevent homodimerization from occurring between the CH2-CH3 Domains of two first polypeptide chains or between the CH2-CH3 Domains of two third polypeptide chains. The CH2 and/or CH3 Domains of such polypeptide chains need not be identical in sequence, and advantageously are modified to foster complexing between the two polypeptide chains. For example, an amino acid substitution (preferably a substitution with an amino acid comprising a bulky side group forming a “knob”, e.g., tryptophan) can be introduced into the CH2 or CH3 Domain such that steric interference will prevent interaction with a similarly mutated domain and will obligate the mutated domain to pair with a domain into which a complementary, or accommodating mutation has been engineered, i.e., “the hole” (e.g., a substitution with glycine). Such sets of mutations can be engineered into any pair of polypeptides comprising CH2-CH3 Domains that forms an Fc Region to foster heterodimerization. Methods of protein engineering to favor heterodimerization over homodimerization are well known in the art, in particular with respect to the engineering of immunoglobulin-like molecules, and are encompassed herein (see e.g., Ridgway et al. (1996) “‘Knobs-Into-Holes’ Engineering Of Antibody CH3 Domains For Heavy Chain Heterodimerization,” Protein Engr. 9:617-621; Atwell et al. (1997) “Stable Heterodimers From Remodeling The Domain Interface Of A Homodimer Using A Phage Display Library,” J. Mol. Biol. 270: 26-35; and Xie et al. (2005) “A New Format Of Bispecific Antibody: Highly Efficient Heterodimerization, Expression And Tumor Cell Lysis,” J. Immunol. Methods 296:95-101; each of which is hereby incorporated herein by reference in its entirety).

A preferred knob is created by modifying an IgG Fc Region to contain the modification T366W. A preferred hole is created by modifying an IgG Fc Region to contain the modification T366S, L368A and Y407V. To aid in purifying the hole-bearing third polypeptide chain homodimer from the final bispecific heterodimeric Fc Region-containing molecule, the protein A binding site of the hole-bearing CH2 and CH3 Domains of the third polypeptide chain is preferably mutated by amino acid substitution at position 435 (H435R). Thus, the hole-bearing third polypeptide chain homodimer will not bind to protein A, whereas the bispecific heterodimer will retain its ability to bind protein A via the protein A binding site on the first polypeptide chain. In an alternative embodiment, the hole-bearing third polypeptide chain may incorporate amino acid substitutions at positions 434 and 435 (N434A/N435K).

A preferred IgG amino acid sequence for the CH2 and CH3 Domains of the first polypeptide chain of an Fc Region-containing molecule of the present invention will have the “knob-bearing” sequence (SEQ ID NO:109):

APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLWCLVK GFYPSDIAVE WESNGQPENN
YKTTPPVLDS DGSFFLYSKL TVDKSRWQQG NVFSCSVMHE
ALHNHYTQKS LSLSPGX

wherein X is a lysine (K) or is absent.

A preferred IgG amino acid sequence for the CH2 and CH3 Domains of the second polypeptide chain of an Fc Region-containing molecule of the present invention having two polypeptide chains (or the third polypeptide chain of an Fc Region-containing molecule having three, four, or five polypeptide chains) will have the “hole-bearing” sequence (SEQ ID NO:110):

APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLSCAVK GFYPSDIAVE WESNGQPENN
YKTTPPVLDS DGSFFLVSKL TVDKSRWQQG NVFSCSVMHE
ALHNRYTQKS LSLSPGX

wherein X is a lysine K) or is absent.

As will be noted, the CH2-CH3 Domains of SEQ ID NO:109 and SEQ ID NO:110 include a substitution at position 234 with alanine and 235 with alanine, and thus form an Fc Region exhibit decreased (or substantially no) binding to FcγRIA (CD64), FcγRIIA (CD32A), FcγRIIB (CD32B), FcγRIIIA (CD16a) or FcγRIIIB (CD16b) (relative to the binding exhibited by the wild-type Fc Region (SEQ ID NO:1). The invention also encompasses such CH2-CH3 Domains, which comprise the wild-type alanine residues, alternative and/or additional substitutions which modify effector function and/or FYR binding activity of the Fc region.

The invention also encompasses such CH2-CH3 Domains, which further comprise one or more half-live extending amino acid substitutions. In particular, the invention encompasses such hole-bearing and such knob-bearing CH2-CH3 Domains which further comprise the M252Y/S254T/T256E substitutions.

An exemplary knob-bearing CH2 and CH3 Domains comprising the L234A and L235A substitutions and further comprising the M252Y, S254T, and T256E substitutions is provided below (SEQ ID NO:204):

APEAAGGPSV FLFPPKPKDT LYITREPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLWCLVK GFYPSDIAVE WESNGQPENN
YKTTPPVLDS DGSFFLYSKL TVDKSRWQQG NVFSCSVMHE
ALHNHYTQKS LSLSPGX

wherein X is a lysine (K) or is absent.

An exemplary hole-bearing CH2 and CH3 Domains comprising the L234A and L235A substitutions and further comprising the M252Y, S254T, and T256E substitutions is provided below (SEQ ID NO:205):

APEAAGGPSV FLFPPKPKDT LYITREPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLSCAVK GFYPSDIAVE WESNGQPENN
YKTTPPVLDS DGSFFLVSKL TVDKSRWQQG NVFSCSVMHE
ALHNRYTQKS LSLSPGX

wherein X is a lysine (K) or is absent.

It is preferred that the first polypeptide chain will have a “knob-bearing” CH2-CH3 sequence, such as that of SEQ ID NO:109. However, as will be recognized, a “hole-bearing” CH2-CH3 Domain (e.g., SEQ ID NO:110 could be employed in the first polypeptide chain, in which case, a “knob-bearing” CH2-CH3 Domain (e.g., SEQ ID NO:109) would be employed in the second polypeptide chain of an Fc Region-containing molecule of the present invention having two polypeptide chains (or in the third polypeptide chain of an Fc Region-containing molecule having three, four, or five polypeptide chains).

In other embodiments, the invention encompasses ADAM9-binding molecules comprising CH2 and/or CH3 Domains that have been engineered to favor heterodimerization over homodimerization using mutations known in the art, such as those disclosed in PCT Publication Nos. WO 2007/110205; WO 2011/143545; WO 2012/058768; and WO 2013/06867, all of which are incorporated herein by reference in their entirety.

### X. Effector Cell Binding Capabilities

As provided herein, the ADAM9-binding molecules of the invention can be engineered to comprise a combination of epitope-binding sites that recognize a set of antigens unique to a target cell or tissue type. In particular, the present invention relates to multispecific ADAM9-binding molecules that are capable of binding to an epitope of ADAM9 and an epitope of a molecule present on the surface of an effector cell, such as a T lymphocyte, a natural killer (NK) cell or other mononuclear cell. For example, the ADAM9-binding molecules of the present invention may be construction to comprise an epitope-binding site that immunospecifically binds CD2, CD3, CD8, CD16, T-Cell Receptor (TCR), or NKG2D. The invention also relates to trispecific ADAM9-binding molecules that are capable of binding to an epitope of CD3 and an epitope of CD8 (see, e.g., PCT Publication No. WO 2015/026894).

### A. CD2 Binding Capabilities

In one embodiment, the bispecific, trispecific or multispecific ADAM9-binding molecules of the invention are capable of binding to an epitope of ADAM9 and an epitope of CD2. CD2 is a cell adhesion molecule found on the surface of T-cells and natural killer (NK) cells. CD2 enhances NK cell cytotoxicity, possibly as a promoter of NK cell nanotube formation (Mace, E. M. et al. (2014) “Cell Biological Steps and Checkpoints in Accessing NK Cell Cytotoxicity,” Immunol. Cell. Biol. 92(3):245-255; Comerci, C. J. et al. (2012) “CD2 Promotes Human Natural Killer Cell Membrane Nanotube Formation,” PLoS One 7(10):e47664:1-12). Molecules that specifically bind CD2 include the anti-CD2 antibody “Lo-CD2a.”

The amino acid sequence of the VH Domain of Lo-CD2a (ATCC Accession No: 11423); SEQ ID NO:111) is shown below (CDR_{H} residues are shown underlined):

EVQLQQSGPE LQRPGASVKL SCKASGYIFT EYYMYWVKQR
PKQGLELVGR IDPEDGSIDY VEKFKKKATL TADTSSNTAY
MQLSSLTSED TATYFCARGK FNYRFAYWGQ GTLVTVSS

The amino acid sequence of the VL Domain of Lo-CD2a (ATCC Accession No: 11423; SEQ ID NO:112) is shown below CDR_{L} residues are shown underlined):

DVVLTQTPPT LLATIGQSVS ISCRSSQSLL HSSGNTYLNW
LLQRTGQSPQ PLIYLVSKLE SGVPNRFSGS GSGTDFTLKI
SGVEAEDLGV YYCMQFTHYP YTFGAGTKLE LK

### B. CD3 Binding Capabilities

In one embodiment, the bispecific, trispecific or multispecific ADAM9-binding molecules of the invention are capable of binding to an epitope of ADAM9 and an epitope of CD3. CD3 is a T-cell co-receptor composed of four distinct chains (Wucherpfennig, K. W. et al. (2010) “Structural Biology Of The T-Cell Receptor: Insights Into Receptor Assembly, Ligand Recognition, And Initiation Of Signaling,” Cold Spring Harb. Perspect. Biol. 2(4):a005140; pages 1-14). In mammals, the complex contains a CD3γ chain, a CD3δ chain, and two CD3ε chains. These chains associate with a molecule known as the T-Cell Receptor (TCR) in order to generate an activation signal in T lymphocytes. In the absence of CD3, TCRs do not assemble properly and are degraded (Thomas, S. et al. (2010) “Molecular Immunology Lessons From Therapeutic T-Cell Receptor Gene Transfer,” Immunology 129(2):170-177). CD3 is found bound to the membranes of all mature T-cells, and in virtually no other cell type (see, Janeway, C. A. et al. (2005) In: IMMUNOBIOLOGY: THE IMMUNE SYSTEM IN HEALTH AND DISEASE,” 6th ed. Garland Science Publishing, NY, pp. 214-216; Sun, Z. J. et al. (2001) “Mechanisms Contributing To T Cell Receptor Signaling And Assembly Revealed By The Solution Structure Of An Ectodomain Fragment Of The CD3ε:γ Heterodimer,” Cell 105(7):913-923; Kuhns, M. S. et al. (2006) “Deconstructing The Form And Function Of The TCR/CD3 Complex,” Immunity. 2006 February; 24(2):133-139). Molecules that specifically binds CD3 include the anti-CD3 antibodies “CD3 mAb-1” and “OKT3.” The anti-CD3 antibody CD3 mAb-1 is capable of binding non-human primates (e.g., cynomolgus monkey).

The amino acid sequence of the VH Domain of CD3 mAb-1 VH(1) (SEQ ID NO:113) is shown below (CDR_{H} residues are shown underlined):

EVQLVESGGG LVQPGGSLRL SCAASGFTFS TYAMNWVRQA
PGKGLEWVGR IRSKYNNYAT YYADSVKDRF TISRDDSKNS
LYLQMNSLKT EDTAVYYCVR HGNFGNSYVS WFAYWGQGTL
VTVSS

The amino acid sequence of an alternative VH Domain of CD3 mAb-1 VH(2) (SEQ ID NO:114) is shown below (CDR_{H} residues are shown in single underline; differences relative to the VH Domain of CD3 mAb-1 VH(1) (SEQ ID NO:92) are shown in double underline).

EVQLVESGGG LVQPGGSLRL [symbol] TYAMNWVRQA
PGKGLEWV [symbol] IRSKYNNYAT YYADSVKDRF TISRDDSKNS
LYLQMNSLKT EDTAVYYCVR HGNFGNSYVS WFAYWGQGTL
VTVSS

The amino acid sequence of the VL Domain of CD3 mAb-1 (SEQ ID NO:115) is shown below (CDR_{L} residues are shown underlined):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG

The VH Domain of CD3 mAb-1 VH(1) (SEQ ID NO:) may be used with the VL Domain of CD3 mAb-1 (SEQ ID NO:) to form a functional CD3-binding molecule in accordance with the present invention. Likewise, the VH Domain of CD3 mAb-1 VH(2) (SEQ ID NO:) may be used with the VL Domain of CD3 mAb-1 (SEQ ID NO:) to form a functional CD3-binding molecule in accordance with the present invention.

As discussed below, in order to illustrate the present invention, bispecific ADAM9×CD3-binding molecules were produced. In some of the ADAM9×CD3 constructs, a variant of CD3 mAb-1 was employed. The variant “CD3 mAb-1 (D65G),” comprises the VL Domain of CD3 mAb-1 (SEQ ID NO:115) and a VH CD3 mAb-1 Domain having a D65G substitution (Kabat position 65, corresponding to residue 68 of SEQ ID NO:113).

The amino acid sequence of the VH Domain of CD3 mAb-1 (D65G) (SEQ ID NO:116) is shown below (CDR_{H} residues are shown underlined, the substituted position (D65G) is shown in double underline):

EVQLVESGGG LVQPGGSLRL SCAASGFTFS TYAMNWVRQA
PGKGLEWVGR IRSKYNNYAT [symbol] TISRDDSKNS
LYLQMNSLKT EDTAVYYCVR HGNFGNSYVS WFAYWGQGTL
VTVSS

Alternatively, an affinity variant of CD3 mAb-1 may be incorporated. Variants include a low affinity variant designated “CD3 mAb-1 Low” and a variant having a faster off rate designated “CD3 mAb-1 Fast.” The VL Domain of CD mAb1 (SEQ ID NO:115) is common to CD3 mAb-1 Low and CD3 mAb1 Fast and is provided above. The amino acid sequences of the VH Domains of each of CD3 mAb-1 Low and CD3 mAb-1 Fast are provided below.

The amino acid sequence of the VH Domain of anti-human CD3 mAb-1 Low (SEQ ID NO:117) is shown below (CDR_{H} residues are shown underlined; differences relative to the VH Domain of CD3 mAb-1 VH(1) (SEQ ID NO:113) are shown in double underline):

EVQLVESGGG LVQPGGSLRL SCAASGFTFS TYAMNWVRQA
PGKGLEWVGR IRSKYNNYAT [symbol] TISRDDSKNS
LYLQMNSLKT EDTAVYYCVR [symbol] WFAYWGQGTL
VTVSS

The amino acid sequence of the VH Domain of anti-human CD3 mAb-1 Fast (SEQ ID NO:118) is shown below (CDR_{H} residues are shown underlined; differences relative to the VH Domain of CD3 mAb-1 VH(1) (SEQ ID NO:113) are shown in double underline):

EVQLVESGGG LVQPGGSLRL SCAASGFTFS TYAMNWVRQA
PGKGLEWVGR IRSKYNNYAT YYADSVKGRF TISRDDSKNS
LYLQMNSLKT EDTAVYYCVR [symbol] WFAYWGQGTL
VTVSS

Another anti-CD3 antibody, which may be utilized is antibody Muromonab-CD3 “OKT3” (Xu et al. (2000) “In Vitro Characterization Of Five Humanized OKT3 Effector Function Variant Antibodies,” Cell. Immunol. 200:16-26; Norman, D. J. (1995) “Mechanisms Of Action And Overview Of OKT3,” Ther. Drug Monit. 17(6):615-620; Canafax, D. M. et al. (1987) “Monoclonal Antilymphocyte Antibody (OKT3) Treatment Of Acute Renal Allograft Rejection,” Pharmacotherapy 7(4):121-124; Swinnen, L. J. et al. (1993) “OKT3 Monoclonal Antibodies Induce Interleukin-6 And Interleukin-10: A Possible Cause Of Lymphoproliferative Disorders Associated With Transplantation,” Curr. Opin. Nephrol. Hypertens. 2(4):670-678).

The amino acid sequence of the VH Domain of OKT3 (SEQ ID NO:119) is shown below (CDR_{H} residues are shown underlined):

QVQLQQSGAE LARPGASVKM SCKASGYTFT RYTMHWVKQR
PGQGLEWIGY INPSRGYTNY NQKFKDKATL TTDKSSSTAY
MQLSSLTSED SAVYYCARYY DDHYCLDYWG QGTTLTVSS

The amino acid sequence of the VL Domain of OKT3 (SEQ ID NO:120) is shown below (CDR_{L} residues are shown underlined):

QIVLTQSPAI MSASPGEKVT MTCSASSSVS YMNWYQQKSG
TSPKRWIYDT SKLASGVPAH FRGSGSGTSY SLTISGMEAE
DAATYYCQQW SSNPFTFGSG TKLEINR

Additional anti-CD3 antibodies that may be utilized include but are not limited to those described in PCT Publication Nos. WO 2008/119566; and WO 2005/118635.

### C. CD8 Binding Capabilities

In one embodiment, the bispecific, trispecific or multispecific ADAM9-binding molecules of the invention are capable of binding to an epitope of ADAM9 and an epitope of CD8. CD8 is a T-cell co-receptor composed of two distinct chains (Leahy, D. J., (1995) “A Structural View of CD4 and CD8,” FASEB J., 9:17-25) that is expressed on Cytotoxic T-cells. The activation of CD8⁺ T-cells has been found to be mediated through co-stimulatory interactions between an antigen:major histocompability class I (MHC I) molecule complex that is arrayed on the surface of a target cell and a complex of CD8 and the T-cell Receptor, that are arrayed on surface of the CD8⁺ T-cell (Gao, G., and Jakobsen, B., (2000). “Molecular interactions of coreceptor CD8 and MHC class I: the molecular basis for functional coordination with the T-Cell Receptor”. Immunol Today 21: 630-636). Unlike MHC II molecules, which are expressed by only certain immune system cells, MHC I molecules are very widely expressed. Thus, cytotoxic T-cells are capable of binding to a wide variety of cell types. Activated cytotoxic T-cells mediate cell killing through their release of the cytotoxins perforin, granzymes, and granulysin. Antibodies that specifically bind CD8 include the anti-CD8 antibodies “OKT8” and “TRX2.”

The amino acid sequence of the VH Domain of OKT8 (SEQ ID NO:121) is shown below (CDR_{H} residues are shown underlined):

QVQLLESGPE LLKPGASVKM SCKASGYTFT DYNMHWVKQS
HGKSLEWIGY IYPYTGGTGY NQKFKNKATL TVDSSSSTAY
MELRSLTSED SAVYYCARNF RYTYWYFDVW GQGTTVTVSS

The amino acid sequence of the VL Domain of OKT8 (SEQ ID NO:122) is shown below (CDR_{L} residues are shown underlined):

DIVMTQSPAS LAVSLGQRAT ISCRASESVD SYDNSLMHWY
QQKPGQPPKV LIYLASNLES GVPARFSGSG SRTDFTLTID
PVEADDAATY YCQQNNEDPY TFGGGTKLEI KR

The amino acid sequence of the VH Domain of TRX2 (SEQ ID NO:123) is shown below (CDR_{H} residues are shown underlined):

QVQLVESGGG VVQPGRSLRL SCAASGFTFS DFGMNWVRQA
PGKGLEWVAL IYYDGSNKFY ADSVKGRFTI SRDNSKNTLY
LQMNSLRAED TAVYYCAKPH YDGYYHFFDS WGQGTLVTVS
S

The amino acid sequence of the VL Domain of TRX2 (SEQ ID NO:124) is shown below (CDR_{L} residues are shown underlined):

DIQMTQSPSS LSASVGDRVT ITCKGSQDIN NYLAWYQQKP
GKAPKLLIYN TDILHTGVPS RFSGSGSGTD FTFTISSLQP
EDIATYYCYQ YNNGYTFGQG TKVEIK

### D. CD16 Binding Capabilities

In one embodiment, multispecific ADAM9-binding molecules of the invention are capable of binding to an epitope of ADAM9 and an epitope of CD16. CD16 is the FcγRIIIA receptor. CD16 is expressed by neutrophils, eosinophils, natural killer (NK) cells, and tissue macrophages that bind aggregated but not monomeric human IgG (Peltz, G. A. et al. (1989) “Human Fc Gamma RIII: Cloning, Expression, And Identification Of The Chromosomal Locus Of Two Fc Receptors For IgG,” Proc. Natl. Acad. Sci. (U.S.A.) 86(3):1013-1017; Bachanova, V. et al. (2014) “NK Cells In Therapy Of Cancer,” Crit. Rev. Oncog. 19(1-2):133-141; Miller, J. S. (2013) “Therapeutic Applications: Natural Killer Cells In The Clinic,” Hematology Am. Soc. Hematol. Educ. Program. 2013:247-253; Youinou, P. et al. (2002) “Pathogenic Effects Of Anti-Fc Gamma Receptor IIIB (CD16) On Polymorphonuclear Neutrophils In Non-Organ-Specific Autoimmune Diseases,” Autoimmun Rev. 1(1-2):13-19; Peipp, M. et al. (2002) “Bispecific Antibodies Targeting Cancer Cells,” Biochem. Soc. Trans. 30(4):507-511). Molecules that specifically bind CD16 include the anti-CD16 antibodies “3G8” and “A9.” Humanized A9 antibodies are described in PCT Publication No. WO 03/101485.

The amino acid sequence of the VH Domain of 3G8 (SEQ ID NO:125) is shown below (CDR_{H} residues are shown underlined):

QVTLKESGPG ILQPSQTLSL TCSFSGFSLR TSGMGVGWIR
QPSGKGLEWL AHIWWDDDKR YNPALKSRLT ISKDTSSNQV
FLKIASVDTA DTATYYCAQI NPAWFAYWGQ GTLVTVSA

The amino acid sequence of the VL Domain of 3G8 (SEQ ID NO:126) is shown below (CDR_{L} residues are shown underlined):

DTVLTQSPAS LAVSLGQRAT ISCKASQSVD FDGDSFMNWY
QQKPGQPPKL LIYTTSNLES GIPARFSASG SGTDFTLNIH
PVEEEDTATY YCQQSNEDPY TFGGGTKLEI K

The amino acid sequence of the VH Domain of A9 (SEQ ID NO:127) is shown below (CDR_{H} residues are shown underlined):

QVQLQQSGAE LVRPGTSVKI SCKASGYTFT NYWLGWVKQR
PGHGLEWIGD IYPGGGYTNY NEKFKGKATV TADTSSRTAY
VQVRSLTSED SAVYFCARSA SWYFDVWGAR TTVTVSS

The amino acid sequence of the VL Domain of A9 (SEQ ID NO:128) is shown below (CDR_{L} residues are shown underlined):

DIQAVVTQES ALTTSPGETV TLTCRSNTGT VTTSNYANWV
QEKPDHLFTG LIGHTNNRAP GVPARFSGSL IGDKAALTIT
GAQTEDEAIY FCALWYNNHW VFGGGTKLTVL

Additional anti-CD16 antibodies that may be utilized include but are not limited to those described in PCT Publication Nos. WO 03/101485; and WO 2006/125668.

### E. TCR Binding Capabilities

In one embodiment, the bispecific, trispecific or multispecific ADAM9-binding molecules of the invention are capable of binding to an epitope of ADAM9 and an epitope of the T Cell Receptor (TCR). The T Cell Receptor is natively expressed by CD4⁺ or CD8⁺ T cells, and permits such cells to recognize antigenic peptides that are bound and presented by class I or class II MHC proteins of antigen-presenting cells. Recognition of a pMHC (peptide-MHC) complex by a TCR initiates the propagation of a cellular immune response that leads to the production of cytokines and the lysis of the antigen-presenting cell (see, e.g., Armstrong, K. M. et al. (2008) “Conformational Changes And Flexibility In T-Cell Receptor Recognition Of Peptide-MHC Complexes,” Biochem. J. 415(Pt 2):183-196; Willemsen, R. (2008) “Selection Of Human Antibody Fragments Directed Against Tumor T-Cell Epitopes For Adoptive T-Cell Therapy,” Cytometry A. 73(11):1093-1099; Beier, K. C. et al. (2007) “Master Switches Of T-Cell Activation And Differentiation,” Eur. Respir. J. 29:804-812; Mallone, R. et al. (2005) “Targeting T Lymphocytes For Immune Monitoring And Intervention In Autoimmune Diabetes,” Am. J. Ther. 12(6):534-550). CD3 is the receptor that binds to the TCR (Thomas, S. et al. (2010) “Molecular Immunology Lessons From Therapeutic T-Cell Receptor Gene Transfer,” Immunology 129(2):170-177; Guy, C. S. et al. (2009) “Organization Of Proximal Signal Initiation At The TCR:CD3 Complex,” Immunol. Rev. 232(1):7-21; St. Clair, E. W. (Epub 2009 Oct. 12) “Novel Targeted Therapies ForAutoimmunity,” Curr. Opin. Immunol. 21(6):648-657; Baeuerle, P. A. et al. (Epub 2009 Jun. 9) “Bispecific T-Cell Engaging Antibodies For Cancer Therapy,” Cancer Res. 69(12):4941-4944; Smith-Garvin, J. E. et al. (2009) “T Cell Activation,” Annu. Rev. Immunol. 27:591-619; Renders, L. et al. (2003) “Engineered CD3 Antibodies For Immunosuppression,” Clin. Exp. Immunol. 133(3):307-309).

Molecules that specifically bind to the T Cell Receptor include the anti-TCR antibody “BMA 031” (EP 0403156; Kurrle, R. et al. (1989) “BMA 031—A TCR-Specific Monoclonal Antibody For Clinical Application,” Transplant Proc. 21(1 Pt 1):1017-1019; Nashan, B. et al. (1987) “Fine Specificity Of A Panel Of Antibodies Against The TCR/CD3 Complex,” Transplant Proc. 19(5):4270-4272; Shearman, C. W. et al. (1991) “Construction, Expression, And Biologic Activity Of Murine/Human Chimeric Antibodies With Specificity For The Human α/β T Cell,” J. Immunol. 146(3):928-935; Shearman, C. W. et al. (1991) “Construction, Expression And Characterization of Humanized Antibodies Directed Against The Human α/β T Cell Receptor,” J. Immunol. 147(12):4366-4373; and PCT Publication No. WO 2010/027797).

The amino acid sequence of a VH Domain of BMA 031 (SEQ ID NO:129) is shown below (CDR_{H} residues are shown underlined):

QVQLVQSGAE VKKPGASVKV SCKASGYKFT SYVMHWVRQA
PGQGLEWIGY INPYNDVTKY NEKFKGRVTI TADKSTSTAY
LQMNSLRSED TAVHYCARGS YYDYDGFVYW GQGTLVTVSS

The amino acid sequence of the VL Domain of BMA 031 (SEQ ID NO:130) is shown below (CDR_{L} residues are shown underlined):

EIVLTQSPAT LSLSPGERAT LSCSATSSVS YMHWYQQKPG
KAPKRWIYDT SKLASGVPSR FSGSGSGTEF TLTISSLQPE
DFATYYCQQW SSNPLTFGQG TKLEIK

### F. NKG2D Binding Capabilities

In one embodiment, multispecific ADAM9-binding molecules of the invention are capable of binding to an epitope of ADAM9 and an epitope of the NKG2D receptor. The NKG2D receptor is expressed on all human (and other mammalian) Natural Killer cells (Bauer, S. et al. (1999) “Activation Of NK Cells And T Cells By NKG2D, A Receptor For Stress-Inducible MICA,” Science 285(5428):727-729; Jamieson, A. M. et al. (2002) “The Role Of The NKG2D Immunoreceptor In Immune Cell Activation And Natural Killing,” Immunity 17(1):19-29) as well as on all CD8⁺ T cells (Groh, V. et al. (2001) “Costimulation Of CD8αβ T Cells By NKG2D Via Engagement By MIC Induced On Virus-Infected Cells,” Nat. Immunol. 2(3):255-260; Jamieson, A. M. et al. (2002) “The Role Of The NKG2D Immunoreceptor In Immune Cell Activation And Natural Killing,” Immunity 17(1):19-29). Such binding ligands, and particularly those which are not expressed on normal cells, include the histocompatibility 60 (H60) molecule, the product of the retinoic acid early inducible gene-1 (RAE-1), and the murine UL16-binding proteinlike transcript 1 (MULT1) (Raulet D. H. (2003) “Roles Of The NKG2D ImmunoreceptorAnd Its Ligands,” Nature Rev. Immunol. 3:781-790; Coudert, J. D. et al. (2005) “Altered NKG2D Function In NK Cells Induced By Chronic Exposure To Altered NKG2D Ligand-Expressing Tumor Cells,” Blood 106:1711-1717). Molecules that specifically bind to the NKG2D Receptor include the anti-NKG2D antibodies “KYK-1.0” and “KYK-2.0” (Kwong, K Y et al. (2008) “Generation, Affinity Maturation, And Characterization Of A Human Anti-Human NKG2D Monoclonal Antibody With Dual Antagonistic And Agonistic Activity,” J. Mol. Biol. 384:1143-1156).

The amino acid sequence of the VH Domain of KYK-1.0 (SEQ ID NO:131) is shown below (CDR_{H} residues are shown underlined):

EVQLVESGGG VVQPGGSLRL SCAASGFTFS SYGMHWVRQA
PGKGLEWVAF IRYDGSNKYY ADSVKGRFTI SRDNSKNTKY
LQMNSLRAED TAVYYCAKDR FGYYLDYWGQ GTLVTVSS

The amino acid sequence of the VL Domain of KYK-1.0 (SEQ ID NO:132) is shown below (CDR_{L} residues are shown underlined):

QPVLTQPSSV SVAPGETARI PCGGDDIETK SVHWYQQKPG
QAPVLVIYDD DDRPSGIPER FFGSNSGNTA TLSISRVEAG
DEADYYCQVW DDNNDEWVFG GGTQLTVL

The amino acid sequence of a VH Domain of KYK-2.0 (SEQ ID NO:133) is shown below (CDR_{H} residues are shown underlined):

QVQLVESGGG LVKPGGSLRL SCAASGFTFS SYGMHWVRQA
PGKGLEWVAF IRYDGSNKYY ADSVKGRFTI SRDNSKNTLY
LQMNSLRAED TAVYYCAKDR GLGDGTYFDY WGQGTTVTVS S

The amino acid sequence of a VL Domain of KYK-2.0 (SEQ ID NO:134) is shown below (CDR_{L} residues are shown underlined):

QSALTQPASV SGSPGQSITI SCSGSSSNIG NNAVNWYQQL
PGKAPKLLIY YDDLLPSGVS DRFSGSKSGT SAFLAISGLQ
SEDEADYYCA AWDDSLNGPV FGGGTKLTVL

### XI. Multispecific ADAM9-Binding Molecules

### A. ADAM9×CD3 Bispecific Two Chain Diabodies

The VL and VH Domains of the above-described optimized humanized anti-ADAM9 MAB-A antibody is used to construct ADAM9×CD3 bispecific diabodies composed of two covalently linked polypeptide chains and comprising the above-discussed optimized humanized VL and VH Domains of MAB-A. The general structure and amino acid sequences of such ADAM9×CD3 bispecific diabodies is provided below.

The first polypeptide chain of one exemplary ADAM9×CD3 bispecific two chain diabody comprises, in the N-terminal to C-terminal direction: an N-terminus; the VL Domain of an anti-ADAM9 antibody (e.g., hMAB-A VL (2) (SEQ ID NO:55); an intervening spacer peptide (Linker 1: GGGSGGGG (SEQ ID NO:69)); the VH Domain of an anti-CD3 antibody (e.g., CD3 mAb 1 (D65G) (SEQ ID NO:116)); a cysteine-containing intervening spacer peptide (Linker 2: GGCGGG (SEQ ID NO:70)); a Heterodimer-Promoting (e.g., an E-coil) Domain (EVAALEK-EVAALEK-EVAALEK-EVAALEK (SEQ ID NO:82)); and a C-terminus.

The second polypeptide chain of such an exemplary ADAM9×CD3 bispecific two chain diabody comprises, in the N-terminal to C-terminal direction: an N-terminus; the VL Domain of a corresponding anti-CD3 antibody (e.g., a VL domain that in association with the VH Domain of the first polypeptide chain forms a CD3-binding site, e.g., the VL Domain of CD3 mAb-1 (SEQ ID NO:115); an intervening spacer peptide (Linker 1: GGGSGGGG (SEQ ID NO:69)); the VH Domain of a corresponding anti-ADAM9 antibody (e.g., a VH domain that in association with the VL Domain of the first polypeptide chain forms an ADAM9-binding site, e.g., hMAB-A VH (2) (SEQ ID NO:17); a cysteine-containing intervening spacer peptide (Linker 2: GGCGGG (SEQ ID NO:70)); a Heterodimer-Promoting (e.g., K-coil) Domain (KVAALKE-KVAALKE-KVAALKE-KVAALKE (SEQ ID NO:83)); and a C-terminus.

As provided herein, alternative linkers and/or alternative Heterodimer-Promoting Domains may be utilized in the generation of such diabodies. For example, the first polypeptide chain of an alternative exemplary ADAM9×CD3 bispecific two chain diabody may comprise, in the N-terminal to C-terminal direction: an N-terminus; the VL Domain of an anti-ADAM9 antibody; the intervening spacer peptide (Linker 1: GGGSGGGG (SEQ ID NO:69)); the VH Domain of the anti-CD3 antibody or of a corresponding anti-CD3 antibody; an intervening spacer peptide (Linker 2: ASTKG (SEQ ID NO:74)); a cysteine-containing Heterodimer-Promoting (e.g., K-coil) Domain (KVAACKE-KVAALKE-KVAALKE-KVAALKE (SEQ ID NO:85)); and a C-terminus. The second polypeptide chain of such alternative exemplary diabody may comprise, in the N-terminal to C-terminal direction: an N-terminus; the VL Domain of a corresponding anti-CD3 antibody; an intervening spacer peptide (Linker 1: GGGSGGGG (SEQ ID NO:69)); the VH Domain of a corresponding anti-ADAM9 antibody; an intervening spacer peptide (Linker 2: ASTKG (SEQ ID NO:74)); a cysteine-containing Heterodimer-Promoting (e.g., E-coil) Domain (EVAACEK-EVAALEK-EVAALEK-EVAALEK (SEQ ID NO:84)); and a C-terminus.

A representative ADAM9×CD3 bispecific two chain diabody (“DART-1”) comprising the VH and VL Domains of hMAB-A (2.2) and the VH and VL Domains of a CD3 mAb-1 is constructed.

The amino acid sequence of the first polypeptide chain of DART-1 (SEQ ID NO:135) is shown below (the sequence of the hMAb-A VL(2) Domain (SEQ ID NO:55) is underlined; the sequence of the CD3 mAb-1 (D65G) VH Domain (SEQ ID NO:116) is italicised):

DIVMTQSPDS LAVSLGERAT ISCKASQSVD YSGDSYMNWY
QQKPGQPPKL LIYAASDLES GIPARFSGSG SGTDFTLTIS
SLEPEDFATY YCQQSHEDPF TFGQGTKLEI KGGGSGGGGE
VQLVESGGGL VQPGGSLRLS CAASGFTFST YAMNWVRQAP
GKGLEWVGRI RSKYNNYATY YADSVKGRFT ISRDDSKNSL
YLQMNSLKTE DTAVYYCVRH GNFGNSYVSW FAYWGQGTLV
TVSSGGCGGG EVAALEKEVA ALEKEVAALE KEVAALEK

The amino acid sequence of the second polypeptide chain of DART-1 (SEQ ID NO:136) is shown below (the sequence of the hMAB-A VH (2) Domain (SEQ ID NO:17) is underlined; the sequence of the CD3 mAb-1 VL Domain (SEQ ID NO:115) is italicised):

QAVVTQEPSL TVSPGGTVTL TCRSSTGAVT TSNYAMWVQQ
KPGQAPRGLI GGTNKRAPWT PARFSGSLLG GKAALTITGA
QAEDEADYYC ALWYSNLWVF GGGTKLTVLG GGGSGGGGEV
QLVESGGGLV KPGGSLRLSC AASGFTFSSY WMHWVRQAPG
KGLEWVGEII PIFGHTNYNE KFKSRFTISL DNSKNTLYLQ
MGSLRAEDTA VYYCARGGYY YYGSRDYFDY WGQGTTVTVS
SGGCGGGKVA ALKEKVAALK EKVAALKEKV AALKE

### B. ADAM9×CD3 Bispecific Three Chain Diabodies

An ADAM9×CD3 diabody having three chains and possessing an Fc Region is generated having one binding site specific for ADAM9 (comprising humanized/optimized VH and VL Domains of hMAB-A) and one binding site specific for CD3 (comprising the VL and VH Domains of CD3 mAb 1 (D65G)). The diabody is designated “DART-2.”

The first polypeptide chain of the exemplary ADAM9×CD3 bispecific three chain diabodies comprises, in the N-terminal to C-terminal direction: an N-terminus; the VL Domain of an anti-ADAM9 antibody (e.g., hMAB-A VL (2) (SEQ ID NO:55)); an intervening spacer peptide (Linker 1: GGGSGGGG (SEQ ID NO:69)); the VH Domain of CD3 mAb 1 (D65G) (SEQ ID NO:116); an intervening spacer peptide (Linker 2: ASTKG (SEQ ID NO:74)); a cysteine-containing Heterodimer-Promoting (E-coil) Domain (EVAACEK-EVAALEK-EVAALEK-EVAALEK (SEQ ID NO:82)); an intervening spacer peptide (Linker 3: GGGDKTHTCPPCP (SEQ ID NO:94)); a knob-bearing IgG1 CH2-CH3 Domain (SEQ ID NO:109); and a C-terminus. Polynucleotides encoding this polypeptide chain may encode the C-terminal lysine residue of SEQ ID NO:109 (i.e., X of SEQ ID NO:109), however, as discussed above, this lysine residue may be post-translationally removed in some expression systems. Accordingly, the invention encompasses such a first polypeptide chain that contains such lysine residue (i.e., SEQ ID NO:109, wherein X is lysine), as well as a first polypeptide chain that lacks such lysine residue (i.e., SEQ ID NO:109, wherein X is absent). The amino acid sequences of such first polypeptide chain of DART-2 (SEQ ID NO:137) is provided below (the sequence of the hMAB-A VL (2) Domain (SEQ ID NO:55) is underlined; the sequence of the CD3 mAb-1 (D65G) VH Domain (SEQ ID NO:116) is italicised):

DIVMTQSPDS LAVSLGERAT ISCKASQSVD YSGDSYMNWY
QQKPGQPPKL LIYAASDLES GIPARFSGSG SGTDFTLTIS
SLEPEDFATY YCQQSHEDPF TFGQGTKLEI KGGGSGGGGE
VQLVESGGGL VQPGGSLRLS CAASGFTFST YAMNWVRQAP
GKGLEWVGRI RSKYNNYATY YADSVKGRFT ISRDDSKNSL
YLQMNSLKTE DTAVYYCVRH GNFGNSYVSW FAYWGQGTLV
TVSSASTKGE VAACEKEVAA LEKEVAALEK EVAALEKGGG
DKTHTCPPCP APEAAGGPSV FLFPPKPKDT LMISRTPEVT
CVVVDVSHED PEVKFNWYVD GVEVHNAKTK PREEQYNSTY
RVVSVLTVLH QDWLNGKEYK CKVSNKALPA PIEKTISKAK
GQPREPQVYT LPPSREEMTK NQVSLWCLVK GFYPSDIAVE
WESNGQPENN YKTTPPVLDS DGSFFLYSKL TVDKSRWQQG
NVFSCSVMHE ALHNHYTQKS LSLSPGX

wherein X is Lysine (K) or is absent.

The second polypeptide chain of the exemplary ADAM9×CD3 bispecific three chain diabodies comprises, in the N-terminal to C-terminal direction: an N-terminus; the VL Domain of CD3 mAb 1 (SEQ ID NO:115); an intervening spacer peptide (Linker 1: GGGSGGGG (SEQ ID NO:69)); the VH Domain of an anti-ADAM9 antibody (e.g., hMAB-A VH (2) (SEQ ID NO:17)); an intervening spacer peptide (Linker 2: ASTKG (SEQ ID NO:74)); a cysteine-containing Heterodimer-Promoting (K-coil) Domain (KVAACKE-KVAALKE-KVAALKE-KVAALKE (SEQ ID NO:85)); and a C-terminus. The amino acid sequence of such second polypeptide chain of DART-2 (SEQ ID NO:138) is provided below (the sequence of the hMAB-A VH (2) Domain (SEQ ID NO:17) is underlined; the sequence of the CD3 mAb-1 VL Domain (SEQ ID NO:115) is italicised):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGEV
QLVESGGGLV KPGGSLRLSC AASGFTFSSY WMHWVRQAPG
KGLEWVGEII PIFGHTNYNE KFKSRFTISL DNSKNTLYLQ
MGSLRAEDTA VYYCARGGYY YYGSRDYFDY WGQGTTVTVS
SASTKGKVAA CKEKVAALKE KVAALKEKVA ALKE

The third polypeptide chain of the exemplary ADAM9×CD3 bispecific three chain diabodies comprises, in the N-terminal to C-terminal direction: an N-terminus; a spacer peptide (DKTHTCPPCP (SEQ ID NO:93)); a hole-bearing IgG1 CH2-CH3 Domain (SEQ ID NO:110); and a C-terminus. Polynucleotides encoding this polypeptide chain may encode the C-terminal lysine residue of SEQ ID NO:110 (i.e., X of SEQ ID NO:110), however, as discussed above, this lysine residue may be post-translationally removed in some expression systems. Accordingly, the invention encompasses such a third polypeptide chain that contains such lysine residue (i.e., SEQ ID NO:110, wherein X is lysine), as well as a third polypeptide chain that lacks such lysine residue (i.e., SEQ ID NO:110, wherein X is absent). The amino acid sequence of such third polypeptide chain (SEQ ID NO:139) is provided below:

DKTHTCPPCP APEAAGGPSV FLFPPKPKDT LMISRTPEVT
CVVVDVSHED PEVKFNWYVD GVEVHNAKTK PREEQYNSTY
RVVSVLTVLH QDWLNGKEYK CKVSNKALPA PIEKTISKAK
GQPREPQVYT LPPSREEMTK NQVSLSCAVK GFYPSDIAVE
WESNGQPENN YKTTPPVLDS DGSFFLVSKL TVDKSRWQQG
NVFSCSVMHE ALHNRYTQKS LSLSPGX

wherein X is Lysine (K) or is absent.

It will be appreciated in view of the teachings provided herein that different domain orientations, VH Domains, VL Domains, linkers, and/or heterodimer promoting domains, could be utilized to generate alternative ADAM9×CD3 bispecific three chain diabodies. In particular, the VH Domain and VL Domain of different hMAB-A variants may be utilized.

### C. ADAM9×CD3×CD8 Trivalent Binding Molecules

Exemplary trivalent “ADAM9×CD3×CD8” binding molecules having one binding site specific for ADAM9 (comprising a parental and/or humanized anti-ADAM9-VL Domain and a corresponding anti-ADAM9-VH Domain, as described above), one binding site specific for CD3 (comprising, for example, the VL Domain of CD3 mAb-1 (SEQ ID NO:115) and the VH Domain of anti-CD3 antibody (e.g., CD3 mAb 1 (D65G) (SEQ ID NO:116)), and one binding site specific for CD8 (comprising, for example, the VH and VL Domains of TRX2 (SEQ ID NOs:123 and 124, respectively). Such trivalent binding molecules may have two polypeptide chains (see, e.g., FIG. 6E, and FIG. 6F), three polypeptide chains (see, e.g., FIG. 6C and FIG. 6D), four polypeptide chains (see, e.g., FIG. 6A and FIG. 6B), or five polypeptide chains (see, e.g., FIG. 5).

### XII. Methods of Production

The ADAM9-binding molecules of the present invention are most preferably produced through the recombinant expression of nucleic acid molecules that encode such polypeptides, as is well-known in the art.

Polypeptides of the invention may be conveniently prepared using solid phase peptide synthesis (Merrifield, B. (1986) “Solid Phase Synthesis,” Science 232(4748):341-347; Houghten, R. A. (1985) “General Method For The Rapid Solid-Phase Synthesis Of Large Numbers Of Peptides: Specificity Of Antigen-Antibody Interaction At The Level Of Individual Amino Acids,” Proc. Natl. Acad. Sci. (U.S.A.) 82(15):5131-5135; Ganesan, A. (2006) “Solid-Phase Synthesis In The Twenty-First Century,” Mini Rev. Med. Chem. 6(1):3-10).

In an alternative, antibodies may be made recombinantly and expressed using any method known in the art. Antibodies may be made recombinantly by first isolating the antibodies made from host animals, obtaining the gene sequence, and using the gene sequence to express the antibody recombinantly in host cells (e.g., CHO cells). Another method that may be employed is to express the antibody sequence in plants {e.g., tobacco) or transgenic milk. Suitable methods for expressing antibodies recombinantly in plants or milk have been disclosed (see, for example, Peeters et al. (2001) “Production Of Antibodies And Antibody Fragments In Plants,” Vaccine 19:2756; Lonberg, N. et al. (1995) “Human Antibodies From Transgenic Mice,” Int. Rev. Immunol 13:65-93; and Pollock et al. (1999) “Transgenic Milk As A Method For The Production Of Recombinant Antibodies,” J. Immunol Methods 231:147-157). Suitable methods for making derivatives of antibodies, e.g., humanized, single-chain, etc. are known in the art, and have been described above. In another alternative, antibodies may be made recombinantly by phage display technology (see, for example, U.S. Pat. Nos. 5,565,332; 5,580,717; 5,733,743; 6,265,150; and Winter, G. et al. (1994) “Making Antibodies By Phage Display Technology,” Annu. Rev. Immunol. 12.433-455).

Vectors containing polynucleotides of interest (e.g., polynucleotides encoding the polypeptide chains of the ADAM9-binding molecules of the present invention) can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

Any host cell capable of overexpressing heterologous DNAs can be used for the purpose of expressing a polypeptide or protein of interest. Non-limiting examples of suitable mammalian host cells include but are not limited to COS, HeLa, and CHO cells.

The invention includes polypeptides comprising an amino acid sequence of an ADAM9-binding molecule of this invention. The polypeptides of this invention can be made by procedures known in the art. The polypeptides can be produced by proteolytic or other degradation of the antibodies, by recombinant methods (i.e., single or fusion polypeptides) as described above or by chemical synthesis. Polypeptides of the antibodies, especially shorter polypeptides up to about 50 amino acids, are conveniently made by chemical synthesis. Methods of chemical synthesis are known in the art and are commercially available.

The invention includes variants of ADAM9-binding molecules, including functionally equivalent polypeptides that do not significantly affect the properties of such molecules as well as variants that have enhanced or decreased activity. Modification of polypeptides is routine practice in the art and need not be described in detail herein. Examples of modified polypeptides include polypeptides with conservative substitutions of amino acid residues, one or more deletions or additions of amino acids which do not significantly or deleteriously change the functional activity, or use of chemical analogs. Amino acid residues that can be conservatively substituted for one another include but are not limited to: glycine/alanine; serine/threonine; valine/isoleucine/leucine; asparagine/glutamine; aspartic acid/glutamic acid; lysine/arginine; and phenylalanine/tyrosine. These polypeptides also include glycosylated and non-glycosylated polypeptides, as well as polypeptides with other post-translational modifications, such as, for example, glycosylation with different sugars, acetylation, and phosphorylation. Preferably, the amino acid substitutions would be conservative, i.e., the substituted amino acid would possess similar chemical properties as that of the original amino acid. Such conservative substitutions are known in the art, and examples have been provided above. Amino acid modifications can range from changing or modifying one or more amino acids to complete redesign of a region, such as the Variable Domain. Changes in the Variable Domain can alter binding affinity and/or specificity. Other methods of modification include using coupling techniques known in the art, including, but not limited to, enzymatic means, oxidative substitution and chelation. Modifications can be used, for example, for attachment of labels for immunoassay, such as the attachment of radioactive moieties for radioimmunoassay. Modified polypeptides are made using established procedures in the art and can be screened using standard assays known in the art.

The invention encompasses fusion proteins comprising one or more of the anti-ADAM9-VL and/or VH of this invention. In one embodiment, a fusion polypeptide is provided that comprises a light chain, a heavy chain or both a light and heavy chain. In another embodiment, the fusion polypeptide contains a heterologous immunoglobulin constant region. In another embodiment, the fusion polypeptide contains a Light Chain Variable Domain and a Heavy Chain Variable Domain of an antibody produced from a publicly-deposited hybridoma. For purposes of this invention, an antibody fusion protein contains one or more polypeptide domains that specifically bind to ADAM9 and another amino acid sequence to which it is not attached in the native molecule, for example, a heterologous sequence or a homologous sequence from another region.

The present invention particularly encompasses ADAM9-binding molecules (e.g., antibodies, diabodies, trivalent binding molecules, etc.) conjugated to a diagnostic or therapeutic moiety. For diagnostic purposes, ADAM9-binding molecules of the invention may be coupled to a detectable substance. Such ADAM9-binding molecules are useful for monitoring and/or prognosing the development or progression of a disease as part of a clinical testing procedure, such as determining the efficacy of a particular therapy. Examples of detectable substances include various enzymes (e.g., horseradish peroxidase, beta-galactosidase, etc.), prosthetic groups (e.g., avidin/biotin), fluorescent materials (e.g., umbelliferone, fluorescein, or phycoerythrin), luminescent materials (e.g., luminol), bioluminescent materials (e.g., luciferase or aequorin), radioactive materials (e.g., carbon-14, manganese-54, strontium-85 or zinc-65), positron emitting metals, and nonradioactive paramagnetic metal ions. The detectable substance may be coupled or conjugated either directly to the ADAM9-binding molecule or indirectly, through an intermediate (e.g., a linker) using techniques known in the art.

For therapeutic purposes ADAM9-binding molecules of the invention may be conjugated to a therapeutic moiety such as a cytotoxin, (e.g., a cytostatic or cytocidal agent), a therapeutic agent or a radioactive metal ion, e.g., alpha-emitters. A cytotoxin or cytotoxic agent includes any agent that is detrimental to cells such as, for example, Pseudomonas exotoxin, Diptheria toxin, a botulinum toxin A through F, ricin abrin, saporin, and cytotoxic fragments of such agents. A therapeutic agent includes any agent having a therapeutic effect to prophylactically or therapeutically treat a disorder. Such therapeutic agents may be may be chemical therapeutic agents, protein or polypeptide therapeutic agents, and include therapeutic agents that possess a desired biological activity and/or modify a given biological response. Examples of therapeutic agents include alkylating agents, angiogenesis inhibitors, anti-mitotic agents, hormone therapy agents, and antibodies useful for the treatment of cell proliferative disorders. The therapeutic moiety may be coupled or conjugated either directly to the ADAM9-binding molecule or indirectly, through an intermediate (e.g., a linker) using techniques known in the art.

### XIII. Uses of the ADAM9-Binding Molecules of the Present Invention

The present invention encompasses compositions, including pharmaceutical compositions, comprising the ADAM9-binding molecules of the present invention (e.g., antibodies, bispecific antibodies, bispecific diabodies, trivalent binding molecules, etc.), polypeptides derived from such molecules, polynucleotides comprising sequences encoding such molecules or polypeptides, and other agents as described herein.

As provided herein, the ADAM9-binding molecules of the present invention, comprising the anti-ADAM9-VL and/or VH Domains provided herein, have the ability to bind ADAM9 present on the surface of a cell and induce antibody-dependent cell-mediated cytotoxicity (ADCC) and/or complement dependent cytotoxicity (CDC) and/or mediate redirected cell killing (e.g., redirected T-cell cytotoxicity).

Thus, ADAM9-binding molecules of the present invention, comprising the anti-ADAM9-VL and/or VH Domains provided herein, have the ability to treat any disease or condition associated with or characterized by the expression of ADAM9. As discussed above, ADAM9 is an onco-embryonic antigen expressed in numerous blood and solid malignancies that is associated with high-grade tumors exhibiting a less-differentiated morphology, and is correlated with poor clinical outcomes. Thus, without limitation, the ADAM9-binding molecules of the present invention may be employed in the diagnosis or treatment of cancer, particularly a cancer characterized by the expression of ADAM9.

In particular, ADAM9-binding molecules of the present invention may be used in the treatment of bladder cancer, breast cancer, cervical cancer, colorectal cancer, esophageal cancer, gastric cancer, head and neck cancer, liver cancer, lymphoid cancer, non-small-cell lung cancer, myeloid cancer ovarian cancer, pancreatic cancer, prostate cancer, renal cell carcinoma, thyroid cancer, testicular cancer, and uterine cancer.

In further embodiments, ADAM-9-binding molecules of the present invention may be useful in the treatment of non-small-cell lung cancer (squamous cell, adenocarcinoma, or large-cell undifferentiated carcinoma) and colorectal cancer (adenocarcinoma, gastrointestinal carcinoid tumors, gastrointestinal stromal tumors, primary colorectal lymphoma, leiomyosarcoma, melanoma, or squamous cell carcinoma).

The bispecific ADAM9-binding molecules of the present invention augment the cancer therapy provided by ADAM9 by promoting the redirected killing of tumor cells that express the second specificity of such molecules (e.g., CD2, CD3, CD8, CD16, the T Cell Receptor (TCR), NKG2D, etc.). Such ADAM9-binding molecules are particularly useful for the treatment of cancer.

In addition to their utility in therapy, the ADAM9-binding molecules of the present invention may be detectably labeled and used in the diagnosis of cancer or in the imaging of tumors and tumor cells.

### XIV. Pharmaceutical Compositions

The compositions of the invention include bulk drug compositions useful in the manufacture of pharmaceutical compositions (e.g., impure or non-sterile compositions) and pharmaceutical compositions (i.e., compositions that are suitable for administration to a subject or patient) that can be used in the preparation of unit dosage forms. Such compositions comprise a prophylactically or therapeutically effective amount of the ADAM9-binding molecules of the present invention, or a combination of such agents and a pharmaceutically acceptable carrier. Preferably, compositions of the invention comprise a prophylactically or therapeutically effective amount of the ADAM9-binding molecules of the present invention and a pharmaceutically acceptable carrier. The invention also encompasses such pharmaceutical compositions that additionally include a second therapeutic antibody (e.g., tumor-specific monoclonal antibody) that is specific for a particular cancer antigen, and a pharmaceutically acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The invention also provides a pharmaceutical pack or kit comprising one or more containers filled with an ADAM9-binding molecule of the present invention, alone or with such pharmaceutically acceptable carrier. Additionally, one or more other prophylactic or therapeutic agents useful for the treatment of a disease can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that can be used in the above methods. A kit can comprise any of the ADAM9-binding molecules of the present invention. The kit can further comprise one or more other prophylactic and/or therapeutic agents useful for the treatment of cancer, in one or more containers.

### XV. Methods of Administration

The compositions of the present invention may be provided for the treatment, prophylaxis, and amelioration of one or more symptoms associated with a disease, disorder or infection by administering to a subject an effective amount of an ADAM9-binding molecule (e.g. an antibody, bispecific antibody, diabody, trivalent binding molecule, fusion protein, etc.) or a conjugated ADAM9-binding molecule of the invention, or a pharmaceutical composition comprising an ADAM9-binding molecule or a conjugated ADAM9-binding molecule of the invention. In a preferred aspect, such compositions are substantially purified (i.e., substantially free from substances that limit its effect or produce undesired side effects). In a specific embodiment, the subject is an animal, preferably a mammal such as non-primate (e.g., bovine, equine, feline, canine, rodent, etc.) or a primate (e.g., monkey such as, a cynomolgus monkey, human, etc.). In a preferred embodiment, the subject is a human.

Various delivery systems are known and can be used to administer the compositions of the invention, e.g., encapsulation in liposomes, microparticles, microcapsules, recombinant cells capable of expressing the antibody or fusion protein, receptor-mediated endocytosis (See, e.g., Wu et al. (1987) “Receptor-Mediated In Vitro Gene Transformation By A Soluble DNA Carrier System,” J. Biol. Chem. 262:4429-4432), construction of a nucleic acid as part of a retroviral or other vector, etc.

Methods of administering an ADAM9-binding molecule of the invention include, but are not limited to, parenteral administration (e.g., intradermal, intramuscular, intraperitoneal, intravenous and subcutaneous), epidural, and mucosal (e.g., intranasal and oral routes). In a specific embodiment, the ADAM9-binding molecules of the present invention are administered intramuscularly, intravenously, or subcutaneously. The compositions may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968; 5,985,320; 5,985,309; 5,934,272; 5,874,064; 5,855,913; 5,290,540; and 4,880,078; and PCT Publication Nos. WO 92/19244; WO 97/32572; WO 97/44013; WO 98/31346; and WO 99/66903, each of which is incorporated herein by reference in its entirety.

The invention also provides that preparations of the ADAM9-binding molecules of the present invention are packaged in a hermetically sealed container such as an ampoule or sachette indicating the quantity of the molecule. In one embodiment, such molecules are supplied as a dry sterilized lyophilized powder or water free concentrate in a hermetically sealed container and can be reconstituted, e.g., with water or saline to the appropriate concentration for administration to a subject. Preferably, the ADAM9-binding molecules of the present invention are supplied as a dry sterile lyophilized powder in a hermetically sealed container.

The lyophilized preparations of the ADAM9-binding molecules of the present invention should be stored at between 2° C. and 8° C. in their original container and the molecules should be administered within 12 hours, preferably within 6 hours, within 5 hours, within 3 hours, or within 1 hour after being reconstituted. In an alternative embodiment, such molecules are supplied in liquid form in a hermetically sealed container indicating the quantity and concentration of the molecule, fusion protein, or conjugated molecule. Preferably, such ADAM9-binding molecules when provided in liquid form are supplied in a hermetically sealed container.

As used herein, an “effective amount” of a pharmaceutical composition is an amount sufficient to effect beneficial or desired results including, without limitation, clinical results such as decreasing symptoms resulting from the disease, attenuating a symptom of infection (e.g., viral load, fever, pain, sepsis, etc.) or a symptom of cancer (e.g., the proliferation, of cancer cells, tumor presence, tumor metastases, etc.), thereby increasing the quality of life of those suffering from the disease, decreasing the dose of other medications required to treat the disease, enhancing the effect of another medication such as via targeting and/or internalization, delaying the progression of the disease, and/or prolonging survival of individuals.

An effective amount can be administered in one or more administrations. For purposes of this invention, an effective amount of drug, compound, or pharmaceutical composition is an amount sufficient to: kill and/or reduce the proliferation of cancer cells, and/or to eliminate, reduce and/or delay the development of metastasis from a primary site of cancer. In some embodiments, an effective amount of a drug, compound, or pharmaceutical composition may or may not be achieved in conjunction with another drug, compound, or pharmaceutical composition. Thus, an “effective amount” may be considered in the context of administering one or more chemotherapeutic agents, and a single agent may be considered to be given in an effective amount if, in conjunction with one or more other agents, a desirable result may be or is achieved.

For the ADAM9-binding molecules encompassed by the invention, the dosage administered to a patient is preferably determined based upon the body weight (kg) of the recipient subject.

The dosage and frequency of administration of an ADAM9-binding molecule of the present invention may be reduced or altered by enhancing uptake and tissue penetration of the molecule by modifications such as, for example, lipidation.

The dosage of an ADAM9-binding molecule of the invention administered to a patient may be calculated for use as a single agent therapy. Alternatively, the molecule may be used in combination with other therapeutic compositions and the dosage administered to a patient are lower than when said molecules are used as a single agent therapy.

The pharmaceutical compositions of the invention may be administered locally to the area in need of treatment; this may be achieved by, for example, and not by way of limitation, local infusion, by injection, or by means of an implant, said implant being of a porous, non-porous, or gelatinous material, including membranes, such as SILASTIC® membranes, or fibers. Preferably, when administering a molecule of the invention, care must be taken to use materials to which the molecule does not absorb.

The compositions of the invention can be delivered in a vesicle, in particular a liposome (See Langer (1990) “New Methods Of Drug Delivery,” Science 249:1527-1533); Treat et al., in LIPOSOMES IN THE THERAPY OF INFECTIOUS DISEASE AND CANCER, Lopez-Berestein and Fidler (eds.), Liss, New York, pp. 353-365 (1989); Lopez-Berestein, ibid., pp. 3 17-327).

Where the composition of the invention is a nucleic acid encoding an ADAM9-binding molecule of the present invention, the nucleic acid can be administered in vivo to promote expression of its encoded ADAM9-binding molecule by constructing it as part of an appropriate nucleic acid expression vector and administering it so that it becomes intracellular, e.g., by use of a retroviral vector (See U.S. Pat. No. 4,980,286), or by direct injection, or by use of microparticle bombardment (e.g., a gene gun; Biolistic, Dupont), or coating with lipids or cell surface receptors or transfecting agents, or by administering it in linkage to a homeobox-like peptide which is known to enter the nucleus (See e.g., Joliot et al. (1991) “Antennapedia Homeobox Peptide Regulates Neural Morphogenesis,” Proc. Natl. Acad. Sci. (U.S.A.) 88:1864-1868), etc. Alternatively, a nucleic acid can be introduced intracellularly and incorporated within host cell DNA for expression by homologous recombination.

### EXAMPLES

Having now generally described the invention, the same will be more readily understood through reference to the following Examples. The following examples illustrate various methods for compositions in the diagnostic or treatment methods of the invention. The examples are intended to illustrate, but in no way limit, the scope of the invention.

### Example 1

### Tumor Cell Specificity of the Anti-ADAM9 Antibody MAB-A

A murine anti-ADAM9 antibody (designated herein as MAB-A) was identified that: (1) blocks the target protein processing activity of ADAM9; (2) is internalized; and (3) has anti-tumor activity (see, e.g., U.S. Pat. No. 8,361,475). The tumor cell specificity of MAB-A was investigated by IHC. Tumor tissue was contacted with MAB-A (0.4 μg/mL) or an isotype control (0.4 μg/mL) and the extent of staining was visualized. MAB-A was found to strongly label a variety of large cell carcinoma, squamous cell carcinoma, and adenocarcinoma non-small cell lung cancer cell types (FIG. 7A, Panels 1-8), breast cancer cells, prostate cancer cells, gastric cancer cells (FIG. 7B, Panels 1-6), as well as colon cancer samples (FIG. 7C, Panels 1-8). Normal tissue was contacted with MAB-A (1.25 μg/mL) and the extent of staining was visualized. As summarized in Table 1 above, MAB-A exhibited little or no staining of a wide variety of normal tissues. It will be noted that the concentration of MAB-A used in these studies was nearly 3-times that used for staining of tumor cells. The results of these IHC studies indicate that MAB-A exhibits strong preferential binding to tumor cells over normal cells.

### Example 2

### Species Cross Reactivity

The binding of MAB-A to human ADAM9 (huADAM9) and cynomolgus monkey ADAM9 (cynoADAM9) was examined. Briefly, 293-FT and CHO-K cells transiently expressing huADAM9, cynoADAM9, an unrelated antigen, or the untransfected parental cells were incubated with MAB-A followed by goat anti-murine-PE secondary antibody and analyzed by FACS. As shown in FIGS. 8A-8B, MAB-A exhibited strong binding to huADAM9 transiently expressed on both cells types. MAB-A exhibited poor binding to cynoADAM9. MAB-A did not bind to the parental cells or cells expressing an irrelevant antigen. Similar poor binding to cynoADAM were seen in ELISA assays.

### Example 2

### Humanization and Initial Optimization

Humanization of MAB-A yielded a humanized VH Domain, designated herein as “hMAB-A VH(1)” and a humanized VL Domain designated herein as “hMAB-A VL(1).” The humanized Variable Domains were then optimized to enhance binding activity and/or to remove potentially labile amino acid residues as described in more detail below. This first round of optimization yielded three additional humanized VH Domains, designated herein as “hMAB-A VH(2),” “hMAB-A VH(3),” and “hMAB-A VH(4),” and three additional humanized VL Domains designated herein as “hMAB-A VL(2),” “hMAB-A VL(3),” and “hMAB-A VL(4).” In addition, a chimeric version of MAB-A (“chMAB-A”) having the murine VH and VL Domains and human constant regions was generated. The amino acid sequences of the murine and the humanized/optimized VH and VL Domains are provided above, an alignment is provided in FIGS. 9A and 9B. The consensus sequence of these humanized/optimized VH and VL Domains is provided above. Where multiple humanized Variable Domains were generated the humanized heavy and light chain Variable Domains of a particular anti-ADAM9 antibody (e.g., MAB-A) may be used in any combination and particular combinations of humanized chains are referred to by reference to the specific VH/VL Domains, for example a molecule (e.g., an antibody or diabody) comprising hMAB-A VH(1) and hMAB-A VL(2) is specifically referred to as “hMAB-A (1.2).”

hMAB-A VH(1) was generated having framework regions derived from human germlines VH3-21 and VH3-64, and hMAB-A VL(1) was generated having framework regions derived from human germlines B3 and L6. The murine CDRs were retained in these humanized variable domains.

A potential deamidation site was identified in the CDR_{H}2 (shown in single underlining in FIG. 9A) and a potential aspartic acid isomerization site was identified in CDR_{L}1 (shown in single underlining in FIG. 9B). Amino acid substitutions at these positions were examined to identify substitutions to remove these sites while maintaining binding affinity. A substitution of phenylalanine at position 54 (N54F) of CDR_{H}2 (present in hMAB-A VH(2)) and at serine at position 28 (D28S) of CDR_{L}1 (present in hMAB-A VL(2)) were selected, wherein the numbering is accordingly to Kabat. The identified substitutions may be used separately or in combination. Surprisingly, antibodies comprising the N54F substitution were found to exhibit about a 2-fold increase in affinity for human ADAM9, and to exhibit slightly improved binding to cynomolgus ADAM9.

Additional, optimized variants were generated to minimize the number of lysine residues present in the CDRs. Two lysine residues are present in CDR_{H}2 (indicated with a double underline in FIG. 9A), and one lysine is present in CDR_{L}1 (indicated with a double underline in FIG. 9B). Amino acid substitutions at these positions were examined to identify substitutions that maintained binding affinity. Substitutions of arginine at position 62 (K62R), of glutamine at position 64 (K64Q), and serine at position 65 (S65G) were selected for CDR_{H}2 (present in hMAB-A VH(3)), wherein the numbering is accordingly to Kabat. A substitution of an arginine at position 24 (K24R) was selected for CDR_{L}1 (present in hMAB-A VL(3)). The identified substitutions may be used separately or in combination.

Other potentially labile resides present in the CDRs were identified (indicated with a dotted underline in FIGS. 9A-9B), one methionine residue within CDR_{H}1 at position 34 (M34), one methionine residue within CDR_{L}1 at position 33 (M33), and histidine, glutamic acid, and aspartic acid residues at positions 92 (H93), 93 (E93), and 94 (D94), within CDR_{L}3, wherein the numbering is accordingly to Kabat. Amino acid substitutions at these positions were examined to identify substitutions that maintained binding affinity. Substitution of isoleucine at position 34 (M34I) was selected for CDR_{H}1 and substitutions of leucine, tyrosine, serine and threonine were selected for positions 33 (M33L), 92 (H93Y), 93 (E93S), and 94 (D94T) of CDR_{L}3, wherein the numbering is according to Kabat. Each of these positions could readily be substituted in combination with all of the substitutions detailed above to yield hMAB-A VH(4) and hMAB-A VL(4), which when paired together generate an antibody that retained a small improvement in affinity as compared to the parental murine antibody, and that has a greatly reduced potential for deamidation or oxidation and no lysine residues in the CDRs.

The relative binding affinity of the humanized/optimized antibodies hMAB-A (1.1), hMAB-A (2.2), hMAB-A (2.3), hMAB-A (3.3), hMAB-A (4.4) and the chimeric chMAB-A (having murine VH/VL Domains) to huADAM was investigated using BIACORE® analysis, in which His-tagged soluble human ADAM9 (“shADAM9-His,” containing an extracellular portion of human ADAM9 fused to a histidine-containing peptide) was passed over a surface coated with immobilized antibody. Briefly, each antibody was captured onto an Fab₂ goat-anti-human Fc-coated surface and then incubated in the presence of different concentrations (6.25-100 nM) of the shADAM9-His peptide. The kinetics of binding were determined via BIACORE® analysis binding (normalized 1:1 Langmuir binding model). The calculated kₐ, k_{d} and K_{D} from these studies are presented in Table 7. Binding to cynoADAM9 was examined by FACS as described above and by ELISA.

TABLE 7
Table 7. Binding to cynoADAM9 was examined
by FACS as described above and by ELISA.

| | | huADAM9 | | |
|---|---|---|---|---|
| Antibody | pI | kₐ (×10⁶) | k_{d} (×10⁻³) | KD (nM) |
| chMAB-A | 6.61 | 1.3 | 4.7 | 3.6 |
| hMAB-A (1.1) | 6.44 | 1.5 | 5.2 | 3.5 |
| hMAB-A (2.2) | 6.58 | 1.1 | 1.5 | 1.4 |
| hMAB-A (2.3) | 6.58 | 1.3 | 1.7 | 1.3 |
| hMAB-A (3.3) | 6.44 | 1.1 | 1.5 | 1.4 |
| hMAB-A (4.4) | 6.73 | 1.0 | 2.0 | 2.0 |

The results of these studies demonstrate that the humanized/optimized antibodies have the same or higher binding affinity to human ADAM9 than the parental murine antibody. In particular, it was observed that the introduction of the N54F mutation in the humanized antibodies resulted in improved binding to huADAM9 (i.e., hMAB-A (2.2), hMAB-A (2.3) and hMAB-A (3.3)). This mutation also provided a slight improvement in binding to cynoADAM9 as determined by FACS and ELISA, however these antibodies continued to exhibit poor binding to cynoADAM9. These studies also identified additional substitutions that could be introduced to remove lysine residues from the CDRs without reducing affinity. Additional substitutions were identified to remove other potentially labile residues with a minimal impact on affinity.

### Example 4

### Optimization of Binding to Non-Human Primate ADAM9

Random mutagenesis was used to introduce substitutions within the Heavy Chain CDR_{H}2 (Kabat positions 53-58) and CDR_{H}3 (Kabat positions 95-100 and 100a-100f) domains of hMAB-A (2.2). The mutants were screened to identify clones having enhanced binding to non-human primate ADAM9 (e.g., cynoADAM9) and that retained high affinity binding to huADAM9. 48 clones were selected from two independent screens of mutations within CDR_{H}3 (Kabat positions 100a-100f). Table 8 provides an alignment of the amino acid sequence of CDR_{H}3 Kabat residues 100a-f from hMAB-A (2.2) clones selected for enhanced binding to cynoADAM9 from two independent screens. Additional clone alignments are provided in Table 9. As indicated in such Tables, similar clones emerged in each experiment, which fell into discrete substitution patterns.

TABLE 8
Substitutions within Sub-Domain of the Heavy Chain CDRH3 of MAB-A
(Kabat Positions 100a-100f)

| Screen 1 | | | Screen 2 | | |
|---|---|---|---|---|---|
| | | CDR_{H}3 | | | CDR_{H}3 |
| | | Sub- | | | Sub- |
| | | Domain | | | Domain |
| Clone ID | SEQ ID NO | Sequence | Clone ID | SEQ ID NO | Sequence |
| MAB-A | 140 | GSRDYF | MAB-A | 140 | GSRDYF |
| 1 | 141 | DGEGVM | 1 | 171 | DGKAVL |
| 2 | 141 | DGEGVM | 2 | 172 | FNKAVL |
| 3 | 142 | FHSGLL | 3 | 143 | FNSATL |
| 4 | 143 | FNSATL | 4 | 173 | FNSGTW |
| 5 | 144 | FNSGTL | 5 | 174 | FNTGVF |
| 6 | 145 | FNSSTL | 6 | 175 | GKSRFH |
| 7 | 146 | GKSKWL | 7 | 150 | IGKGVF |
| 8 | 147 | GMGGTL | 8 | 151 | IGKGVL |
| 9 | 148 | HAKGGM | 9 | 176 | IGKNVY |
| 10 | 149 | IGEAVL | 10 | 177 | MGKGVM |
| 11 | 150 | IGKGVF | 11 | 178 | NGESVF |
| 12 | 150 | IGKGVF | 12 | 179 | PDFGWM |
| 13 | 151 | IGKGVL | 13 | 180 | PGSGVM |
| 14 | 152 | KHDSVL | 14 | 181 | PKDAWL |
| 15 | 153 | LNTAVM | 15 | 158 | PKFGWK |
| 16 | 154 | NGEGTL | 16 | 158 | PKFGWK |
| 17 | 155 | NGKNTL | 17 | 182 | PKFGWL |
| 18 | 156 | NSAGIL | 18 | 183 | PKIGWH |
| 19 | 157 | PKEGWM | 19 | 183 | PKIGWH |
| 20 | 158 | PKFGWK | 20 | 183 | PKIGWH |
| 21 | 159 | PKMGWV | 21 | 184 | PKMGWA |
| 22 | 160 | PRLGHL | 22 | 185 | PKMGWM |
| 23 | 161 | PSFGWA | 23 | 185 | PKMGWM |
| 24 | 162 | QAKGTM | 24 | 185 | PKMGWM |
| 25 | 163 | RGMGVM | 25 | 185 | PKMGWM |
| 26 | 164 | RKEGWM | 26 | 186 | PQMGWL |
| 27 | 165 | TGKGVL | 27 | 187 | PRFGWL |
| 28 | 166 | TGMGTL | 28 | 187 | PRFGWL |
| 29 | 167 | TGNGVM | 29 | 187 | PRFGWL |
| 30 | 167 | TGNGVM | 30 | 188 | PRMGFL |
| 31 | 168 | WNAGTF | 31 | 189 | PRMGFM |
| 32 | 169 | YHHTPL | 32 | 190 | PSFGWM |
| 33 | 169 | YHHTPL | 33 | 191 | RREGWM |
| 34 | 170 | YQSATL | 34 | 192 | SGEGVL |
| | | | 35 | 193 | SGNGVM |
| | | | 36 | 194 | VGKAVL |

TABLE 9
Substitutions within Sub-Domain of the Heavy
Chain CDRH3 of MAB-A
(Kabat Positions 100a-100f)

| | | CDR_{H}3 |
|---|---|---|
| | | Sub-Domain |
| Clone ID | SEQ ID NO | Sequence |
| MAB-A VH (2A) | 144 | FNSGTL |
| MAB-A VH (2B) | 151 | IGKGVL |
| MAB-A VH (2C) | 187 | PRFGWL |
| MAB-A VH (2D) | 165 | TGKGVL |
| MAB-A VH (2E) | 195 | DSNAVL |
| MAB-A VH (2F) | 196 | FHSGTL |
| MAB-A VH (2G) | 172 | FNKAVL |
| MAB-A VH (2H) | 197 | GGSGVL |
| MAB-A VH (2I) | 198 | PRQGFL |
| MAB-A VH (2J) | 199 | YNSGTL |

For all the clones examined, Gly and Ala are the preferred amino acid residues at positon 4 (P4) and Leu, Met, and Phe are the preferred amino acid residues at position 6 (P6). The preferred amino acid residues at other positions (e.g., position 2 (P2), position 3 (P3) and position 5 (P5)) depend on the amino acid residue found at P1. For clones having a Pro residue at position 1 (P1), Lys and Arg were preferred at P2, Phe and Met at P3, Gly at P4, and Trp or Phe at P5. For clones having a Phe, Tyr or Trp at P1, Asn and His were preferred at P2, Ser and His at P3, and Leu at P6. For clones having Ile, Leu or Val at P1, Gly was preferred at P2, Lys at P3, Val at P5 and hydrophobic at P6. In addition, as can be seen in Table 8, for clones having a Thr residue at P1, Gly was preferred at P2, Lys, Met, and Asn were preferred at P3, Gly was preferred at P4, Val or Thr were preferred at P5 and Leu and Met at P6. Additional clones having an Asp, Gly, Arg, His, or Ser residue at P1 were also identified at lower frequencies (see Table 8 and Table 9).

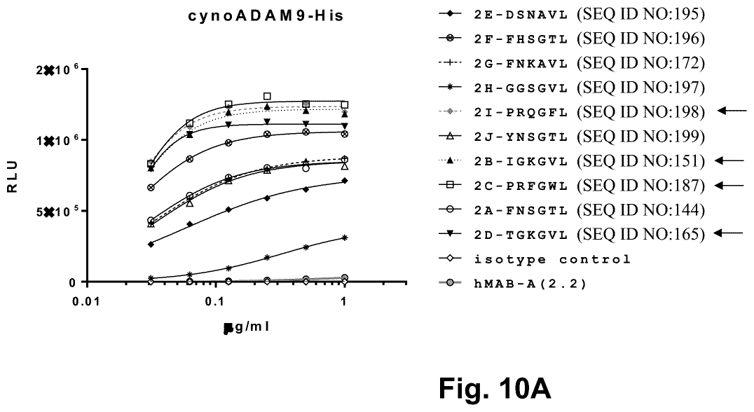

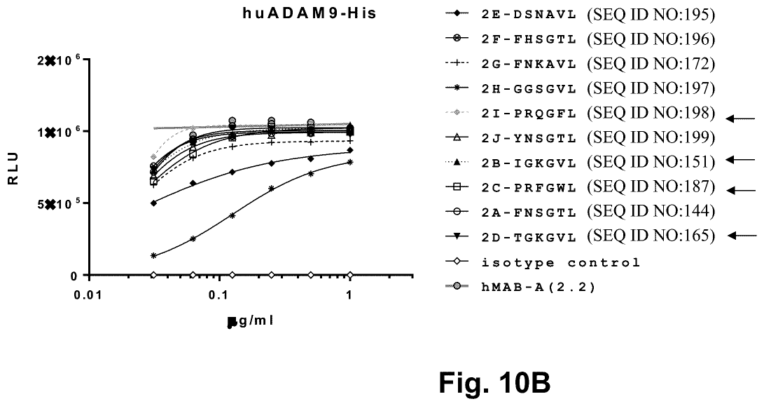

The VH Domain of the ten clones shown in Table 9 were used to generate further optimized variants of hMAB-A (2.2) designated hMAB-A (2A.2). The binding of the selected clones was examined by ELISA assay. Briefly, antibodies that bind to histidine-containing peptides, and that had been coated onto microtiter plates, were used to capture His peptide-tagged soluble cynoADAM9 (“cynoADAM9-His”) (1 μg/mL) or His peptide-tagged soluble huADAM9 (1 μg/mL), and the binding of serial dilutions of the parental hMAB-A (2.2) and the ten CDR_{H}3 hMAB-A (2A.2) variants was examined. The binding curves for cynoADAM9 and huADAM9 are presented in FIG. 10A and FIG. 10B, respectively. hMAB-A (2A.2) variants comprising each of the selected VH Domains exhibited improved binding to cynoADAM9 with MAB-A VH(2B), MAB-A VH(2C), MAB-A VH(2D), and MAB-A VH(2I), showing the greatest enhancement in cynoADAM9 binding while maintaining similar binding to huADAM9 as the parental hMAB-A (2.2) antibody.

The relative binding affinity of the humanized/further optimized antibodies MAB-A VH(2B.2), MAB-A VH(2C.2), MAB-A VH(2D.2), and MAB-A VH(21.2), and the parental hMAB-A (2.2), to huADAM9-His and cynoADAM9-His was investigated using BIACORE® analysis essentially as described above. The calculated kₐ, k_{d} and K_{D} from these studies are presented in Table 10.

TABLE 10

| | huADAM9 | | | cynoADAM9 | | |
|---|---|---|---|---|---|---|
| | kₐ (×10⁵) | k_{d} (×10⁻⁴) | KD | kₐ (×10⁵) | k_{d} (×10⁻⁴) | KD |
| Antibody | (M⁻¹s⁻¹) | (s⁻¹) | (nM) | (M⁻¹s⁻¹) | (s⁻¹) | (nM) |
| hMAB-A (2.2) | 9.0 | 5.5 | 0.6 | 2.0 | 220 | 110 |
| hMAB-A (2B.2) | 6.1 | 3.9 | 0.6 | 3.4 | 0.66 | 0.2 |
| hMAB-A (2C.2) | 5.9 | 8.1 | 1.4 | 3.5 | <0.1 | <0.3 |
| hMAB-A (2D.2) | 6.9 | 5.8 | 0.8 | 4.2 | 3.0 | 0.7 |
| hMAB-A (2I.2) | 6.6 | 2.3 | 0.4 | 4.0 | 0.85 | 0.2 |

The binding studies demonstrate that the four top clones exhibited between 150-550-fold enhancement in binding affinity to cynoADAM9 while maintaining the same high affinity binding to huADAM9 as the parental antibody. hMAB-A (2C.2) and hMAB-A (2I.2) was selected for further studies.

### Example 5

Immunohistochemistry Study of Antibody hMAB-A (2I.2)

The cell specificity of hMAB-A (2I.2) was investigated by IHC. Positive and negative control cells, and normal human and cynomolgus monkey tissues were contacted with hMAB-A (2I.2) (2.5 μg/mL) or an isotype control (2.5 μg/mL) and the extent of staining was visualized. The results of the study are summarized in Table 11.

TABLE 11

| | | IgG1 |
|---|---|---|
| | hMAB-A | Negative |
| | (2I.2) | Control |
| Cell/Tissue | (2.5 μg/ml) | (2.5 μg/ml) |
| Cho-K parental cells | — | — |
| Cho-K/huADAM9 | 2-4+ (gr c > m) | — |
| medium expression P:1 | rare to occasional | |
| | and 1+ (gr c > m) | |
| | occasional | |
| Cho-K/huADAM9 high | 2-4+ (gr c > m) | — |
| expression | frequent | |
| Cho-K/cynoADAM9 | 2-4+ (gr c > m) | — |
| clone 2 | frequent | |
| Cho-K/cynoADAM9 | 2-4+ (gr c > m) | — |
| clone #16 | frequent | |
| A498 cells | 2-4+ (gr c > m) | — |
| | rare to occasional | |
| | and 1+ (gr c > m) | |
| | occasional | |
| | to frequent | |
| Colon | — | numerous |
| MG06-CHTN-96 B | | 2-4+ (gr c) |
| | | cells consistent |
| | | with macrophages |
| Lung MG06-CHtN- | — | occasional |
| 162B1 A | | 2-4+ (gr c) |
| | | cells consistent |
| | | with macrophages |
| Liver ILS11103 B | — | hepatocytes |
| | | 1+ (gr c) |
| | | rare to occasional |
| Pancreas ILS10266 | — | — |
| Heart Life Legacy | — | cardiac muscle |
| 0910035D | | cells with numerous |
| | | 1-3+ |
| | | small foci of (gr c) |
| | | consistent with |
| | | lipofuscin pigment |
| Kidney ILS10241 B | — | tubule epi |
| | | 1+ (gr c) |
| | | rare |
| Bladder ILSD8011 J | — | occasional |
| | | 2-4+ (gr c) |
| | | cells consistent |
| | | with macrophages |
| Cyno Colon #1 | — | mucosal epi |
| | | (luminal m) |
| | | 2-4+ |
| | | rare to occasional |
| | | and 1+ |
| | | rare to occasional; |
| | | numerous |
| | | 2-3+ (gr c) |
| | | cells consistent |
| | | with macrophages |
| | | predominantly |
| | | within LP |
| Cyno Lung #1 | — | very rare |
| | | 2-4+ (gr c) |
| | | cells consistent |
| | | with macrophages |
| Cyno Liver #1 | — | — |
| Cyno Pancreas #1 | — | — |
| Cyno Heart #1 | — | — |
| Cyno Kidney #070368M | — | tubule epi |
| | | 2+ (gr c) |
| | | rare |
| | | and 1+ (gr c) |
| | | rare to occasional |
| Cyno Bladder #1 | transitional | rare |
| | cell epi ± | 1-4+ (gr c) |
| | (gr c) | cells consistent |
| | rare | with macrophages |
| Lung CA ILS10108 | H score 150 | tu− |
| Lung CA ILS7223 | H score 180 | tu− |
| Lung CA ILS2156 A | H score 80 | tu− |
| Lung CA ILS7295 A | H score 60 | tu− |

IHC studies were also conducted to assess binding of humanized/optimized hMAB-A (21.2) at a concentration of 12.5 μg/mL (5× optimal staining concentration). Positive and negative control cells, normal human tissues, and cynomolgus monkey tissues were employed in this study. The results of the study are summarized in Table 12.

TABLE 12

| | | IgG1 |
|---|---|---|
| | hMAB-A | Negative |
| | (2I.2) | Control |
| Cell/Tissue | (12.5 μg/ml) | (12.5 μg/ml) |
| Cho-K parental cells | — | — |
| Cho-K/huADAM9 | 2-4+ (gr c > m) | — |
| medium expression P:1 | occasional | |
| | to frequent | |
| Cho-K/huADAM9 high | 3-4+ (gr c > m) | — |
| expression | occasional | |
| | to frequent | |
| Cho-K/cynoADAM9 | 3-4+ (gr c > m) | — |
| clone 2 | frequent | |
| Cho-K/cynoADAM9 | 3-4+ (gr c > m) | — |
| clone #16 | frequent | |
| A498 cells | 2-4+ (gr c > m) | — |
| | occasional | |
| | to frequent | |
| Colon | epi ±− 1+ | numerous |
| MG06-CHTN-96 B | rare to occasional | 2-4+ (gr c) |
| | | cells consistent |
| | | with macrophages |
| | | predominantly |
| | | within LP in |
| | | test article and |
| | | negative control |
| Lung MG06-CHtN- | alveolar cells | occasional scattered |
| 162B1 A | (favor pneumocytes) | 2-4+ (gr c) |
| | 2-3+ (gr c > m) | cells consistent |
| | rare, | with macrophages |
| | 1+ (gr c > m) | in test article |
| | rare to occasional; | and negative control |
| | EC 2-4+ (c, m) | |
| | rare, | |
| | 1+ (c, m) | |
| | rare | |
| Liver ILS11103 B | — | occasional scattered |
| | | 2-4+ (gr c) |
| | | cells consistent |
| | | with macrophages |
| | | in test article |
| | | and negative control |
| Pancreas ILS10266 | ductal epi | cells (favor acinar cells) |
| | 1+ (gr c > m) | 1+ (gr c) |
| | very rare | very rare; |
| | | occasional scattered |
| | | 2-4+ (gr c) |
| | | cells consistent |
| | | with macrophages |
| | | in test article |
| | | and negative |
| Heart Life Legacy | — | numerous small foci |
| 0910035D | | 1-3+ |
| | | granular staining with |
| | | cardiac muscle cells |
| | | consistent with |
| | | lipofuscin pigment |
| | | consistent with |
| | | artifact in test article |
| | | and negative control |
| Kidney ILS10241 B | tubule epi | tubule epi ± |
| | 1+ (gr c) | (gr c) |
| | rare to occasional | rare |
| Bladder ILSD8011 J | transitional | rare |
| | cell epi | 2-4+ (gr c) |
| | 1+ (gr c) | cells consistent |
| | rare | with macrophages |
| | | in test article |
| | | and negative control |
| Cyno Colon #1 | — | mucosal epi (luminal m) |
| | | 2-4+ |
| | | occasional |
| | | and 1+ |
| | | rare to occasional |
| Cyno Lung #1 | bronchial epi | — |
| | 1+ (gr c > m) | |
| | rare to occasional | |
| | and ± (gr c > m) | |
| | occasional | |
| | to frequent | |
| Cyno Liver #1 | — | — |
| Cvno Pancreas #1 | — | — |
| Cyno Heart #1 | — | — |
| Cvno Kidney #070368M | — | tubule epi |
| | | 1+ (gr c) |
| | | rare |
| | | and ± (gr c) |
| | | rare |
| Cyno Bladder #1 | transitional cell epi | — |
| | 2+ (gr c > m) | |
| | rare | |
| | and 1+ (gr c > m) | |
| | rare to occasional | |
| Lung CA ILS10108 | H score 180 | tu- |
| Lung CA ILS7223 | H score 180 | tu- |
| Limg CA ILS2156 A | H score 115 | tu- |
| Lung CA ILS7295 A | H score 115 | tu- |

A comparative IHC study was conducted in order to assess differences in binding by hMAB-A (2.2), hMAB-A (2.3), hMAB-A (2C.2), and hMAB-A (21.2) at 2.5 g/mL or 5 μg/mL. Positive and negative control cells, normal human tissues, and cynomolgus monkey tissues were employed in this study. The results of the study are summarized in Table 13.

TABLE 13

| | hMAB-A | hMAB-A | hMAB-A | hMAB-A | Isotype |
|---|---|---|---|---|---|
| | (2.3) | (2.2) | (2C.2) | (2I.2) | control |
| Tissue | 5 μg/mL | 2.5 μg/mL | 2.5 μg/mL | 2.5 μg/mL | 5 μg/mL |
| Cho-K parental | — | — | — | — | — |
| P:3 | | | | | |
| Cho-K/hu | 1+ (c) | 2-4+ (gr c > m) | 2-4+ (gr c > m) | 2-4+ (gr c > m) | — |
| ADAM9.2 | occasional | rare | rare to occasional | rare to occasional | |
| medium | | and 1+ (gr c > m) | and 1+ (gr c > m) | and 1+ (gr c > m) | |
| expression P:1 | | rare to occasional | rare to occasional | occasional | |
| Cho-K/hu | 3+ (m, c) | 2-4+ (gr c > m) | 2-4+ (gr c > m) | 2-4+ (gr c > m) | — |
| ADAM9.18 high | frequent | occasional to | occasional to | frequent | |
| expression P:1 | | frequent | frequent | | |
| | | and 1+ (gr c > m) | and 1+ (grc > m) | | |
| | | occasional | occasional | | |
| Cho-K Cyno #2 | 1+ (c) | — | 3-4+ (gr c > m) | 2-4+ (gr c > m) | — |
| | occasional | | frequent | frequent | |
| Cho-K Cyno | 2+ (c, m) | 2-4+ (gr c > m) | 3-4+ (gr c > m) | 2-4+ (gr c > m) | — |
| #16 | occasional to | rare | frequent | frequent | |
| | frequent | and 1+ (gr c > m) | | | |
| | | rare to occasional | | | |
| A498 072210 | 3-4+ (c, m) | 2-4+ (gr c > m) | 2-4+ (gr c > m) | 2-4+ (gr c > m) | — |
| | frequent | rare | rare | rare to occasional | |
| | | and 1+ (gr c > m) | and 1+ (gr c > m) | and 1+ (gr c > m) | |
| | | occasional to | occasional | occasional to | |
| | | frequent | | frequent | |
| Lung CA | IHC score 3 | H Score 55 | H Score 17 | H score 150 | — |
| ILS10108 | | | | | |
| Lung CA | IHC score 3 | H Score 205 | H Score 160 | H score 180 | — |
| ILS7223 | | | | | |
| Lung CA | IHC score 1 | H Score 5 | H Score 0 | H score 80 | — |
| ILS2156 A | | | | | |
| Lung CA | IHC score 1 | H Score 1 | H Score 0 | H score 60 | — |
| ILS7295 A | | | | | |

A further comparative IHC study was conducted in order to assess differences in binding by hMAB-A (2.2), hMAB-A (2.3), hMAB-A (2C.2), and hMAB-A (21.2) and murine MAB-A at 2.5 μg/mL 5 μg/mL or 12.5 μg/mL. Positive and negative control cells, normal human tissues, and cynomolgus monkey tissues were employed in this study. The results of the study are summarized in Table 14.

TABLE 14

| | hMAB-A | hMAB-A | hMAB-A | hMAB-A | |
|---|---|---|---|---|---|
| | (2.3) | (2.2) | (2C.2) | (2I.2) | MAB-A |
| Tissue | 5 μg/mL | 2.5 μg/mL | 2.5 μg/mL | 12.5 μg/ml | 5 μg/mL |
| Colon | epi | — | — | epi ±− 1+ | Epithelium |
| MG06- | 1+ (c, m) | | | rare to occasional | 1-3+ [m, c] |
| CHTN-96 B | rare; sm negative | | | | (occas to freq); |
| | | | | | Others (Neg) |
| Lung | pneiunocytes/ | — | — | alveolar cells | Monoctves |
| MG06-CHtN- | macrophages | | | (favor pneumocytes) | 1+ [c] |
| 162B1 A | 2+ (c, m) | | | 2-3+ (gr c > m) | (rare to occas); |
| | occasional | | | rare, | Others (Neg) |
| | | | | 1+ (gr c > m) | |
| | | | | rare to occasional; | |
| | | | | EC 2-4+ (c, m) | |
| | | | | rare, | |
| | | | | 1+ (c, m) | |
| | | | | rare | |
| Liver | hepatocytes | hepatocytes | hepatocytes | — | Kupffer cells |
| ILS11103 B | 1+ (c) | 1+ (gr c) | 2+ (gr c) | | 3+ [c] |
| | rare to occasional | frequent | rare | | (occas); |
| | | | and 1+ (gr c) | | Others (Neg) |
| | | | frequent | | |
| Pancreas | epi 1+ (c) | — | — | ductal epi | Ductal epithelium |
| ILS10266 | rare: | | | 1+ (gr c > m) | 1-2+ [c, m] |
| | Islet Cells | | | very rare | (rare to occas); |
| | 1+ (c) | | | | Fibril |
| | very rare | | | | 2+ |
| | | | | | (rare); Others |
| | | | | | (Neg) |
| Heart Life | ± | — | — | — | Neg |
| Legacy | | | | | |
| 0910035D | | | | | |
| Kidney | epi 2-3+ (c, m) | tubule epi | tubule epi | tubule epi | Epithelium |
| ILS10241 B | frequent | 2+ (gr c) | 2+ (gr c) | 1+ (gr c) | 1+ [c] |
| | | rare to occasional | rare to occasional | rare to occasional | (rare); Others |
| | | and 1+ (gr c) | and 1+ (gr c) | | (Neg) |
| | | occasional | occasional | | |
| | | to frequent | to frequent | | |
| Bladder | transitional epi | — | — | transitional cell epi | Transitional |
| ILSD8011 J | 1+ (c) | | | 1+ (gr c) | epithelium |
| | rare to occasional | | | rare | 2+ [c, m] |
| | | | | | (occas to freq); |
| | | | | | Stromal cells |
| | | | | | 3+ [c] |
| | | | | | (rare); |
| | | | | | Others (Neg) |
| Cyno Colon | epi | — | — | — | |
| #1 | 1+ (c, m) | | | | |
| | rare | | | | |
| Cyno Lung #1 | Macrophage and | — | bronchial epi | bronchial epi | |
| | pneumocytes | | 3-4+ (gr c) | 1+ (gr c > m) | |
| | 1+ (c) | | rare, | rare to occasional | |
| | very rare | | 2+ (gr c) | and ± (gr c > m) | |
| | | | occasional, | occasional to | |
| | | | and 1+ (gr c) | frequent | |
| | | | occasional | | |
| Cyno Liver #1 | hepatocytes | hepatocytes | hepatocytes | — | |
| | 1+ (c) | 2+ (gr c) | 2+ (gr c) | | |
| | frequent | rare to occasional | rare to occasional | | |
| | | and 1+ (gr c) | and 1+ (gr c) | | |
| | | rare to occasional | occasional; | | |
| | | | ductal epi | | |
| | | | 1+ (gr c) | | |
| | | | occasional | | |
| Cyno | epi and Islet Cells | — | islet cells ± | — | positive |
| Pancreas #1 | 1+ (c) | | (gr c) | | |
| | very rare | | frequent; | | |
| | | | ductal epi | | |
| | | | 1+ (gr c) | | |
| | | | rare to occasional | | |
| Cyno Heart #1 | myocardium | — | — | — | |
| | 1+ (c) | | | | |
| | frequent | | | | |
| Cyno Kidney | epi | tubule epi | tubule epi | — | positive |
| #070368M | 2+ (c) | 2+ (gr c) | 2+ (gr c) | | |
| | frequent | rare to occasional | rare to occasional | | |
| | | and 1+ (gr c) | and 1+ (gr c) | | |
| | | rare to occasional | occasional | | |
| | | | to frequent | | |
| Cyno Bladder | transitional | — | transitional cell epi | transitional cell epi | |
| #1 | epi ± (c); | | 2-3+ (gr c > m) | 2+ (gr c > m) | |
| | macrophages | | rare | rare | |
| | very rare | | and 1+ (gr c > m) | and 1+ (gr c > m) | |
| | | | occasional | rare to occasional | |

The results thus demonstrate that hMAB-A (2.2) exhibited an overall low-level staining of human hepatocytes and kidney tubules at optimal concentration, with a lower staining intensity/frequency of reactivity in hepatocytes and kidney tubules observed in the negative control. hMAB-A (2.2) exhibited similar low-level staining of cyno hepatocytes and kidney tubules at optimal concentration, with lower staining intensity/frequency of reactivity in kidney tubules observed in the negative control.

The results also demonstrate that hMAB-A (2C.2) exhibited an overall low-level staining of human hepatocytes and kidney tubules at optimal concentration, with lower staining intensity/frequency of reactivity in hepatocytes and kidney tubules observed in the negative control. hMAB-A (2C.2) exhibited similar low-level staining in cyno hepatocytes and kidney tubules at optimal concentration. Additional minimal findings in cyno lung epithelium, pancreas islets/epithelium and bladder epithelium for hMAB-A (2C.2) was not observed in the corresponding human tissue; lower staining intensity/frequency of reactivity was observed in lung epithelium, kidney tubules, bladder epithelium in negative control.

The results also demonstrate that hMAB-A (2I.2) exhibited no staining of human or cyno tissues at optimal concentration, with rare +/−bladder transitional cell epithelium staining. hMAB-A (2I.2) also exhibited overall low level and frequency staining of human lung alveolar cells, pancreas ductal epithelium, kidney tubule, bladder transitional cell epithelium at 5× optimal concentration, and overall low-level staining of cyno bronchial epithelium and bladder transitional cell epithelium at 5× optimal concentration. hMAB-A (2I.2) exhibited an overall favorable IHC profile on the human normal tissues tested and a similar profile on corresponding cynomolgus monkey tissues.

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. An ADAM9-binding molecule comprising a humanized ADAM9-binding domain, wherein said humanized ADAM9-binding domain comprises a Heavy Chain Variable (VH) Domain that comprises a CDR_{H}1 Domain, a CDR_{H}2 Domain and a CDR_{H}3 Domain, and a Light Chain Variable (VL) Domain that comprises a CDR_{L}1 Domain, a CDR_{L}2 Domain, and a CDR_{L}3 Domain, wherein:
(A) said CDR_{H}1 Domain, said CDR_{H}2 Domain and said CDR_{H}3 Domain comprise the amino acid sequences of SEQ ID NOs: 8, 35 and 37, respectively; and said CDR_{L}1 Domain, said CDR_{L}2 Domain and said CDR_{L}3 Domain comprise the amino acid sequences of SEQ ID NOs: 62, 13 and 14, respectively; or
(B) said CDR_{H}1 Domain, said CDR_{H}2 Domain and said CDR_{H}3 Domain comprise the amino acid sequences of SEQ ID NOs: 8, 35 and 38, respectively; and said CDR_{L}1 Domain, said CDR_{L}2 Domain and said CDR_{L}3 Domain comprise the amino acid sequences of SEQ ID NOs: 62, 13 and 14, respectively; or
(C) said CDR_{H}1 Domain, said CDR_{H}2 Domain and said CDR_{H}3 Domain comprise the amino acid sequences of SEQ ID NOs: 8, 35 and 39, respectively; and said CDR_{L}1 Domain, said CDR_{L}2 Domain and said CDR_{L}3 Domain comprise the amino acid sequences of SEQ ID NOs: 62, 13 and 14, respectively; or
(D) said CDR_{H}1 Domain, said CDR_{H}2 Domain and said CDR_{H}3 Domain comprise the amino acid sequences of SEQ ID NOs: 8, 35 and 40, respectively; and said CDR_{L}1 Domain, said CDR_{L}2 Domain and said CDR_{L}3 Domain comprise the amino acid sequences of SEQ ID NOs: 62, 13 and 14, respectively; or
(E) said CDR_{H}1 Domain, said CDR_{H}2 Domain and said CDR_{H}3 Domain comprise the amino acid sequences of SEQ ID NOs: 8, 35 and 41, respectively; and said CDR_{L}1 Domain, said CDR_{L}2 Domain and said CDR_{L}3 Domain comprise the amino acid sequences of SEQ ID NOs: 62, 13 and 14, respectively; or
(F) said CDR_{H}1 Domain, said CDR_{H}2 Domain and said CDR_{H}3 Domain comprise the amino acid sequences of SEQ ID NOs: 8, 35 and 42, respectively; and said CDR_{L}1 Domain, said CDR_{L}2 Domain and said CDR_{L}3 Domain comprise the amino acid sequences of SEQ ID NOs: 62, 13 and 14, respectively; or
(G) said CDR_{H}1 Domain, said CDR_{H}2 Domain and said CDR_{H}3 Domain comprise the amino acid sequences of SEQ ID NOs: 8, 35 and 43, respectively; and said CDR_{L}1 Domain, said CDR_{L}2 Domain and said CDR_{L}3 Domain comprise the amino acid sequences of SEQ ID NOs: 62, 13 and 14, respectively; or
(H) said CDR_{H}1 Domain, said CDR_{H}2 Domain and said CDR_{H}3 Domain comprise the amino acid sequences of SEQ ID NOs: 8, 35 and 44, respectively; and said CDR_{L}1 Domain, said CDR_{L}2 Domain and said CDR_{L}3 Domain comprise the amino acid sequences of SEQ ID NOs: 62, 13 and 14, respectively; or
(I) said CDR_{H}1 Domain, said CDR_{H}2 Domain and said CDR_{H}3 Domain comprise the amino acid sequences of SEQ ID NOs: 8, 35 and 46, respectively; and said CDR_{L}1 Domain, said CDR_{L}2 Domain and said CDR_{L}3 Domain comprise the amino acid sequences of SEQ ID NOs: 62, 13 and 14, respectively.

2. The ADAM9-binding molecule of claim 1, wherein said humanized ADAM9-binding domain comprises:
(A) said VH Domain that comprises the amino acid sequence of SEQ ID NO:20, SEQ ID NO: 21, SEQ ID NO:22, SEQ ID NO:23, SEQ ID NO:24, SEQ ID NO:25, SEQ ID NO:26, SEQ ID NO:27 or SEQ ID NO:29; and
(B) said VL Domain that comprises the amino acid sequence of SEQ ID NO:55.

3. The ADAM9-binding molecule of claim 1, wherein said molecule is a monospecific ADAM9-binding antibody or an ADAM9-binding fragment thereof.

4. The ADAM9-binding molecule of claim 1, wherein said molecule is a bispecific antibody.

5. The ADAM9-binding molecule of claim 1, wherein said molecule is a bispecific binding molecule comprising a covalently bonded complex of two, three, four or five polypeptide chains of an antibody.

6. The ADAM9-binding molecule of claim 1, wherein said molecule is a trivalent binding molecule comprising a covalently bonded complex of three, four, five, or six polypeptide chains of an antibody.

7. The ADAM9-binding molecule of claim 1, wherein said ADAM9-binding molecule comprises an Fc Region selected from the group consisting of:
IgG1 Fc Region, IgG2 Fc Region, IgG3 Fc Region, and IgG4 Fc Region.

8. The ADAM9-binding molecule of claim 7, wherein said Fc Region is a variant Fc Region that comprises, relative to a wild-type Fc Region, one or more amino acid modification(s) that:
(a) reduce(s) the affinity of the variant Fc Region for an FcγR, relative to that exhibited by the wild-type Fc Region; and/or
(b) enhance(s) the serum half-life of said ADAM9-binding molecule, relative to a comparable molecule comprising the wild-type Fc Region.

9. The ADAM9-binding molecule of claim 8, wherein said one or more amino acid modification(s) that reduce(s) the affinity of the variant Fc Region for an FcγR comprise:
(A) L234A;
(B) L235A; or
(C) L234A and L235A;
wherein said numbering is that of the EU index as in Kabat.

10. The ADAM9-binding molecule of claim 8, wherein said one or more amino acid modification(s) that enhance(s) the serum half-life of said ADAM9-binding molecule comprise:
(A) M252Y;
(B) M252Y and S254T;
(C) M252Y and T256E;
(D) M252Y, S254T and T256E; or
(E) K288D and H435K;
wherein said numbering is that of the EU index as in Kabat.

11. The ADAM9-binding molecule of claim 10, wherein said molecule comprises a variant IgG1 Fc Region that comprises amino acid modifications M252Y, S254T and T256E, wherein said numbering is that of the EU index as in Kabat.

12. The ADAM9-binding molecule of claim 1, wherein said molecule further comprises a human IgG Constant CL Kappa Domain having the amino acid sequence of SEQ ID NO:101.

13. The ADAM9-binding molecule of claim 1, wherein said molecule is bispecific and comprises:
(a) an epitope-binding site capable of immunospecific binding to an epitope of ADAM9, wherein said epitope-binding site capable of immunospecific binding to an epitope of ADAM9 comprises said humanized ADAM9-binding domain; and
(b) an epitope-binding site of an antibody capable of immunospecific binding to an epitope of a molecule present on the surface of an effector cell.

14. The ADAM9-binding molecule of claim 13, wherein said molecule comprises:
(a) two epitope-binding sites capable of immunospecific binding to epitope(s) of ADAM9, wherein each of said epitope-binding sites capable of immunospecific binding to an epitope of ADAM9 comprises said humanized ADAM9-binding domain; and
(b) two epitope-binding sites of antibody(s) capable of immunospecific binding to epitope(s) of a molecule present on the surface of an effector cell.

15. The ADAM9-binding molecule of claim 1, wherein said molecule is trispecific and comprises:
(a) one epitope-binding site capable of immunospecific binding to an epitope of ADAM9, wherein said epitope-binding site capable of immunospecific binding to an epitope of ADAM9 comprises said humanized ADAM9-binding domain;
(b) one epitope-binding site of an antibody capable of immunospecific binding to an epitope of a first molecule present on the surface of an effector cell; and
(c) one epitope-binding site of an antibody capable of immunospecific binding to an epitope of a second molecule present on the surface of an effector cell.

16. The ADAM9-binding molecule of claim 13, wherein said molecule present on the surface of an effector cell is CD2, CD3, CD8, TCR, or NKG2D.

17. A pharmaceutical composition that comprises an effective amount of the ADAM9-binding molecule of claim 1 and a pharmaceutically acceptable carrier, excipient or diluent.

18. A method for treating cancer associated with, or characterized by, the expression of ADAM9 in a subject comprising administering to said subject an effective amount of an ADAM9-binding molecule comprising a humanized ADAM9-binding domain, wherein said humanized ADAM9-binding domain comprises a Heavy Chain Variable (VH) Domain that comprises a CDR_{H}1 Domain, a CDR_{H}2 Domain and a CDR_{H}3 Domain, and a Light Chain Variable (VL) Domain that comprises a CDR_{L}1 Domain, a CDR_{L}2 Domain, and a CDR_{L}3 Domain, wherein:
(A) said CDR_{H}1 Domain, said CDR_{H}2 Domain and said CDR_{H}3 Domain comprise the amino acid sequences of SEQ ID NOs: 8, 35 and 37, respectively; and said CDR_{L}1 Domain, said CDR_{L}2 Domain and said CDR_{L}3 Domain comprise the amino acid sequences of SEQ ID NOs: 62, 13 and 14, respectively; or
(B) said CDR_{H}1 Domain, said CDR_{H}2 Domain and said CDR_{H}3 Domain comprise the amino acid sequences of SEQ ID NOs: 8, 35 and 38, respectively; and said CDR_{L}1 Domain, said CDR_{L}2 Domain and said CDR_{L}3 Domain comprise the amino acid sequences of SEQ ID NOs: 62, 13 and 14, respectively; or
(C) said CDR_{H}1 Domain, said CDR_{H}2 Domain and said CDR_{H}3 Domain comprise the amino acid sequences of SEQ ID NOs: 8, 35 and 39, respectively; and said CDR_{L}1 Domain, said CDR_{L}2 Domain and said CDR_{L}3 Domain comprise the amino acid sequences of SEQ ID NOs: 62, 13 and 14, respectively; or
(D) said CDR_{H}1 Domain, said CDR_{H}2 Domain and said CDR_{H}3 Domain comprise the amino acid sequences of SEQ ID NOs: 8, 35 and 40, respectively; and said CDR_{L}1 Domain, said CDR_{L}2 Domain and said CDR_{L}3 Domain comprise the amino acid sequences of SEQ ID NOs: 62, 13 and 14, respectively; or
(E) said CDR_{H}1 Domain, said CDR_{H}2 Domain and said CDR_{H}3 Domain comprise the amino acid sequences of SEQ ID NOs: 8, 35 and 41, respectively; and said CDR_{L}1 Domain, said CDR_{L}2 Domain and said CDR_{L}3 Domain comprise the amino acid sequences of SEQ ID NOs: 62, 13 and 14, respectively; or
(F) said CDR_{H}1 Domain, said CDR_{H}2 Domain and said CDR_{H}3 Domain comprise the amino acid sequences of SEQ ID NOs: 8, 35 and 42, respectively; and said CDR_{L}1 Domain, said CDR_{L}2 Domain and said CDR_{L}3 Domain comprise the amino acid sequences of SEQ ID NOs: 62, 13 and 14, respectively; or
(G) said CDR_{H}1 Domain, said CDR_{H}2 Domain and said CDR_{H}3 Domain comprise the amino acid sequences of SEQ ID NOs: 8, 35 and 43, respectively; and said CDR_{L}1 Domain, said CDR_{L}2 Domain and said CDR_{L}3 Domain comprise the amino acid sequences of SEQ ID NOs: 62, 13 and 14, respectively; or
(H) said CDR_{H}1 Domain, said CDR_{H}2 Domain and said CDR_{H}3 Domain comprise the amino acid sequences of SEQ ID NOs: 8, 35 and 44, respectively; and said CDR_{L}1 Domain, said CDR_{L}2 Domain and said CDR_{L}3 Domain comprise the amino acid sequences of SEQ ID NOs: 62, 13 and 14, respectively; or
(I) said CDR_{H}1 Domain, said CDR_{H}2 Domain and said CDR_{H}3 Domain comprise the amino acid sequences of SEQ ID NOs: 8, 35 and 46, respectively; and said CDR_{L}1 Domain, said CDR_{L}2 Domain and said CDR_{L}3 Domain comprise the amino acid sequences of SEQ ID NOs: 62, 13 and 14, respectively.

19. The method of claim 18, wherein said cancer is selected from the group consisting of: bladder cancer, breast cancer, cervical cancer, colorectal cancer, esophageal cancer, gastric cancer, head and neck cancer, liver cancer, non-small-cell lung cancer, myeloid cancer, ovarian cancer, pancreatic cancer, prostate cancer, renal cell carcinoma, thyroid cancer, testicular cancer, and uterine cancer.
